(19) United States
(12) Patent Application Publication
(10) Pub. No.: US 2022/0348934 A1
(43) Pub. Date: Nov. 3, 2022

(54) NON-RIBOSOMAL PEPTIDES AND SYNTHETASES AND METHODS OF PREPARATION AND USE THEREOF

(71) Applicant: VICTORIA LINK LIMITED, Wellington (NZ)

(72) Inventors: David ACKERLEY, Wellington (NZ); Mark CALCOTT, Wellington (NZ)

(73) Assignee: VICTORIA LINK LIMITED, Wellington (NZ)

(21) Appl. No.: 17/642,492

(22) PCT Filed: Sep. 11, 2020
(86) PCT No.: PCT/NZ2020/050102

§ 371 Date: Mar. 11, 2022

(30) Foreign Application Priority Data
Sep. 13, 2019 (AU) 2019903420

(51) Int. Cl. C12N 15/52; C12N 15/62; C12P 21/02

(52) U.S. Cl. CPC C12N 15/52; C12N 15/62; C12P 21/02; C12Y 603/02

(57) ABSTRACT

The present invention encompasses non-ribosomal peptides and non-ribosomal peptide synthetases, and the preparation and use thereof. Specifically encompassed are methods for preparing non-ribosomal peptide synthetases, methods for preparing non-ribosomal peptides from these enzymes, as well as polynucleotides used for producing these enzymes, libraries comprising these polynucleotides, and microbial strains comprising these polynucleotides.

### RELATED APPLICATION

This application claims priority to Australian patent application number 2019903420, filed 13 Sep. 2019, the entirety of which is hereby incorporated by reference herein.

### FIELD OF THE INVENTION

The present disclosure relates generally to non-ribosomal peptides and non-ribosomal peptide synthetases, and their preparation and use. More specifically, the present disclosure relates to non-naturally occurring synthetase enzymes for preparing non-ribosomal peptides, as well as means for preparing and using these enzymes.

### BACKGROUND OF THE INVENTION

Non-ribosomal peptide synthetases (NRPS) are enzymes found in many bacteria and fungi, and are known to catalyse the production of biologically active small peptides from amino acid or related monomer precursors without the need for a nucleic acid template (Finking and Marahiel 2004; Challis and Naismith 2004; Marahiel and Essen 2009). NRPS are very large proteins containing sets of modules, each of which consists of various functional domains such as adenylation (A), condensation (C), cyclization (Cy), thiolation (T), or thioesterase (TE) domains (Marahiel et al. 1997).

There has been significant interest in generating novel non-ribosomal peptides via recombination of the NRPS genes that encode their biosynthetic machinery. The first attempts to create artificial NRPS enzymes were substitutions of A-T domains into the second and seventh modules of the NRPS involved in the biosynthesis of the lipopeptide surfactin (Stachelhaus et al. 1995). Despite being able to detect modified non-ribosomal peptides using mass spectrometry, in each case the yield of modified lipopeptide was strongly reduced, to only trace levels (Schneider et al. 1998).

Subsequent research provided evidence that C domains have stringent specificity towards the substrate activated by their cognate A domains (Belshaw et al. 1999; Ehmann et al. 2000). For instance, the work by Belshaw et al provided evidence that the C domain of the second module of tyrocidine biosynthesis (which normally incorporates L-proline) is unable to tolerate leucine or phenylalanine as an acceptor substrate. This stringent specificity towards the acceptor substrate has been used to suggest C and A domains are inseparable, and hence that using fermentation for high-yield production of modified non-ribosomal peptides is not feasible using A domain substitution. This has been the accepted dogma in the field ever since Belshaw et al 1999 and Ehmann et al 2000 were published (e.g., Baltz, 2014; Calcott et al. 2014; Winn et al. 2016; Brown et al. 2018; Baltz 2018; Bozhüyük et al. 2018; Bozhüyük et al. 2019).

A further difficulty noted in making functional A domain substitutions has been based on the structure of the termination module of SrfA-C (Tanovic et al. 2008). The domains within NRPS enzymes are connected by peptide linkers that have low sequence identity. The linker regions between modules and between A and T domains have previously been used as recombination points for in vitro and in vivo engineering (Doekel and Marahiel, 2000; Mootz et al. 2000; Nguyen et al. 2006, Doekel et al. 2008). However, the structure of SrfA-C solved by Tanovic et al (2008) suggested the linker between C- and A domains is less tolerant for substitution. This structure identified that the linker region located between the C- and A domains forms is well-defined and L-shaped. Within the linker region was an 11-residue helix which was closely associated with the A domain surface. In addition to the well-defined linker region, the C and A domains were found to form a tight interface. The inflexible linker and tight interface were suggested as making it difficult to exchange individual A domains on a broad scale (Tanovic et al. 2008).

Based on the results related to acceptor site specificity, the structure of NRPS enzymes, and the results of engineering efforts to date, it has been suggested as a rule that C domain specificity or the C/A domain interface cannot be disturbed for functional non-ribosomal peptide production (Nguyen et al. 2006; Tanovic et al. 2008; Baltz, 2014; Calcott et al. 2014; Winn et al. 2016; Brown et al. 2018; Baltz 2018; Bozhüyük et al. 2018; Bozhüyük et al. 2019).

The inventors have previously found evidence that the C domain exerts a profound effect on substrate incorporation. This was based on recombination of PvdD, the bi-modular NRPS responsible for introduction of two L-Thr residues at the C-terminus of pyoverdine, the major siderophore of P. aeruginosa. When five different synonymous A domains were substituted into PvdD, all generated high levels of a wild type pyoverdine product, whereas nine different non-synonymous A domain substitutions all failed to yield any detectable modified peptide (Ackerley and Lamont, 2004; Calcott et al. 2014). Instead, the only product resulting from a majority of these non-synonymous substitutions was trace amounts of the wild type pyoverdine (i.e., still having two L-Thr residues at the C-terminus), with even conservative substitutions such as L-Ser not being accepted. At the time, this was interpreted as being due to low-level promiscuous activation of L-Thr by the non-synonymous A domains, with strict C domain proof-reading ensuring only this substrate could ultimately be incorporated into the growing peptide (Calcott et al. 2014). To bypass the presumed C domain proof-reading constraints, there were previous attempts at substitution of cognate C-A domain pairings into PvdD. No substitutions were successful within the first module, which was interpreted as being due to disruption of COM-domain sequences necessary for PvdD to associate with PvdJ, the enzyme immediately upstream in the pyoverdine NRPS assembly line (Calcott et al. 2014). Yet, when modifying the second module, it was possible to produce detectable yields of three pyoverdines from a total of ten (seven non-synonymous) recombinant NRPS constructs tested (Calcott et al. 2014; Calcott and Ackerley, 2015).

In recent experiments, researchers have attempted to bypass the constraints of C domain specificity using A-T-C domains as exchange units. A key condition for substituting these exchange units is that the substrate specificity of the C domain must be respected (Bozhüyük et al. 2018). This means each modified NRPS needs to be individually constructed and makes it difficult to modify enzymes when modules across multiple enzymes, which limits the usefulness of this approach. To bypass these limitations the authors developed a second method in which recombination points were located within the centre of the C domain (Bozhüyük et al. 2019). It was reasoned this would bypass the C domain specificity and allow substitutions to be made in a more generic manner. Using this method to generate a library predicted to produce 48 compounds resulted in the production of 7 of the predicted compounds. However, most of these were produced at low yield. Furthermore, only four modified strains out of fifty strains screened successfully produced modified compounds, i.e., a success rate of only 8%.

Despite C domain acceptor substrate specificity being a hypothesised barrier to A domain substitution, previous attempts to identify the substrate specificity of C domains using structural information and bioinformatics have been unsuccessful (Bloudoff et al. 2016; Bloudoff and Schmeing 2017; Süssmuth and Mainz 2017; Rausch et al. 2007). In particular, researchers have been unable to solve a crystal structure with the acceptor substrate or use structural information to identify the binding pocket (Brown et al. 2018). Moreover, the most successful NRPS domain substitutions have created only a small number of compounds at a time and therefore there remains a need to generate modified non-ribosomal peptides with a much higher rate of success and yield.

The present disclosure seeks to address this need or at least to provide the public with a useful alternative.

### SUMMARY OF THE INVENTION

As described herein, the present inventors have generated novel non-ribosomal peptides with an unprecedentedly high success rate, in two different NRPS systems. In addition, the inventors have constructed non-naturally occurring NRPS with unique and unexpected recombination strategies. These results are highly advantageous and also contradictory to the overriding dogma in the field.

In general aspects, the present disclosure encompasses non-naturally occurring non-ribosomal peptide synthetase (NRPS) polypeptides, as well as enzymes comprising these polypeptides, polynucleotides encoding these polypeptides, libraries comprising these polypeptides, methods for producing these polypeptides, and methods for producing peptides from these polypeptides.

In one particular aspect, the invention encompasses a non-naturally occurring non-ribosomal peptide synthetase (NRPS) module, which comprises, in an N-terminal to C-terminal direction: (1) an amino acid sequence from a first NRPS module comprising a C domain from the C1 motif to the C7 motif, joined to (2) an amino acid sequence from a second NRPS module comprising an A domain or a fragment thereof.

In other aspects:

The amino acid sequence from the second NRPS module begins at a site 1 to 24 amino acids, or 1 to 14 amino acids, following the terminal helix of the C domain of the first NRPS module.

The amino acid sequence from the second NRPS module comprises an A domain of the second NRPS module, the A domain encompassing the linker helix to the A10 motif.

The amino acid sequence from the second NRPS module begins at a site within the terminal helix of the C domain of the first NRPS module.

The amino acid sequence from the second NRPS module begins at a site within the linker helix of the A domain of the first NRPS module.

The amino acid sequence from the second NRPS module begins at a site between the terminal helix of the C domain of the first NRPS module and linker helix of the A domain of the first NRPS module, inclusive.

The amino acid sequence from the second NRPS module begins at a site immediately following the C-terminus of the terminal helix of the C domain of the first NRPS module.

The amino acid sequence from the second NRPS module begins at a site immediately preceding the N-terminus of the linker helix of the A domain of the first NRPS module.

The amino acid sequence from the second NRPS module begins at a site immediately preceding the C-terminus of the linker helix of the A domain of the first NRPS module.

The amino acid sequence from the second NRPS module ends at a site preceding the first helix of the T domain of the first NRPS module.

The amino acid sequence from the second NRPS module ends at a site in the first NRPS module encompassing: the residue immediately following the A domain binding pocket to 20 residues following the A10 motif.

The amino acid sequence from the second NRPS ends at a site in the first NRPS module encompassing: the residue immediately following the A domain binding pocket to 10 residues following the A10 motif.

The first NRPS module and the second NRPS module have different substrate specificity.

The A domain of the first NRPS module and the A domain of the second NRPS module share less than 40%, less than 50%, less than 60% or less than 70% amino acid sequence identity.

The C domain of the first NRPS module and the C domain of the second NRPS module share less than 40%, less than 50%, less than 60% or less than 70% amino acid sequence identity.

The region intervening the A domain and the C domain of the first NRPS module and the region intervening the A domain and the C domain of the second NRPS module share less than 40%, less than 50%, less than 60% or less than 70% amino acid sequence identity.

The A domain binding pocket of the second NRPS module differs from the A domain binding pocket of the first NRPS module by 1 or more amino acids.

The A domain binding pocket of the second NRPS module differs from the A domain binding pocket of the first module by 2 or more, 3 or more, 4 or more, 5 or more, 6 or more, 7 or more, or 8 amino acids.

The one or more different amino acids in the A domain of the second NRPS module are one or more of the eight amino acids that determine the specificity of the A domain.

The downstream to amino acid sequence from the second NRPS module includes a C-terminal sequence from the first NRPS module.

The C-terminal sequence comprises a domain from the first NRPS module.

The downstream to amino acid sequence from the second NRPS module includes a C-terminal sequence from a third NRPS module.

The C-terminal sequence comprises a domain from the third NRPS module.

In yet other aspects:

The non-naturally occurring non-ribosomal peptide synthetase (NRPS) module has enzymatic activity.

As particular aspects:

The invention encompasses an enzyme comprising the non-naturally occurring NRPS module of any one of the preceding aspects.

The invention encompasses a polynucleotide comprising a nucleic acid sequence encoding the non-naturally occurring NRPS module of any one of the preceding aspects.

The invention encompasses a nucleic acid construct comprising a nucleic acid sequence encoding the non-naturally occurring NRPS module of any one of the preceding aspects.

The invention encompasses a library of nucleic acid constructs, wherein a nucleic acid construct in the library encodes a non-naturally occurring NRPS module of any one of the preceding aspects.

The invention encompasses a host cell comprising a nucleic acid construct of any one of the preceding aspects.

The invention encompasses a method for generating the non-naturally occurring NRPS module of any one of the preceding aspects.

The invention encompasses a method for production of a non-ribosomal peptide, the method comprising culturing the host cell according to a preceding aspect to produce the non-ribosomal peptide.

The invention encompasses a method for the production of a non-ribosomal peptide, the method comprising the use of the non-naturally occurring NRPS module of any one of the preceding aspects, the nucleic acid construct according to a preceding aspect, the library according to a preceding aspect, or the host cell according a preceding aspect.

The invention encompasses a kit comprising the non-naturally occurring NRPS module of any one of the preceding aspects, the nucleic acid construct according to a preceding aspect, the library according to a preceding aspect, or the host cell according a preceding aspect Novel features that are believed to be characteristic of the invention will be better understood from the detailed description of the invention when considered in connection with any accompanying figures and examples. However, the figures and examples provided herein are intended to help illustrate the invention or assist with developing an understanding of the invention; these are not intended to limit the invention's scope.

### BRIEF DESCRIPTION OF THE DRAWINGS

FIG. 1: Alignment of the amino acid sequences of the second module of PvdD (SEQ ID NO: 1) and the first module of TycB (SEQ ID NO: 2). The regions encoding the C, A and T domains are annotated along with the conserved motifs according to Marahiel et al (1997) and position of the terminal helix of the C domain. The region corresponding to the linker helix found to be associated with the A domain by Tanovic et al (2008) is also highlighted.

FIGS. 2A-B: A) Amino acid sequence alignment of the C domain from the second module of PvdD (regions 1-3; SEQ ID NO:122-124), and the C domain from the first module of PvdJ (regions 1-3; SEQ ID NO:125-127). The alignment is separated into three regions based on the highlighted low homology stretches. B) Homology model of the C domain from the second module of PvdD. The conserved histidine residue and residues differing between the Lys and Thr C domains from panel A are shown as sticks.

FIGS. 3A-B: A) Semi-rational shuffling approach used to narrow down substrate specificity regions. The three variable regions of the C domains from the Lys-specific module (solid boxes) and the Thr-specific module (empty boxes) were shuffled in every possible combination to create eight C domains. These were inserted into the plasmid pDEC-Lys. B) Pyoverdine production from strains containing shuffled C domains as assessed by measuring absorbance at 400 nm relative to a wild-type P. aeruginosa strain. Error bars represent the standard deviation from six independent replicates.

FIGS. 4A-B: A) Homology models showing the conserved catalytic histidine residue and residues modified within the third region of the C domain as spheres. B) Levels of pyoverdine production from strains containing modifications to the third variable region of the C domain. Production was assessed by measuring absorbance at 400 nm relative to a wild-type P. aeruginosa strain. Error bars represent the standard deviation from six independent replicates.

FIG. 5: Pyoverdine yield of strains containing C-A domain substitutions versus strains containing linker plus A domain substitutions. Production was assessed by measuring absorbance at 400 nm relative to a wild-type P. aeruginosa strain. Error bars represent the standard deviation from six independent replicates.

FIGS. 6A-B: A) Levels of pyoverdine production resulting from nine A domain substitutions into the second module of PvdD, as measured by absorbance at 400 nm. Error bars represent the standard deviation from six independent replicates. B) Mass spectra showing the production of modified pyoverdines.

FIG. 7: NRPS modules used as a source of sequences for phylogenetic analysis. Modules are from NRPS pathways involved in the biosynthesis of pyoverdine from i) P. aeruginosa PAO1, ii) P. syringae pv. phaseolicola 1448A, iii) P. putida KT2440 and iv) P. fluorescens SBW25. The substrate specificity and name of each module are located below the NRPS schematic. Modules within the same pathway exhibiting the same substrate specificity are labelled A to J.

FIGS. 8A-C: A) Maximum likelihood phylogenetic tree of the C domains from the modules shown in panel A. Domains are labelled according to the names in panel A and shaded according to the substrate specificity of the corresponding A domain. Letters A to J indicate modules having the same substrate specificity within the same pathway. B) Maximum likelihood phylogenetic tree of the A domains from the modules shown in panel A. Shading and labelling is identical to panel B. C) Key showing the shading used to indicate substrate specificity in panels B and C.

FIGS. 9A-B: A) Phylogenetic compatibility matrices of alignments of C-A-T domains from Pseudomonas, Bacillus and Streptomyces species showing frequencies of phylogeny violations for each pairwise comparison of sequence fragments. A bootstrap value of 70% was used to calculate phylogenetic violations. B) Segregation of alignments by consensus substrate specificity. The segregation score was calculated using a 0%, 50% and 70% bootstrap cut off. The locations of key conserved motifs are indicated along the top of the graph. Shaded blocks have been added to aid comparison between regions of interest.

FIG. 10: Recombination hotspot analysis of C-A-T domains from Pseudomonas, Bacillus and Streptomyces species. ‘X’ marks the location of recombination found to allow the successful Lys A domain substitution. Dark and light grey areas indicate local breakpoint hotspots at the 95% and 99% confidence level, and the two horizontal lines indicate cut-offs for global breakpoint hotspots at the 95% and 99% confidence level.

FIGS. 11A-B: A) Pyoverdine production of partial A domain substitutions as measured by absorbance at 400 nm. Error bars represent the standard deviation from three independent replicates. B) The average number of clashes calculated using SCHEMA which were introduced by recombination of 9 modules with the domains from the second module of PvdD. The dark shaded region of the graph indicates 1 standard deviation. Lines labelled 1 to 6 show the approximate locations of substitutions tested in Panel A.

FIGS. 12A-C: A) Diagram representing the upstream recombination points tested for A domain substitution. ‘X’ refers to the site originally identified, and ‘A’ to ‘D’ represent the additional sites tested. B) Pyoverdine production of previous C-A domain substitutions (Calcott and Ackerley, 2015) and two additional strains expressing pvdD constructs bearing Gly or Phe CA domain substitutions, in comparison with the A domain substitutions using the upstream sites identified in Panel A. Levels of pyoverdine were measured by optical density at 400 nm. Error bars represent the standard deviation from three independent replicates. C) Diagram highlighting the recombination points used showing the recombination points tested using PvdD in panel B. ‘X’ and ‘A’ to ‘D’ sites are described for Panel A, above. The equivalent sites are identified in module 1 of TycB by sequence alignment. The dashed black rectangle indicates the preferred recombination region bounded by the N-terminus of the terminal helix of the C domain and the C-terminus of the linker helix of the A domain. The smaller solid black rectangle indicates the more preferred recombination sites bounded by the C-terminus of the terminal helix and the N-terminus of the linker helix.

FIGS. 13A-D: A) A diagram representing the NRPS enzymes used to make the cyclic Phe-Pro dipeptide. B) HPLC analysis showing production of the cyclic Phe-Pro dipeptide by the inventors labelled 1, in comparison to a cyclic Phe-Pro standard. Quantification was performed based on 3 independent replicates. C) A diagram representing the NRPS enzymes used to make a linear Phe-Leu dipeptide. D) HPLC analysis showing production of the Phe-Leu dipeptides labelled 2-6, in comparison to a Phe-Leu standard. Quantification was performed based on 3 independent replicates.

FIG. 14: Relative levels of pyoverdine production from strains expressing pvdD constructs bearing four additional A domain substitutions at the X, B or D sites identified in FIG. 12A. Levels of pyoverdine were measured by optical density at 400 nm. Error bars represent the standard deviation from three independent replicates.

FIG. 15: HPLC analysis showing production of D-Phe-L-Leu dipeptides resulting from A-domain substitutions at the X, B or D sites in the PheAT-ProCATTe dimodular system, using the three Leu-specifying A domains labelled 2, 3 and 5 for TycC6, SrfAC and NZ_CP020028.1.cluster004, respectively.

FIGS. 16A-B: A) Diagram highlighting the downstream recombination points, D1 to D7 and A10 located downstream of the conserved A10 motif. Sites are identified in module 2 of PvdD and module 1 of TycB by sequence alignment. B) Pyoverdine production of A domain substitutions using downstream sites D1 to D7 identified in panel A, in comparison to the previously used A10 site. Each substitution was generated in combination with the upstream B site. Levels of pyoverdine production were measured by optical density at 400 nm. Asterisks indicate strains in which the modified pyoverdine predicted by the substituted A domain was detected by MALDI mass spectrometry. Error bars represent the standard deviation from three independent replicates.

### DETAILED DESCRIPTION OF THE INVENTION

The following description sets forth numerous exemplary configurations, parameters, and the like. It should be recognised, however, that such description is not intended as a limitation on the scope of the present invention; it is instead provided as a description of exemplary embodiments.

### Definitions

Throughout the specification and claims, the following terms take the meanings explicitly associated herein, unless the context clearly dictates otherwise.

The various examples, embodiments, and aspects as set out herein may be readily combined, without departing from the scope or spirit of the invention. Thus, the phrase “in one example”, “in one embodiment”, or “in one aspect” is not necessarily exclusive of other examples, embodiments, or aspects that are also described. In the same way, the phrase “in another example”, “in another embodiment”, or “in another aspect” is not necessarily exclusive of other examples, embodiments, or aspects that are described.

In each instance herein, in descriptions, embodiments, and examples of the present invention, the terms “comprising”, “including”, etc, are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

Where a range is given in the specification, for example, a temperature range, a time range, or a composition range, all intermediate ranges and subranges, as well as all individual values included in the ranges given are intended to be included in the disclosure. Thus, each range that is specified (e.g., 1 to 10) includes all possible combinations of numerical values between the lowest value and the highest value enumerated (e.g., 1, 1.1, 2, 3, 3.3, 4, 5.5, 6, 7, 8.9, 9 and 10) and also any range of rational numbers within that range (e.g., 2 to 8, 1.5 to 5.5, and 3.1 to 4.9), and, therefore, all sub-ranges of all ranges expressly disclosed herein are hereby expressly disclosed. The numeric values provided in parentheses here are only examples of what is specifically intended and all possible combinations of numerical value between the lowest value and the highest value enumerated are to be considered to be expressly stated in this disclosure in a similar manner.

As used herein “and/or” means additionally or alternatively.

As used herein “based on” is not exclusive and allows for being based on additional factors not described, unless the context clearly dictates otherwise. The meaning of “in” includes “in” and “on.”

Any use of a term in the singular also encompasses plural forms. Thus, throughout the specification, the meaning of “a”, “an”, and “the” include plural references.

The term “about” or “approximately” means up to 10% greater than or up to 10% lesser than a particular value.

As used herein, an “isolated” component (e.g., isolated peptide or isolated enzyme) refers to a component that has been purified from (e.g., separated from) other components. An isolated component may have: about 70% purity or greater, about 80% purity or greater, about 90% purity or greater; or, in particular aspects, about 99% purity or greater. An isolated component may be obtained by any method or combination of methods as known and used in the art, including biochemical, recombinant, and synthetic techniques.

“Isolated” as used herein with reference to polynucleotide, peptide, or polypeptide sequences describes a sequence that has been removed from its originating environment, e.g., natural cellular environment or synthetic environment. The polynucleotide, peptide, or polypeptide sequences of this disclosure may be prepared by at least one purification step.

“Isolated” when used herein in reference to a cell or host cell describes to a cell or host cell that has been obtained or removed from an organism or from its natural environment and is subsequently maintained in a laboratory environment as known in the art. The term encompasses single cells, per se, as well as cells or host cells comprised in a cell culture and can include a single cell or single host cell.

The term “construct”, e.g., “genetic construct”, refers to a polynucleotide molecule, usually double-stranded DNA, which may have cloned or inserted into it another polynucleotide molecule. For example, a construct may have an unidentified polynucleotide insert that is prepared from an environmental sample or as a cDNA, but not limited thereto. A construct may contain the necessary elements that permit transcription of a cloned or inserted polynucleotide molecule, and, optionally, for translating the transcript into a peptide or polypeptide. The inserted polynucleotide molecule may be derived from the host cell, or may be derived from a different cell or organism. Once inside the host cell the construct may become integrated in the host chromosomal DNA. The construct may be linked to a vector.

The term “vector” as used herein refers to a polynucleotide molecule, usually double stranded DNA, which is used to replicate or express a construct. The vector may be used to transport a construct into a given host cell.

The term “polynucleotide(s),” as used herein, means a single or double-stranded deoxyribonucleotide or ribonucleotide polymer of any length, and include as non-limiting examples, coding and non-coding sequences of a gene, genomic DNA, recombinant polynucleotides, isolated and purified naturally occurring DNA or RNA sequences, synthetic RNA and DNA sequences, fragments, constructs, and vectors. Reference to nucleic acids, nucleic acid molecules, nucleotide sequences, and polynucleotide sequences is to be similarly understood.

The term “polypeptide”, as used herein, encompasses amino acid chains of any length, wherein the amino acid residues are linked by covalent peptide bonds. “Polypeptide” may refer to a polypeptide that is a purified natural product, or that has been produced partially or wholly using recombinant or synthetic techniques. The term may refer to an aggregate of a polypeptide such as a dimer or other multimer, a fusion polypeptide, a polypeptide fragment, a polypeptide variant, modification, fragment, or derivative thereof. The term “polypeptide” is used interchangeably herein with the terms “peptide” and “protein”.

A “fragment” of a polypeptide is a subsequence of a particular polypeptide. In certain aspects, the fragment is a functional fragment. A functional fragment performs a function that is required for a biological activity or binding and/or provides three dimensional structure of the polypeptide. The term may refer to a polypeptide fragment, an aggregate of a polypeptide fragment, a fusion polypeptide fragment, a fragment of a polypeptide variant or modification, or a fragment of a polypeptide derivative thereof that is capable of performing the polypeptide activity.

The term “full length” as used herein with reference to a sequence means a peptide or polypeptide that comprises a contiguous sequence of amino acid residues where each amino acid residue has been expressed from each of its corresponding codons in the polynucleotide over the entire length of the coding region and resulting in a fully functional polypeptide, peptide, or protein. As will be appreciated by a person of ordinary skill in the art, a “full length” sequence contains the amino acid sequence that corresponds to and has been expressed from each and every codon encoded by the polynucleotide comprising the entire coding region of the polypeptide, wherein each of said codons is located between the start codon and the termination codon normally associated with that coding region.

The term “expressing” refers to the expression of a nucleic acid transcript from a nucleic acid template and/or the translation of that transcript into a peptide or polypeptide, and is used herein as commonly used in the art.

The term “incubating” refers to the placing together of elements so they may interact and is used herein as commonly used in the art.

The term “endogenous” as used herein refers to a constituent of a cell, tissue or organism that originates or is produced naturally within that cell, tissue or organism. An “endogenous” constituent may be any constituent including but not limited to a polynucleotide, a polypeptide, or a peptide, including a non-ribosomal peptide, but not limited thereto.

The term “exogenous” as used herein refers to any constituent of a cell, tissue or organism that does not originate or is not produced naturally within that cell, tissue or organism. An exogenous constituent may be, for example, a polynucleotide sequence that has been introduced into a cell, tissue or organism, or a peptide or polypeptide expressed in that cell, tissue or organism from that polynucleotide sequence.

“Naturally occurring” as used herein with reference to a polynucleotide or polypeptide sequence according to the invention refers to a sequence that is found in nature. A synthetic sequence that is identical to a wild-type sequence is, for the purposes of this disclosure, considered a naturally occurring sequence. A naturally occurring sequence also refers to a variant sequence as found in nature that differs from wild-type. For example, allelic variants and naturally occurring sequences due to hybridization or horizontal gene transfer, and variants arising out of other natural processes. What is important for a naturally occurring sequence is that the actual sequence (e.g., nucleotide or amino acid sequence) is found or known from nature.

“Non-naturally occurring” as used herein with reference to a polynucleotide or polypeptide sequence according to the invention refers to a sequence that is not found in nature. Examples of non-naturally occurring sequences include artificially produced and variant sequences, made for example by recombination, domain swapping, point mutation, insertion, deletion, or other methods, or combinations of these methods. Non-naturally occurring sequences also include chemically evolved sequences. What is important for a non-naturally occurring sequence according to the invention is that the actual sequence (e.g., nucleotide or amino acid sequence) is not found or known from nature.

The term, “wild-type” when used herein with reference to a polynucleotide refers to a naturally occurring, non-mutant form of a polynucleotide, peptide, polypeptide, or organism. A wild-type peptide or polypeptide is capable of being expressed from a wild-type polynucleotide. In one embodiment, a wild-type polypeptide is a wild-type NRPS polypeptide that is expressed from a wild-type polynucleotide.

“Homologous” as used herein with reference to polynucleotide regulatory elements, means a polynucleotide regulatory element that is a native and naturally-occurring polynucleotide regulatory element. A homologous polynucleotide regulatory element may be operably linked to a polynucleotide of interest such that the polynucleotide of interest can be expressed from a, vector, construct, or expression cassette according to the invention.

“Heterologous” as used herein with reference to polynucleotide regulatory elements, means a polynucleotide regulatory element that is not a native and naturally-occurring polynucleotide regulatory element. A heterologous polynucleotide regulatory element is not normally associated with the coding sequence to which it is operably linked. A heterologous regulatory element may be operably linked to a polynucleotide of interest such that the polynucleotide of interest can be expressed from a vector, construct, or expression cassette according to the invention. Such promoters may include promoters normally associated with other genes, ORFs or coding regions, and/or promoters isolated from any other bacterial, viral, eukaryotic, or mammalian cell.

The term “recombinant” refers to a polynucleotide sequence that is removed from sequences that surround it in its natural context and/or is recombined with sequences that are not present in its natural context. A “recombinant” peptide or polypeptide sequence is produced by translation from a “recombinant” polynucleotide sequence.

As used herein, the term “variant” refers to polynucleotide, peptide, or polypeptide sequences different from the specifically identified sequences, wherein one or more nucleotides or amino acid residues is deleted, transposed, substituted, or added. Variants may be naturally occurring allelic variants, or non-naturally occurring variants. Variants may be from the same or from other species and may encompass homologues, paralogues, and orthologues. In certain embodiments, the variants useful in the invention have biological activities that are the same or similar to those of a corresponding wild-type molecule; i.e., functional variants of the parent peptide, polypeptide, or polynucleotide. In certain embodiments, the variants have biological activities that differ from their corresponding wild-type molecules. In certain embodiments, the differences are altered activity and/or binding specificity. For example, a functional NRPS polypeptide variant may produce a particular peptide. In certain embodiments, the levels of NRP produced by the functional variant may be higher or lower than produced by the wild-type NRPS polypeptide.

The term “variant” with reference to polynucleotides, peptides, and polypeptides encompasses all forms of polynucleotides, peptides, and polypeptides as defined herein.

As used herein, the term “mutagenesis” refers to methods to alter a polynucleotide sequence either in vitro or in vivo, most commonly to change the sequence of one or more polypeptides encoded therein. Mutagenesis methods include as non-limiting examples, error-prone PCR, DNA shuffling, chemical mutagenesis, application of ultraviolet radiation, genome shuffling, and use of mutator strains. In one application, mutagenesis may be followed by high-throughput screening to enable recovery of improved variants, for example strains of bacteria that as a consequence of mutagenesis now exhibit increased levels of production of glutamine or an analogue thereof.

As used herein, the term in vitro refers to a reaction performed outside of the confines of a living cell or a host organism.

As used herein, the term in vivo refers to a reaction performed within a living cell and/or within a host organism.

The term “high throughput screening” as used herein refers to a significant increase in number of results that can be generated by a given method, in comparison to other methods used to generate the same, or same type of results. For example, methods may be used to screen about 50, about 75, about 100, about 250, about 500, about 1000, or about 10,000 to about 100,000 candidates per day, or at least 50 candidates per day, at least 50 candidates per day, at least 75 candidates per day, at least 100 candidates per day, at least 250 candidates per day, at least 500 candidates per day, at least 1000 candidates per day, at least 10,000 candidates per day, at least 100,000 candidates per day, but not limited thereto.

“Activation” refers to any action or change that causes a substrate to adopt a functional conformation or perform a functional role that the substrate was not capable of performing before being activated. For example, an NRPS polypeptide as described herein may be considered a substrate that is activated by a PPTase. An NRPS is considered “activated” for the purposes of the invention, when it has had a 4′-phosphopantetheine (4′-PP) cofactor attached by a PPTase. Activation of an NRPS polypeptide means the same thing.

The term “non-ribosomal peptide synthetase” (NRPS) refers to a biosynthetic enzyme that catalyses the addition of a constituent to a non-ribosomal peptide, for example an amino acid constituent. NRPS are exemplified by synthetase enzymes, for example, bacterial synthetase enzymes, including Bacillus (e.g., Brevibacillus), Pseudomonas, and Streptomyces synthetase enzymes. Also noted are Burkholderia, Xenorhabdus, and Photorhabdus enzymes. Soil dwelling bacterial strains and their NRPS are particularly noted. Exemplifications include but are not limited to: enzymes that synthesise protease or proteasome inhibitors, for example, 20S proteasome inhibitors, as well as enzymes that synthesise siderophores, and enzymes that synthesise antibiotic peptides, and any modifications of these, as described in detail herein.

The term “modified NRPS” refers to an NRPS that is not a naturally occurring variant of a wild-type NRPS. In the same way, a “modified NRPS polypeptide” refers to a NRPS polypeptide that is not a naturally occurring variant of a wild-type NRPS polypeptide. Modification may be carried out in accordance with the disclosed methods. For example, various methods of recombination may be used to achieve modification. Modified NRPS and NRPS polypeptides useful in the invention may have biological activities that are the same or similar to those of a corresponding wild-type molecule i.e., functional modifications. Alternatively, modified NRPS and NRPS polypeptides may have biological activities that differ from their corresponding wild-type molecules. In certain embodiments, the differences are altered activity and/or binding specificity. For example, a functional modification may produce a particular NRP. In certain embodiments, the levels of NRP produced by the functional modification may be higher or lower than produced by the wild-type molecule. In particular embodiments, a modified NRPS may comprise a recombinant NRPS, a modified NRPS polypeptide may comprise a recombinant NRPS polypeptide, and a modified NRPS module may comprise a recombinant NRPS module, as set out in this description.

The term “non-ribosomal peptide” (NRP) refers to biologically active small peptides or molecules derived from biologically active small peptides that are synthesised by non-ribosomal peptide synthetases (NRPS) from amino acid precursors wherein the non-ribosomal peptide itself is not directly encoded by a polynucleotide template. NRPs are exemplified by siderophores such as pyoverdines; antibiotics such as tyrocidines and gramicidins, e.g., Gramicidin S; protease or proteasome inhibitors such as eponemycin, epoxomicin, and syrbactins, e.g., syringolin and glidobactin, and any variants of any of the above, as described in detail herein.

The terms “A domain”, “C domain”, “T domain”, and “TE domain” as used herein refer to peptide domains that can be defined as regions of amino acid sequence within NRPS enzymes that contain a majority of the motif sequences for each domain type as defined by Marahiel et al. 1997. Reviewed in Süssmuth and Mainz 2017. The term “T domain” refers to the NRPS domain that is the site of attachment of the 4′-PP cofactor, as above. The term T domain is used interchangeably with peptidyl carrier protein domain (PCP domain) and carrier protein domain (CP domain). Multiple domains can be combined to make an NRPS “module”.

A “modified A domain” refers to an A domain with one or more sequence modification as described herein. For example a modified domain may have one or more linker sequence upstream from the domain sequence, one or more linker sequence downstream from the domain sequence. In certain embodiments, an A domain or fragment thereof from a first NRPS module is substituted with the A domain or fragment thereof from a second NRPS module. Accordingly, A domain substitutions are included as modified A domains. Exemplifications of modified NRPS domains are set out in detail herein.

The term “A domain binding pocket” refers to the configuration of amino acids within the active site of an A domain of an NRPS polypeptide. The binding pocket defines the substrate specificity of the A domain (Stachelhaus et al, 1999; Challis et al, 2000). Substrate specificity will have been established, or can be determined experimentally. See, e.g., Khayatt et al. 2013. The term “A domain coding residues” refers to the specific identities of the eight amino acids that determine the specificity of a particular A domain. These were identified by Stachelhaus et al (1999) as residues Ala236, Trp239, Thr278, Ile299, Ala301, Ala322, Ile330 and Cys331 of PheA. The corresponding residues can be determined by sequence alignment to PheA or by using software packages including but not limited to 2MetDB.

It is understood that, for any DNA molecule disclosed herein, the corresponding RNA molecule and peptide/polypeptide molecules are also encompassed and disclosed. Likewise, for any peptide/polypeptide molecule disclosed herein, the corresponding RNA and DNA sequences are also considered to be encompassed and disclosed. In addition, where there are multiple sequence identifiers, e.g., “SEQ ID NO: 122-127”, this format may be understood as referring to each sequence individually, or any combination thereof.

### NRP and NRPS Polypeptides

Non-ribosomal peptides (NRPs) are a class of small peptide natural products synthesised mainly by bacteria and fungi. Despite their small size, they are highly diverse in terms of the monomers that can be incorporated. As of 2014 there were 1164 different non-ribosomal peptides known (Caradec et al. 2014), which collectively contain over 500 unique monomers, including both proteinogenic and non-proteinogenic L- and D-amino acids, as well as carboxylic acids and amines (Caboche et al. 2010). Non-ribosomal peptides also exhibit high structural diversity with only 27% being linear; the remainder having cyclic, branched or other complex primary structures (Caboche et al. 2010).

The diversity of non-ribosomal peptides imparts on them many properties of relevance to biotechnology; for example, peptides have been identified with antibiotic, antiviral, anti-cancer, anti-inflammatory, immunosuppressant and surfactant qualities (Sieber and Marahiel 2005; Felnagle et al. 2008). Importantly for medicine, natural products often need to be modified to improve clinical properties and/or bypass resistance mechanisms (Bush 2012; O'Connell et al. 2013). Due to their typically complex structures, most clinical natural product derivatives are created by means of semisynthesis; a process whereby the natural product is chemically modified post-isolation from biological sources (Kirschning and Hahn 2012; O'Connell et al. 2013). An alternative synthetic strategy, which would also open up a wide range of structural diversity, is the use of protein engineering to modify the genetic templates that specify these natural products. Non-ribosomal peptides have a modular mode of synthesis, which makes them potentially amenable to rational manipulation at the genetic level. However, to date most attempts to achieve this have yielded a biosynthetic machinery that is either greatly impaired in its activity, or completely non-functional.

Non-ribosomal peptide synthesis generally follows the multiple template model, originally proposed by Stein et al. (1994). According to this model, peptides are synthesised in a modular assembly line-like manner by NRPS enzymes (“the template”). The modules that comprise an NRPS template may be clustered on a single enzyme or located within multiple distinct enzymes that associate post-translation; and are classified as either initiation, termination, or elongation modules depending on their location in the assembly line. Modules act in a concerted but semi-autonomous fashion, and are defined by their ability to recognise, activate and incorporate a specific monomer into the final peptide product (Hur et al. 2012).

Within each module of an NRPS, an adenylation (A) domain recognises and activates a specific substrate by addition of AMP. The activated substrate is then tethered to a flexible 4′-phosphopantetheine (PPT) prosthetic group, which is itself covalently attached to a thiolation (T) domain (also known as a peptidyl carrier protein (PCP) domain). The T domain lies at the heart of the biosynthetic process, with its flexible PPT prosthesis effectively the “swinging arm” of a biomolecular assembly line that transfers peptide intermediates between different domains and modules. Post-attachment of an activated substrate by its A domain partner, a T domain then passes that substrate to a condensation (C) domain, which catalyses peptide bond formation between the donor substrate provided by the T domain immediately upstream, and the acceptor substrate provided by the downstream T domain.

Following the initial condensation event, the process can repeat in an iterative fashion, with the previous peptide intermediate now serving as the donor substrate for the C domain of the next module in an NRPS complex. Along the way, certain modules may contain additional tailoring domains that modify individual substrates in a directed fashion (e.g., epimerisation (E) domains, for conversion from L- to D-enantiomers). The growing peptide continues to be passed from the T domain of one module to the T domain of the next until the product is released, typically via a hydrolysis or intramolecular cyclisation reaction catalysed by a thioesterase (TE) domain associated with the final module in an NRPS complex.

The gram-negative bacteria Pseudomonas aeruginosa produces two major siderophores. One is pyochelin (Pch), which is a derivative of salicylic acid (Cox et al. 1981), and the other is pyoverdine (Pvd) (Ankenbauer et al. 1985). P. aeruginosa strains produce several pyoverdine peptides, which can be classified into three types (PvdI to PvdIII) and can be distinguished by their amino acid sequences (Meyer et al. 1997). For pyoverdine sequences, the peptide and the chromophore are derived from amino acid precursors that are assembled by non-ribosomal peptide synthetases (NRPSs), with other enzymes catalysing additional reactions to complete the maturation of the pyoverdine peptides (Ackerley et al. 2003; Beare et al. 2003; Cunliffe et al. 1995; Handfield et al. 2000; McMorran et al. 2001; McMorran et al. 1996; Miyazaki et al. 1995; Visca et al. 1994).

Pyoverdines comprise an (1S)-5-amino-2,3-dihydro-8,9-dihydroxy-1H-pyrimido[1,2-a]quinoline core, and can include a 6- to 12-amino acids chain (Meyer, 2000; Ravel and Cornelis, 2003). In Pseudomonas aeruginosa strain PAO1, pyoverdine peptide synthesis involves four NRPS (Georges and Meyer 1995), PvdL, PvdI, PvdJ and PvdD, which direct the synthesis of a pyoverdine precursor peptide of 11 amino acids with the sequence L-Glu-L-Tyr-D-Dab-L-Ser-L-Arg-L-Ser-L-fOHOrn-L-Lys-L-fOHOrn-L-Thr-L-Thr and with the second and third amino acids of the peptide (L-Tyr and D-Dab-D-amino butyric acid-) forming the chromophore (see, e.g., Georges and Meyer 1995; Lehoux et al. 2000; Demitris et al. 2002; Ackerley et al. 2003; Lamont et al. 2003).

The soil bacterium Brevibacillus brevis produces an antibiotic composition known as tyrocidine. This is made as part of a mixture called tyrothricin, consisting of tyrocidine and the linear pentadecapeptide gramicidin. Tyrocidine itself is a mixture of at least four known structural variants. Tyrocidine A, the most prominent of these, is a cyclic decapeptide with the primary structure (-DPhe-Pro-Phe-DPhe-Asn-Gln-Tyr-Val-Orn-Leu-) cyclic, where the indicated amino acids are the unusual D-isomer and Orn is the unusual amino acid ornithine. In tyrocidines B, C, and D, the aromatic residues at positions three, four, and seven are gradually replaced by tryptophan (Trauger et al. 2000). Tyrocidine is produced by a functional enzyme complex consisting of three NRPS, TycA, TycB, and TycC. (Mootz and Marahiel 1997).

Other non-ribosomal peptides of present interest include protease or proteasome inhibitor peptides, such as epoxyketones, e.g., eponemycin and epoxomicin, as well as carmaphycin, TMC-89A, macyranones, clarepoxcins, and landepoxcins; syrbactins, such as syringolins, e.g., syringolin A, glidobactins, e.g., glidobactin A, and cepafungins. See e.g., Kaysser 2019. Also of interest are gramicidins, e.g., Gramicidin S. See, e.g., Ogasawara and Dairi 2018.

In specific embodiments, the NRPS that may be utilised in the present methods include but are not limited to bacterial enzymes, such as Pseudomonas, Streptomyces, or Bacillus (e.g., Brevibacillus) enzymes. Exemplifications of these are NRPS that produce siderophore peptides or antibiotic peptides, and NRPS that produce protease or proteasome inhibitors. Examples include but are not limited to: Epn enzymes such as EpnG, and Epx enzymes such as EpxD, Syl enzymes such as SylC and SylD, Glb enzymes such as GlbC and GlbF, Grs enzymes such as GrsA and GrsB, as well as Pvd enzymes such as PvdJ and PvdD, and Tyc enzymes such as TycA, TycB, and TycC, and any variants of these, as described herein.

Modified NRPS enzymes, as well as modified NRPS polypeptides, modified NRPS domains, and modified NRPS modules are particularly noted. Of interest are NRPS polypeptides with a modified A domain, and in particular, a modified A domain comprising a linker region or part thereof, and downstream of this, a substrate binding pocket sequence of an A domain, and, optionally, an additional linker sequence downstream to the substrate binding pocket sequence of the A domain. As specific exemplifications, the modified A domain may be a modified A domain from one or more of the NRPS enzymes noted herein. Modified polynucleotides that encode the modified amino acid sequences are also noted.

In specific embodiments, NRP of the present disclosure include but are not limited to bacterial peptides, such as Pseudomonas, Streptomyces, or Bacillus (e.g., Brevibacillus) peptides. Exemplifications of these are siderophore non-ribosomal peptides, antibiotic non-ribosomal peptides, anticancer non-ribosomal peptides, and non-ribosomal peptides with protease or proteome inhibitory activity. Exemplifications of these are pyoverdines, tyrocidines, peptidyl-epoxyketones, syrbactins, and gramicidins. Examples include but are not limited to: PvdI, II, III, tyrocidine A, B, C, D, eponemycin, epoxomicin, syringolin, glidobactin, and Gramicidin S, and any variants thereof, as described herein. Of particular interest are NRP produced with a modified NRPS, a modified NRPS module, or a modified NRPS domain, as detailed in this description.

### Related Peptides, Polypeptides, and Polynucleotides

In addition to the sequences noted herein, the methods of the invention may be used to obtain modified peptide, polypeptide, and polynucleotide sequences. In one embodiment, the invention utilises modified NRPS polynucleotides and polypeptides, for example, fragments or sequence variations as described herein. Modifications of NRPS polypeptides, including modified NRPS domains, and modified NRPS modules, are specifically encompassed by the present disclosure. Modified polynucleotides encoding these sequences are also encompassed, as are modified NRP produced by these NRPS polypeptides.

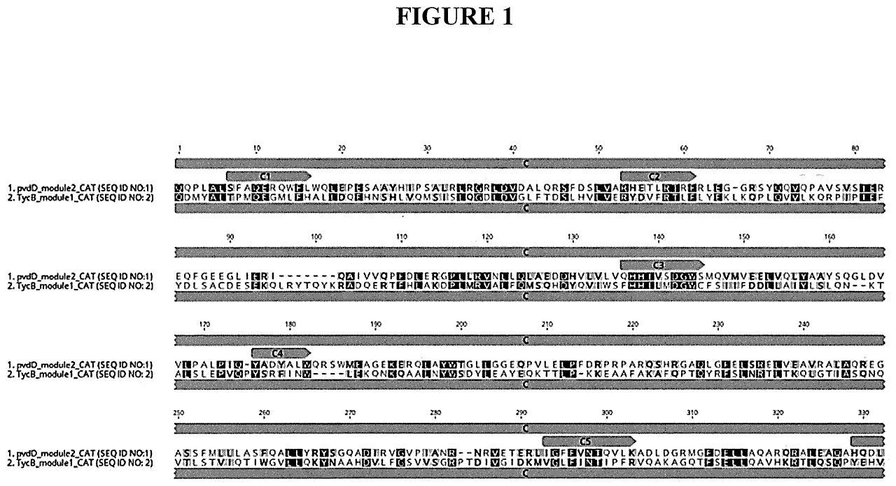

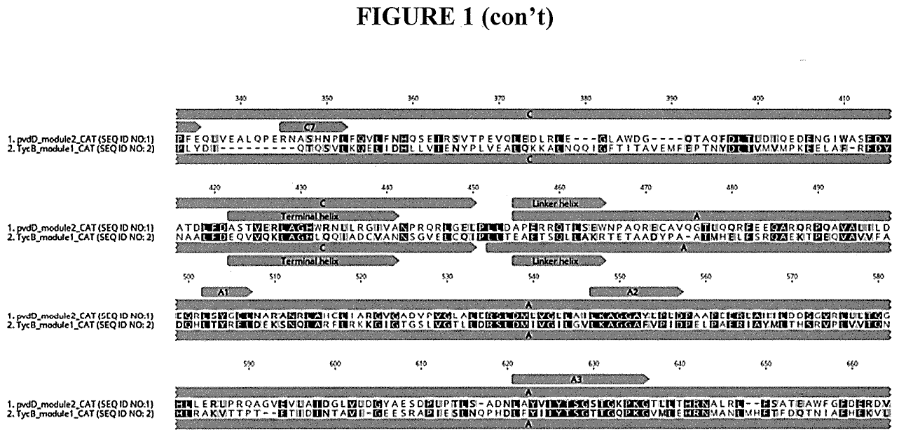

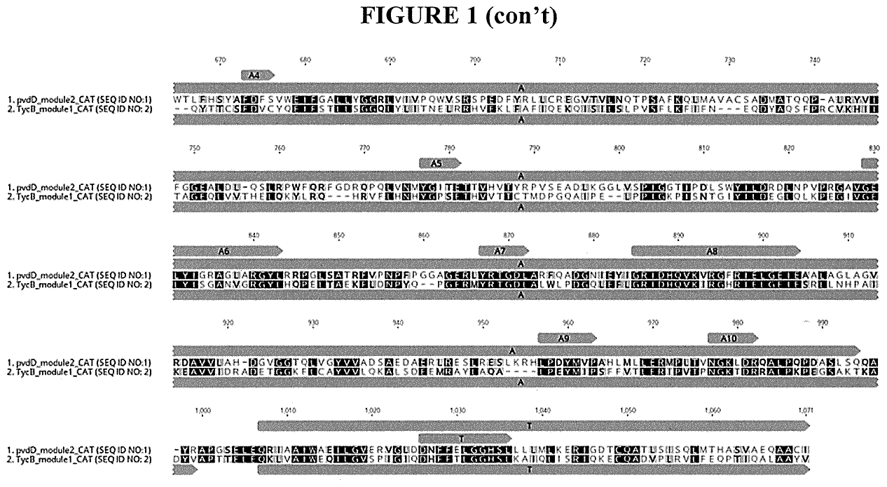

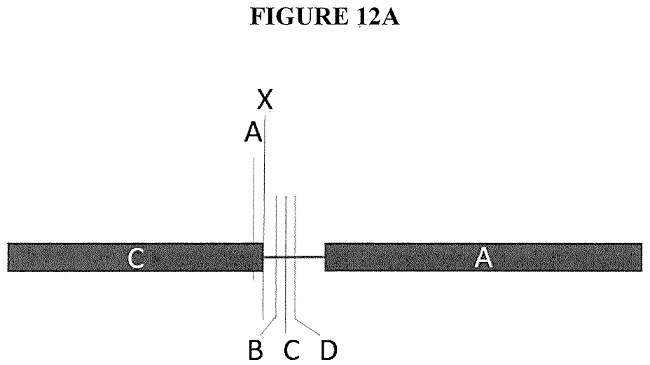

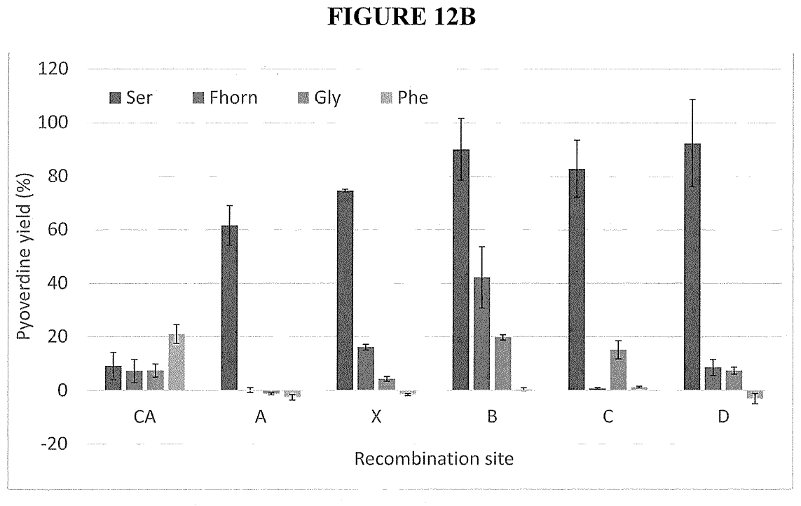

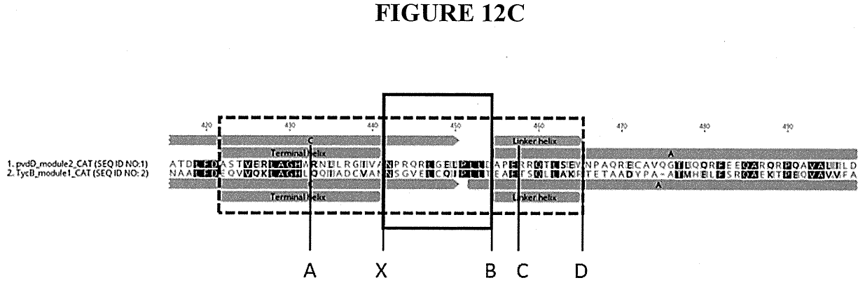

As demonstrated herein, the inventors have found that functional recombinant NRPS modules can be created by substituting an A domain from one NRPS module into another NRPS module, utilising favourable recombination sites. In particular, recombination sites can be utilised within the alpha-helix of the C domain (referred to as the terminal helix), or within the helix situated between the A domain and the C domain (referred to as the linker helix; Tanovic et al. 2008), or within the sequence that separates the two helices. The most preferred recombination sites are within the region spanning from the C-terminus of the terminal helix of the C domain to the N-terminus of the 11-residue helix within the linker region of the A domain. FIG. 1 shows the amino acid sequences that constitute the terminal helix and the linker helix, as depicted for the C-A-T domains from the second module of PvdD (SEQ ID NO: 1) and the first module of TycB (SEQ ID NO: 2). FIG. 12C shows the different recombination points that have been used to generate functional recombinant PvdD enzymes that were able to produce modified pyoverdines, as presently disclosed.

In accordance with the current findings, preferred recombination sites will reside within the nucleotide sequence that encodes, inclusively, the terminal helix of the C domain through to the linker helix of the A domain. To identify the terminal helix and the linker helix within an NRPS module that comprises a C domain joined to an A domain, the primary amino acid sequence can be analysed by standard methods. In particular, a secondary structure prediction tool may be used, such as YASPIN (Lin et al, 2005). Alternatively, equivalent regions can be determined using sequence alignment to a previously analysed module. For example, the present disclosure demonstrates that the sequence alignment in FIG. 12C can be used to locate the equivalent regions between PvdD module 2 and TycB module 1. In this way, it will be possible to substitute an A domain in any of the NRPS polypeptides set out herein.

The present disclosure therefore encompasses a non-naturally occurring NRPS polypeptide, for example, a non-naturally occurring NRPS module, which comprises in an N-terminal to C-terminal direction: (1) an amino acid sequence from a first NRPS module, e.g., comprising a C-domain from the C1 motif to the C7 motif, and (2) an amino acid sequence from a second NRPS module, e.g., comprising an A domain or a fragment thereof. The sequence of the second NRPS module may include an A domain binding pocket. It is expected that inclusion of an A domain binding pocket can be particularly advantageous. In particular, the new A domain binding pocket can be one that activates a different amino acid. For example, the binding pocket of the new A domain may differ from the original A domain by 1 to 10 amino acids. The one or more altered amino acids may be one or more of the eight amino acids that determine the specificity of a particular A domain, as described herein. This may be determined by the Stachelhaus code, or other suitable means. In certain aspects, the A domains can be substituted between NRPS polypeptides that have relatively low sequence identity. For example, lower levels of sequence identity may be found between the C domains of the two NRPS polypeptides, between the linker regions of the NRPS polypeptides, or between the A domains of the NRPS polypeptides. The sequence of the second NRPS module may include an optional additional C-terminal sequence. In particular, it may be advantageous to include a C-terminal sequence comprising a domain from another NRPS module. For example, this can be a domain from the first NRPS module or from a different NRPS module altogether.

As an N-terminal junction, the sequence of the second NRPS module may begin at a site within the terminal helix of the C domain of the first NRPS module, at a site within the linker helix of the A domain of the first NRPS module, or at a site between the terminal helix of the C domain and linker helix of the A domain of the first NRPS module, inclusive. As a C-terminal junction, the sequence of the second NRPS module may end at a site after the A10 motif of the first NRPS module, whether it lies within the A domain or the T domain. It will be understood that the junctions of the second NRPS module may be altered as desired.

Therefore, in various embodiments, the amino acid sequence from the second NRPS module may begin (i.e., N-terminal junction) at a site positioned within the terminal helix of the C domain of the first NRPS module, at a site positioned within the linker helix of the A domain of the first NRPS module, or at a site positioned in the region between these helices. Preferably, the amino acid sequence from the second NRPS module begins at a site positioned between the terminal helix of the C domain and the linker helix of the A domain of the first NRPS module. The region encompassing the terminal helix of the C domain to the linker helix of the A domain may comprise, e.g., at least 10 amino acids, at least 11 amino acids, at least 12 amino acids, at least 13 amino acids, at least 14 amino acids, or at least 15 amino acids. As one example, the amino acid sequence from the second NRPS module may begin at a position immediately following the C-terminus of the terminal helix of the C domain of the first NRPS module. As one other example, the amino acid sequence from the second module may begin at a position immediately preceding the N-terminus of the linker helix of the A domain of the first NRPS module.

In additional embodiments, the amino acid sequence from the second NRPS module may end (i.e., C-terminal junction) at a site preceding the first helix of the T domain of the first NRPS module. As one example, the amino acid sequence from the second NRPS module comprises a sequence that ends at a site in a region encompassing: the residue immediately following the A domain binding pocket to 20 residues following the A10 motif of the first NRPS module. As one other example, the amino acid sequence from the second NRPS module comprises a sequence that ends at a site in a region encompassing: the residue immediately following the A domain binding pocket to 10 residues following the A10 motif of the first NRPS module. In particular, it may be advantageous to utilise an approach where the A domain is substituted without any of the corresponding T domain. This approach can be used to makes scaling up easier. By avoiding modification of the T domain, this can allow the enzyme to pass the substrate to the C-terminal domain (Calcott and Ackerley, 2015; Owen et al, 2016; Linne et al, 2001; Strieker et al, 2010). This approach also keeps the substituted region as small as possible, which in general, reduces costs and makes polynucleotide manipulations easier.

It will be understood that any of the exemplary downstream junctions can be utilised with any of the exemplary upstream junctions, in accordance with the present disclosure. In certain embodiments, the first NRPS module and the second NRPS module have different substrate specificity. While not strictly necessary, it may be desirable that the first NRPS module and the second NRPS module share reduced levels of sequence identity. For example, the amino acid sequence from the second NRPS module may share less than 40%, less than 50%, less than 60% or less than 70% sequence identity to the equivalent amino acid sequence from the first NRPS module. In the same way, the amino acid sequence from the first NRPS module may share less than 40%, less than 50%, less than 60% or less than 70% sequence identity to the equivalent amino acid sequence from the second NRPS module. In addition, the A domain binding pocket of the second module may differ from the A domain binding pocket of the first module by 1 or more amino acids. For example, the A domain binding pocket of the second module may differ from the A domain binding pocket of the first module by 2 or more, 3 or more, 4 or more, 5 or more, 6 or more, 7 or more, or 8 amino acids.

It may be desirable to include an optional C-terminal sequence as part of the amino acid sequence from the second NRPS module. For example, as an optional C-terminal sequence, the sequence from the second NRPS module may include a sequence from the first NRPS module. In this case, the A domain may be substituted on its own into a different module, i.e., the C and T domains are from the first module and the A domain are from the second module. Alternatively, as an optional C-terminal sequence, the sequence from the second NRPS module may include a sequence from a third NRPS module. While the experiments set out herein use an LCL domain and a DCL domain, the present disclosure also allows for A domain substitutions juxtaposed to other types of C domains. Various functional subtypes of the C domain exist. For example, an LCL domain catalyses a peptide bond between two L-amino acids, a DCL domain links an L-amino acid to a growing peptide ending with a D-amino acid, a Starter C domain acylates the first amino acid with a β-hydroxy-carboxylic acid (typically a β-hydroxyl fatty acid), and heterocyclisation (Cyc) domains catalyse both peptide bond formation and subsequent cyclization of cysteine, serine or threonine residues. Further to this, dual E/C domains catalyse both epimerization and condensation (Rausch et al. 2007).

While the above noted embodiments have been discussed in terms of amino acid sequences, it should be acknowledged that the NRPS domains, modules, helices, junction sites, N-terminal sites, and C-terminal sites can be understood in terms of the corresponding nucleotide sequences. In particular, favourable recombination sites can be determined from the description noted above and elsewhere herein, so as to construct the non-naturally occurring polypeptide of the present disclosure.

In addition the particular nucleotide and amino acid sequences set out herein, variant sequences may also be utilised. In various embodiments, polynucleotide variants encompass naturally occurring, recombinantly, and synthetically produced polynucleotides. As exemplifications, variant polynucleotide sequences exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, and at least 99% identity to a sequence of the present disclosure. In the same way, a polynucleotide encoding an NRPS, an NRPS module, an NRPS domain, an NRPS domain helix, or an NRPS domain binding pocket may be modified to include the above noted levels of sequence identity.

As a variant polynucleotide sequence, a fragment of a polynucleotide sequence includes a subsequence of contiguous nucleotides. In one embodiment, the polynucleotide fragment allows expression of at least a portion of an NRPS, e.g., expression of one or more functional domain of the polypeptide. Specifically noted are polynucleotides and polynucleotide fragments encoding A domains, fragments of A domains, and any modified A domains, as described herein.

Variant polynucleotides include polynucleotides that differ from the disclosed sequences but that, as a consequence of the degeneracy of the genetic code, encode a polypeptide having similar activity to a polypeptide encoded by a disclosed polynucleotide. A sequence alteration that does not change the amino acid sequence of the polypeptide is termed a silent variation. Except for ATG (methionine) and TGG (tryptophan), other codons for the same amino acid may be changed by art recognised techniques, e.g., to optimise codon expression in a particular host organism.

For polynucleotides, sequence identity may be found over a comparison window of at least 1500 nucleotide positions, at least 2000 nucleotide positions, at least 2500 nucleotide positions, at least 3000 nucleotide positions, at least 3500 nucleotide positions, at least 3800 nucleotide positions, or over the entire length of a polynucleotide used according to a method of the invention. For a polynucleotide encoding an NRPS module, an NRPS domain, an NRPS domain helix, or an NRPS domain binding pocket, shorter regions may be compared, for example, at least 50 nucleotide positions, at least 100 nucleotide positions, at least 200 nucleotide positions, at least 300 nucleotide positions, at least 400 nucleotide positions, at least 500 nucleotide positions, at least 600 nucleotide positions, at least 700 nucleotide positions, at least 800 nucleotide positions, at least 900 nucleotide positions, or at least 1000 nucleotide positions.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al. 1990).

Polynucleotide sequence identity and similarity can be determined in the following manner. The subject polynucleotide sequence is compared to a candidate polynucleotide sequence using sequence alignment algorithms and sequence similarity search tools such as in GenBank, EMBL, Swiss-PROT and other databases. Nucleic Acids Res 29:1-10 and 11-16, 2001 provides examples of online resources.

In various embodiments, polypeptide variants encompass naturally occurring, recombinantly, and synthetically produced polypeptides. As exemplifications, variant polypeptide sequences exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence of the present disclosure. In the same way, a polypeptide sequence of an NRPS, an NRPS module, an NRPS domain, an NRPS domain helix, or an NRPS domain binding pocket may be modified to include the above noted levels of sequence identity.

As a variant polypeptide sequence, a fragment of a polypeptide sequence includes a subsequence of contiguous amino acids. In one embodiment, a polypeptide fragment is a functional fragment, i.e., a fragment capable of binding or other biological activity. For example, an NRPS polypeptide fragment may be capable of producing a particular NRP. In a particular embodiment, the polypeptide fragment may include at least one functional domain. For example, for an NRPS polypeptide, a fragment would include an A domain, an A domain fragment, or a modification thereof, as described herein.

As to polypeptide variants, an amino acid sequence may differ from a polypeptide disclosed herein by one or more conservative amino acid substitutions, deletions, additions or insertions which do not affect the biological activity of the peptide. Conservative substitutions typically include the substitution of one amino acid for another with similar characteristics, e.g., substitutions within the following groups: glycine, alanine; valine, isoleucine, leucine; aspartic acid, glutamic acid; asparagine, glutamine; serine, threonine; lysine, arginine; and phenylalanine, tyrosine. Non-conservative substitutions will entail exchanging a member of one of these classes for a member of another class.

Other variants include peptides with modifications which influence peptide stability. Such analogues may contain, for example, one or more non-peptide bonds (which replace the peptide bonds) in the peptide sequence. Also included are analogues that include residues other than naturally occurring L-amino acids, e.g. D-amino acids or non-naturally occurring synthetic amino acids, e.g. beta or gamma amino acids and cyclic analogues.

Substitutions, deletions, additions, or insertions may be made by mutagenesis methods known in the art. A skilled worker will be aware of methods for making phenotypically silent amino acid substitutions. See, for example, Bowie et al. 1990. A polypeptide may be modified during or after synthesis, for example, by biotinylation, benzylation, glycosylation, phosphorylation, amidation, by derivatisation using blocking/protecting groups and the like. Such modifications may increase stability or activity of the polypeptide.

For polypeptides, sequence identity may be found over a comparison window of at least 600 amino acid positions, at least 700 amino acid positions, at least 800 amino acid positions, at least 900 amino acid positions, at least 1000 amino acid positions, at least 1100 amino acid positions, at least 1200 amino acid positions, or over the entire length of a polypeptide used in or identified according to a method of the invention. For a polypeptide comprising an NRPS module, an NRPS domain, an NRPS domain helix, or an NRPS domain binding pocket, shorter regions may be compared, for example, at least 8 amino acid positions, at least 10 amino acid positions, at least 20 amino acid positions, at least 30 amino acid positions, at least 40 amino acid positions, at least 50 amino acid positions, at least 60 amino acid positions, at least 70 amino acid positions, at least 80 amino acid positions, at least 90 amino acid positions, or at least 100 amino acid positions.

Polypeptide variants also encompass those that exhibit a similarity to one or more of the specifically identified sequences that is likely to preserve the functional equivalence of those sequences and which could not reasonably be expected to have occurred by random chance. For polynucleotides and polypeptides, exemplary sequence alignment platforms include but are not limited to: homology alignment algorithms (Needleman and Wunsch (1970) J Mol Biol 48: 443); local homology algorithms (Smith and Waterman (1981) Adv Appl Math 2: 482); searches for similarity (Pearson and Lipman (1988) PNAS USA 85: 2444). In specific embodiments, the BLAST algorithm may be used (Altschul et al. (1990) J Mol Biol 215: 403-410; Henikoff and Henikoff. (1989) PNAS USA 89: 10915; Karlin and Altschul (1993) PNAS USA 90: 5873-5787). Software for performing BLAST analyses is publicly available through the National Center for Biotechnology Information. Other examples of alignment software include GAP, BESTFIT, FASTA, PILEUP, and TFASTA provided by Wisconsin Genetics Software Package (Genetics Computer Group), and CLUSTAL programs such as ClustalW, ClustalX, and Clustal Omega (see, e.g., Thompson et al. (1994) Nuc Acids Res 22: 4673-4680).

### Expression of NRPS Polypeptides and Production of NRP

In one embodiment of the present disclosure, the NRPS polypeptide (e.g., a modified NRPS polypeptide) is expressed using a nucleic acid construct. For example, an NRPS construct may be used, i.e., a nucleic acid expression construct that comprises a polynucleotide sequence that encodes an NRPS polypeptide operatively linked to a promoter that allows expression of the polynucleotide sequence to form the NRPS polypeptide. Preferably, the NRPS polypeptide that is employed produces a protease inhibitor or proteasome inhibitor, a siderophore peptide, an anticancer peptide, or antibiotic peptide, or a functional variant of the polypeptide may be employed.

An expression cassette may be used to include the necessary elements that permit the transcription of a polynucleotide molecule that has been cloned or inserted into the construct. Optionally, the expression cassette may comprise some or all of the necessary elements for translating the transcript produced from the expression cassette into a polypeptide. An expression cassette may include NRPS coding regions. It may also include any necessary noncoding regions.

The NRPS construct may be a construct for expression the appropriate NRPS polypeptide, as set out herein, or other peptide-producing NRPS polypeptide, or any functional variants thereof. The construct may be a nucleic acid expression construct comprising a polynucleotide sequence encoding the NRPS polypeptide operatively linked to a promoter that allows expression of the polynucleotide sequence.

In accordance with the present invention, the activation of the NRPS polypeptide or its functional variant can be carried out prior to or following isolation of the NRPS polypeptide or its functional variant, i.e., pre-isolation activation or post-isolation activation. In addition, the NRPS polypeptide or its functional variant may be activated in vitro prior to incubation with a test sample, or the NRPS polypeptide or its functional variant may be activated in vivo and isolated prior to incubation with a test sample.

The polynucleotide sequence encoding the NRPS polypeptide may be any suitable NRPS polynucleotide sequence from any organism. Preferably the organism is a bacterial cell or strain. Exemplifications include but are not limited to: Pseudomonas, Streptomyces, and Bacillus (e.g., Brevibacillus) strains, for example, P. aeruginosa, P. syringae, P. putida, P. fluorescens, and B. brevis, as well as other bacterial strains, as described in detail herein. The polynucleotide sequence encoding the NRPS polypeptide may be a naturally occurring (i.e., wild-type) or modified polynucleotide sequence. For example, a wild-type or a modified polynucleotide sequence for one or more NRPS domains may be used. In particular, the polynucleotide sequence encoding the NRPS module may be a wild-type or modified polynucleotide sequence, as described herein.

In one embodiment, a construct is made by cloning a polynucleotide sequence encoding a wild-type or modified polypeptide as above into an appropriate vector. An appropriate vector is any vector that comprises a promoter operatively linked to the cloned, inserted polynucleotide sequence that allows expression of the polypeptide from the vector. A skilled worker appreciates that different vectors may be employed in the methods of the invention. In addition methods for constructing vectors, including the choice of an appropriate vector, and the cloning and expression of a polynucleotide sequence inserted into an appropriate vector as described above is believed to be within the capabilities of a person of skill in the art (Sambrook et al. 2003).

Preferably, the expressed NRPS polypeptide comprises a functional NRPS module, or a functional variant thereof. Expression may be inducible, for example, with IPTG. Similar approaches may be used for the NRPS polypeptides disclosed herein, and any functional variants thereof. The person of skill in the art recognises that there are also many suitable alternative expression systems available that may be used in the methods of the invention to express an NRPS polypeptide.

Preferably, expression is in a suitable host cell or strain. In one embodiment, the host cell or strain may be a cell or strain of E. coli. Particularly of interest is the BAP1 strain of E. coli or any variant of this strain (Pfeifer et al. 2001). Alternatively, the expression vector is chosen to allow inducible expression in a non-E. coli host cell or strain. Expression may also be obtained using in vitro expression systems; such systems are well known in the art.

In one embodiment, multiple NRPS polypeptides are co-expressed in the same host cell or strain. To achieve expression within the same host cell or strain, the nucleotide sequences encoding the NRPS polypeptides may be cloned into suitable, separate expression vectors. Suitable vectors may have the same or compatible origins of replication in order to be stably maintained in the same host cell or strain. Preferably, at least one construct encodes an NRPS module or a functional variant thereof.

In another embodiment, one or more polynucleotide sequences encoding a NRPS polypeptide may be integrated into the chromosome of an appropriate host organism as described herein, to produce a strain useful in accordance with the present disclosure. In one embodiment, an NRPS construct comprises a nucleotide sequence encoding an NRPS polypeptide and a suitable regulatory promoter that is integrated into the chromosome of E. coli or other host organism in an appropriate orientation to allow expression of the polypeptide in the cell.

In one particular embodiment, a construct encoding an NRPS module is integrated into a host cell. For example, an NRPS construct may be integrated and then expressed in vivo. The constructs may allow co-expression of wild-type polypeptides or functional variants. Thus, in a specific embodiment, a construct that encodes an NRPS module is expressed in a host cell or strain.

In specific embodiments of the present disclosure, the expressed NRPS polypeptide may be isolated using various biochemical techniques. These techniques include but are not limited to filtration, centrifugation, and various types of chromatography, such as ion-exchange, affinity, hydrophobic interaction, size exclusion, and reverse-phase chromatography. In one particular embodiment, Ni-NTA affinity chromatography is used. As exemplifications, the polypeptides may be linked to a solid substrate such as beads, filters, fibers, paper, membranes, chips, and plates such as multiwell plates. The polypeptides may also be prepared as a polypeptide conjugates in accordance with known methods.

In particular embodiments, the present disclosure provides polynucleotide libraries that include NRPS nucleic acids. For example, a polynucleotide library may include at least 15, at least 25, at least 50, at least 100, at least 200, at least 300, at least 400, at least 500, at least 600, at least 700, at least 800, at least 900, or at least 1000 NRPS nucleic acids. Libraries of NRPS polynucleotides, and specifically variant NRPS polynucleotides, may be generated using standard methods. As exemplifications, nucleic acid libraries may be generated to include a plurality of NRPS polynucleotides with modified NRPS modules or modified NRPS domains. For example, a nucleic acid library may include NRPS polynucleotides with A domain substitutions, i.e., domain swap libraries. In addition, libraries of NRPS polynucleotides may be generated using random mutagenesis of one or more domains (e.g., A domain mutagenesis), for example, error prone PCR may be utilised (see, e.g., Beaudry and Joyce (1992) Science 257: 635 and Bartel and Szostak (1993) Science 261: 1411). Alternative means for mutagenesis may be used, for example, chemical mutagens, radiation, amongst others. Commercial kits are also available, e.g., GeneMorph® II EZClone domain mutagenesis kit (Agilent) and Diversify™ PCR random mutagenesis kit (Clontech Laboratories, Inc). The library may be provided as a mixture of polynucleotides, or may be provided via a host cell or strain.

As one embodiment of the present disclosure, a kit is provided which includes one or more NRPS polynucleotide or polypeptide. The one or more NRPS polynucleotide or polypeptide may be a modified component as described herein. The one or more polynucleotide or polypeptide may be provided in one or more containers in the kit. Additional components may also be provided with the kit, for example, one or more components to obtain expression, or one or more components to measure activity, which are intended for use with the polynucleotide(s) or polypeptide(s). Optionally, instructions may be provided with the kit, as well as any other item, such as any number of containers, labels, or measurement tools. The one or more polynucleotide or polypeptide of the kit may be provided as isolated components, or as mixtures, or may be provided via a host cell or strain.

### Host Cells and Strains

As disclosed herein, methods of production for an NRPS polypeptide (e.g., a modified NRPS polypeptide) are provided, and methods of production of peptides from a non-naturally occurring NRPS polypeptide are also provided. Host cells and their use for such production are set out in detail in this description. By use of a host cell comprising an NRPS polypeptide, this allows production of compounds by fermentation. Previously, researchers have had to purify NRPS enzymes and then attempt to use the purified enzymes in in vitro systems. An acknowledged goal for NRPS polypeptides is fermentation, i.e. in vivo production, and the methods of the present disclosure provide for this.

The expression of an NRPS polypeptide (e.g., a modified NRPS polypeptide) may be carried out in vitro or in vivo. In vivo expression may be carried out in a suitable host cell or strain. A suitable host cell or strain may be any suitable prokaryotic or eukaryotic cell in which a NRPS polypeptide, or any functional variants thereof, may be expressed. A suitable host cell or strain may be any suitable prokaryotic or eukaryotic cell in which the NRPS polypeptide may be expressed wherein the NRPS polypeptide is not activated in the cell by any endogenous activity of the cell. The suitable host cell or strain may be a bacterial cell or strain. In particular embodiments, eukaryotic cells or strains may be used.

Introduction of an NRPS construct into an appropriate host cell or strain may be achieved using any of a number of available standard protocols and/or as described herein as known and used in the art (Sambrook et al. 2003). Preferably, the NRPS construct is a construct for a synthetase polypeptide as set out herein. Preferably, the construct is inserted into an appropriate host cell or strain. Such insertion may be achieved using any of a number of available standard transformation or transduction protocols as known and used in the art (Sambrook et al. 2003).

In certain embodiments, the host cell or strain expresses a NRPS polypeptide that can be activated by a PPTase. In one embodiment, the host cell or strain is a fungal or bacterial, preferably bacterial, host cell or strain, but not limited thereto. Preferably, the bacterial cell or strain is a Gram negative bacterial cell or strain. Preferably, the bacterial cell or strain is a cell or strain of E. coli. For industrial applications, the host strain may be a Bacillus (e.g., Brevibacillus), Streptomyces, or Pseudomonas strain, or another bacterial strain as set out herein, or any functional variant thereof.

In one embodiment, the expressed polypeptide (e.g., NRPS polypeptide) is an exogenous polypeptide in the host cell or strain expressed from a construct according to the invention, but not limited thereto. Alternatively, the polypeptide is expressed from the genome of the host cell or strain. In this embodiment, the polypeptide may be endogenous or exogenous, naturally occurring or non-naturally occurring with respect of the host cell or strain. In one particular embodiment, a single host organism could be modified to allow expression of multiple NRPS polypeptides in the cell, including multiple modified NRPS modules, to maximise production of the peptide product.

By way of non-limiting example, the NRPS polypeptide may be an exogenous polypeptide expressed from an NRPS expression construct. Preferably the NRPS so expressed is a synthetase polypeptide as set out herein, or a functional variant thereof. In this embodiment, the NRPS polypeptide is an exogenous NRPS polypeptide that synthesizes a protease or proteasome inhibitor, a siderophore, an anticancer peptide, or an antibiotic peptide. Preferably, the synthesised product is an NRP as set out herein, or a variant thereof. The synthesis of an NRP may be carried out in vitro or in vivo. In vivo expression may be carried out in a suitable host cell or strain. A suitable host cell or strain may be any suitable prokaryotic or eukaryotic cell in which an NRP, or any functional variants thereof, may be synthesised. A suitable host cell or strain may be any suitable prokaryotic or eukaryotic cell in which the peptide may be synthesised wherein the corresponding NRPS polypeptide is not activated in the cell by any endogenous activity of the cell. The suitable host cell or strain may be a bacterial cell or strain. In particular embodiments, eukaryotic cells or strains may be used.

Host cells and strains useful in the invention are not limited to strains of E. coli or the other strains described herein, such as Pseudomonas, Streptomyces, and Bacillus (e.g., Brevibacillus) strains, for example, P. aeruginosa (e.g., P. aeruginosa PAO1), P. syringae (e.g., P. syringae pv. phaseolicola 1448A), P. putida (e.g., P. putida KT2440), P. fluorescens, and B. brevis. Numerous alternative host organisms may be useful in the methods according to the invention, wherein each cell or strain may provide a different or additional benefit or utility. The choice of an appropriate host strain will affect choice of construct used based on the genetic makeup of the host. A key reason for using different host strains is that not all proteins can be expressed effectively in some strains (e.g., E. coli strains) due to promoter inactivity, codon bias, protein insolubility, or other factors. Therefore, the use of different host strains provides alternative hosts suitable for use in production of any polypeptide or peptide of interest. Cell free expression systems and cell free synthesis systems may also be used in accordance with standard methodology.

### Sequence Information

The nucleotide and amino acid sequences of the present disclosure are set out below. A brief description of each sequence is also provided in the table, below.

SEQ ID NO: 1 is an amino acid sequence of the CAT-domains from the second module of PvdD
SEQ ID NO: 2 is an amino acid sequence of the CAT-domains from the first module of PvdJ
SEQ ID NO: 3 is the nucleotide sequence of the plasmid pUCP22:pBAD
SEQ ID NO: 4 is the nucleotide sequence of the plasmid pUCBAD-SMC
SEQ ID NO: 5 is the nucleotide sequence of the plasmid pDEC-Lys
SEQ ID NO: 6 is the nucleotide sequence of the plasmid pDEC-Thr
SEQ ID NO: 7 is the nucleotide sequence of the plasmid pTRN
SEQ ID NO: 8 is the nucleotide sequence used to substitute the C domain from the first
module of PvdJ into the second module of PvdD
SEQ ID NO: 9 is the translation of SEQ ID NO: 8 having the residues substituted into PvdD
underlined.
SEQ ID NO: 10 is the nucleotide sequence used to substitute the C domain from the first
module of PvdD back into the second module of PvdD
SEQ ID NO: 11 is the translation of SEQ ID NO: 10
SEQ ID NO: 12 is the nucleotide sequence used to substitute region 1 of the C domain from
the first module of PvdJ into the second module of PvdD
SEQ ID NO: 13 is the translation of SEQ ID NO: 12 having the residues substituted into
PvdD underlined.
SEQ ID NO: 14 is the nucleotide sequence used to substitute region 2 of the C domain from
the first module of PvdJ into the second module of PvdD
SEQ ID NO: 15 is the translation of SEQ ID NO: 14 having the residues substituted into
PvdD underlined.
SEQ ID NO: 16 is the nucleotide sequence used to substitute region 3 of the C domain from
the first module of PvdJ into the second module of PvdD
SEQ ID NO: 17 is the translation of SEQ ID NO: 16 having the residues substituted into
PvdD underlined.
SEQ ID NO: 18 is the nucleotide sequence used to substitute regions 2 and 3 of the C
domain from the first module of PvdJ into the second module of PvdD
SEQ ID NO: 19 is the translation of SEQ ID NO: 18 having the residues substituted into
PvdD underlined.
SEQ ID NO: 20 is the nucleotide sequence used to substitute regions 1 and 3 of the C
domain from the first module of PvdJ into the second module of PvdD
SEQ ID NO: 21 is the translation of SEQ ID NO: 20 having the residues substituted into
PvdD underlined.
SEQ ID NO: 22 is the nucleotide sequence used to substitute regions 1 and 2 of the C
domain from the first module of PvdJ into the second module of PvdD
SEQ ID NO: 23 is the translation of SEQ ID NO: 22 having the residues substituted into
PvdD underlined.
SEQ ID NO: 24 is the nucleotide sequence used to substitute 6 residues of the C domain
from the first module of PvdJ into the second module of PvdD
SEQ ID NO: 25 is the translation of SEQ ID NO: 24 having the residues substituted into
PvdD underlined.
SEQ ID NO: 26 is the nucleotide sequence used to substitute 12 residues of the C domain
from the first module of PvdJ into the second module of PvdD
SEQ ID NO: 27 is the translation of SEQ ID NO: 26 having the residues substituted into
PvdD underlined.
SEQ ID NO: 28 is the nucleotide sequence used to substitute the loop residues of the C
domain from the first module of PvdJ into the second module of PvdD
SEQ ID NO: 29 is the translation of SEQ ID NO: 28 having the residues substituted into
PvdD underlined.
SEQ ID NO: 30 is the nucleotide sequence used to substitute the 6 residues plus the loop
residues of the C domain from the first module of PvdJ into the second module of PvdD
SEQ ID NO: 31 is the translation of SEQ ID NO: 30 having the residues substituted into
PvdD underlined.
SEQ ID NO: 32 is the nucleotide sequence used to substitute the 6 residues plus the edge
loop residues of the C domain from the first module of PvdJ into the second module of
PvdD
SEQ ID NO: 33 is the translation of SEQ ID NO: 32 having the residues substituted into
PvdD underlined.
SEQ ID NO: 34 is the nucleotide sequence used to substitute the 12 residues plus the loop
residues of the C domain from the first module of PvdJ into the second module of PvdD
SEQ ID NO: 35 is the translation of SEQ ID NO: 34 having the residues substituted into
PvdD underlined.
SEQ ID NO: 36 is the nucleotide sequence used to substitute the linker residues of the C
domain from the first module of PvdJ into the second module of PvdD
SEQ ID NO: 37 is the translation of SEQ ID NO: 36 having the residues substituted into
PvdD underlined.
SEQ ID NO: 38 is the nucleotide sequence used to substitute CA-domains from the first
module of PvdJ into the second module of PvdD
SEQ ID NO: 39 is the translation of SEQ ID NO: 38 having the residues substituted into
PvdD underlined.
SEQ ID NO: 40 is the nucleotide sequence used to substitute CA-domains from Ser specific
module into the second module of PvdD
SEQ ID NO: 41 is the translation of SEQ ID NO: 40 having the residues substituted into
PvdD underlined.
SEQ ID NO: 42 is the nucleotide sequence used to substitute CA-domains from fhOrn
specific module into the second module of PvdD
SEQ ID NO: 43 is the translation of SEQ ID NO: 42 having the residues substituted into
PvdD underlined.
SEQ ID NO: 44 is the nucleotide sequence used to substitute the linker + A-domain from
a Lys specific module into the second module of PvdD
SEQ ID NO: 45 is the translation of SEQ ID NO: 44 having the residues substituted into
PvdD underlined.
SEQ ID NO: 46 is the nucleotide sequence used to substitute the linker + A-domain from
a Ser specific module into the second module of PvdD
SEQ ID NO: 47 is the translation of SEQ ID NO: 46 having the residues substituted into
PvdD underlined.
SEQ ID NO: 48 is the nucleotide sequence used to substitute the linker + A-domain from
fhOrn specific module into the second module of PvdD
SEQ ID NO: 49 is the translation of SEQ ID NO: 48 having the residues substituted into
PvdD underlined.
SEQ ID NO: 50 is the nucleotide sequence used to substitute the linker + A-domain from
CP008696.1.cluster009_A1 into the second module of PvdD
SEQ ID NO: 51 is the translation of SEQ ID NO: 50 having the residues substituted into
PvdD underlined.
SEQ ID NO: 52 is the nucleotide sequence used to substitute the linker + A-domain from
CP006852.1.cluster006_A4 into the second module of PvdD
SEQ ID NO: 53 is the translation of SEQ ID NO: 52 having the residues substituted into
PvdD underlined.
SEQ ID NO: 54 is the nucleotide sequence used to substitute the linker + A-domain from
CP011507.1.cluster002_A1 into the second module of PvdD
SEQ ID NO: 55 is the translation of SEQ ID NO: 54 having the residues substituted into
PvdD underlined.
SEQ ID NO: 56 is the nucleotide sequence used to substitute the linker + A-domain from
CP003041.1.cluster006_A2 into the second module of PvdD
SEQ ID NO: 57 is the translation of SEQ ID NO: 56 having the residues substituted into
PvdD underlined.
SEQ ID NO: 58 is the nucleotide sequence used to substitute the linker + A-domain from
APO 13068.l.cluster003_A1 into the second module of PvdD
SEQ ID NO: 59 is the translation of SEQ ID NO: 58 having the residues substituted into
PvdD underlined.
SEQ ID NO: 60 is the nucleotide sequence used to substitute the linker + A-domain from
CP010945.1.cluster006_A1 into the second module of PvdD
SEQ ID NO: 61 is the translation of SEQ ID NO: 60 having the residues substituted into
PvdD underlined.
SEQ ID NO: 62 is the nucleotide sequence used to substitute the linker + A-domain from
AM181176.4.cluster005_A2 into the second module of PvdD
SEQ ID NO: 63 is the translation of SEQ ID NO: 62 having the residues substituted into
PvdD underlined.
SEQ ID NO: 64 is the nucleotide sequence used to substitute the linker + A-domain from
CP000680.1.cluster003_A3 into the second module of PvdD
SEQ ID NO: 65 is the translation of SEQ ID NO: 64 having the residues substituted into
PvdD underlined.
SEQ ID NO: 66 is the nucleotide sequence used to substitute the linker + A-domain from
CP011972.1.cluster002_A4 into the second module of PvdD
SEQ ID NO: 67 is the translation of SEQ ID NO: 66 having the residues substituted into
PvdD underlined.
SEQ ID NOs: 68, 70, 72, 74, 76, 78, 80, 82, 84, 86, 88, 90 are the nucleotide sequences
used to substitute a small region of the Ser or Lys specific A-domains into the second
module of PvdD
SEQ ID NOs: 69, 71, 73, 75, 77, 79, 81, 83, 85, 87, 89, 91 are the translations of the
nucleotide sequences noted directly above having the residues substituted into PvdD
underlined.
SEQ ID NO: 92 is the nucleotide sequence used to substitute the Ser specific A-domain
with the upstream recombination point A into the second module of PvdD
SEQ ID NO: 93 is the translation of SEQ ID NO: 92 having the residues substituted into
PvdD underlined.
SEQ ID NO: 94 is the nucleotide sequence used to substitute the Ser specific A-domain
with the upstream recombination point B into the second module of PvdD
SEQ ID NO: 95 is the translation of SEQ ID NO: 94 having the residues substituted into
PvdD underlined.
SEQ ID NO: 96 is the nucleotide sequence used to substitute the Ser specific A-domain
with the upstream recombination point C into the second module of PvdD
SEQ ID NO: 97 is the translation of SEQ ID NO: 96 having the residues substituted into
PvdD underlined.
SEQ ID NO: 98 is the nucleotide sequence used to substitute the Ser specific A-domain
with the upstream recombination point D into the second module of PvdD
SEQ ID NO: 99 is the translation of SEQ ID NO: 98 having the residues substituted into
PvdD underlined.
SEQ ID NO: 100 is the nucleotide sequence used to substitute the fhOrn specific A-domain
with the upstream recombination point A into the second module of PvdD
SEQ ID NO: 101 is the translation of SEQ ID NO: 100 having the residues substituted into
PvdD underlined.
SEQ ID NO: 102 is the nucleotide sequence used to substitute the fhOrn specific A-domain
with the upstream recombination point B into the second module of PvdD
SEQ ID NO: 103 is the translation of SEQ ID NO: 102 having the residues substituted into
PvdD underlined.
SEQ ID NO: 104 is the nucleotide sequence used to substitute the fhOrn specific A-domain
with the upstream recombination point C into the second module of PvdD
SEQ ID NO: 105 is the translation of SEQ ID NO: 104 having the residues substituted into
PvdD underlined.
SEQ ID NO: 106 is the nucleotide sequence used to substitute the fhOrn specific A-domain
with the upstream recombination point D into the second module of PvdD
SEQ ID NO: 107 is the translation of SEQ ID NO: 106 having the residues substituted into
PvdD underlined.
SEQ ID NO: 108 is the nucleotide sequence of the plasmid pET28:ProC-TTe
SEQ ID NO: 109 is the nucleotide sequence used to substitute the A-domain from the first
module of TycB into the second module of PvdD
SEQ ID NO: 110 is the translation of SEQ ID NO: 109 having the residues substituted into
PvdD underlined.
SEQ ID NO: 111 is the nucleotide sequence used to substitute the A-domain from the sixth
module of TycC into the second module of PvdD
SEQ ID NO: 112 is the translation of SEQ ID NO: 111 having the residues substituted into
PvdD underlined.
SEQ ID NO: 113 is the nucleotide sequence used to substitute the A-domain from SrfA-C
into the second module of PvdD
SEQ ID NO: 114 is the translation of SEQ ID NO: 113 having the residues substituted into
PvdD underlined.
SEQ ID NO: 115 is the nucleotide sequence used to substitute the A-domain from
NZ_CP021920.1.cluster002_Phe into the second module of PvdD
SEQ ID NO: 116 is the translation of SEQ ID NO: 115 having the residues substituted into
PvdD underlined.
SEQ ID NO: 117 is the nucleotide sequence used to substitute the A-domain from
NZ_CP020028.1.cluster004_Leu into the second module of PvdD
SEQ ID NO: 118 is the translation of SEQ ID NO: 117 having the residues substituted into
PvdD underlined.
SEQ ID NO: 119 is the nucleotide sequence used to substitute the A-domain from
NZ_CM000756.1.cluster012_Leu into the second module of PvdD
SEQ ID NO: 120 is the translation of SEQ ID NO: 119 having the residues substituted into
PvdD underlined.
SEQ ID NO: 121 is the nucleotide sequence of the plasmid pACYC:PheATE
SEQ ID NO: 122 is an amino acid sequence of region 1 of the C domain from the second
module of PvdD
SEQ ID NO: 123 is an amino acid sequence of region 2 of the C domain from the second
module of PvdD
SEQ ID NO: 124 is an amino acid sequence of region 3 of the C domain from the second
module of PvdD
SEQ ID NO: 125 is an amino acid sequence of region 1 of the C domain from the second
module of PvdJ
SEQ ID NO: 126 is an amino acid sequence of region 2 of the C domain from the second
module of PvdJ
SEQ ID NO: 127 is an amino acid sequence of region 3 of the C domain from the second
module of PvdJ
SEQ ID NO: 128 is an amino acid sequence EpnG
SEQ ID NO: 129 is an amino acid sequence EpxD
SEQ ID NO: 130 is an amino acid sequence SylC
SEQ ID NO: 131 is an amino acid sequence SylD
SEQ ID NO: 132 is an amino acid sequence GlbC
SEQ ID NO: 133 is an amino acid sequence GlbF
SEQ ID NO: 134 is an amino acid sequence PvdJ
SEQ ID NO: 135 is an amino acid sequence PvdD
SEQ ID NO: 136 is an amino acid sequence TycA
SEQ ID NO: 137 is an amino acid sequence TycB
SEQ ID NO: 138 is an amino acid sequence TycC
SEQ ID NO: 139 is an amino acid sequence GrsA
SEQ ID NO: 140 is an amino acid sequence GrsB
SEQ ID NO: 141 is the nucleotide sequence used to substitute the C- and A-domains from a
Gly specifying module into the second module of PvdD
SEQ ID NO: 142 is the translation of SEQ ID NO: 141 having the residues substituted into
PvdD underlined
SEQ ID NO: 143 is the nucleotide sequence used to substitute the Gly specific A-domain
with the upstream recombination point A into the second module of PvdD
SEQ ID NO: 144 is the translation of SEQ ID NO: 143 having the residues substituted into
PvdD underlined
SEQ ID NO: 145 is the nucleotide sequence used to substitute the Gly specific A-domain
with the upstream recombination point X into the second module of PvdD
SEQ ID NO: 146 is the translation of SEQ ID NO: 145 having the residues substituted into
PvdD underlined
SEQ ID NO: 147 is the nucleotide sequence used to substitute the Gly specific A-domain
with the upstream recombination point B into the second module of PvdD
SEQ ID NO: 148 is the translation of SEQ ID NO: 147 having the residues substituted into
PvdD underlined
SEQ ID NO: 149 is the nucleotide sequence used to substitute the Gly specific A-domain
with the upstream recombination point C into the second module of PvdD
SEQ ID NO: 150 is the translation of SEQ ID NO: 149 having the residues substituted into
PvdD underlined
SEQ ID NO: 151 is the nucleotide sequence used to substitute the Gly specific A-domain
with the upstream recombination point D into the second module of PvdD
SEQ ID NO: 152 is the translation of SEQ ID NO: 151 having the residues substituted into
PvdD underlined
SEQ ID NO: 153 is the nucleotide sequence used to substitute the C- and A-domains of a
Phe specifying module into the second module of PvdD
SEQ ID NO: 154 is the translation of SEQ ID NO: 153 having the residues substituted into
PvdD underlined
SEQ ID NO: 155 is the nucleotide sequence used to substitute the Phe specific A-domain
with the upstream recombination point A into the second module of PvdD
SEQ ID NO: 156 is the translation of SEQ ID NO: 155 having the residues substituted into
PvdD underlined
SEQ ID NO: 157 is the nucleotide sequence used to substitute the Phe specific A-domain
with the upstream recombination point X into the second module of PvdD
SEQ ID NO: 158 is the translation of SEQ ID NO: 157 having the residues substituted into
PvdD underlined
SEQ ID NO: 159 is the nucleotide sequence used to substitute the Phe specific A-domain
with the upstream recombination point B into the second module of PvdD
SEQ ID NO: 160 is the translation of SEQ ID NO: 159 having the residues substituted into
PvdD underlined
SEQ ID NO: 161 is the nucleotide sequence used to substitute the Phe specific A-domain
with the upstream recombination point C into the second module of PvdD
SEQ ID NO: 162 is the translation of SEQ ID NO: 161 having the residues substituted into
PvdD underlined
SEQ ID NO: 163 is the nucleotide sequence used to substitute the Phe specific A-domain
with the upstream recombination point D into the second module of PvdD
SEQ ID NO: 164 is the translation of SEQ ID NO: 163 having the residues substituted into
PvdD underlined
SEQ ID NO: 165 is the nucleotide sequence used to substitute the A-domain from an Ala
specifying module with the upstream recombination point X into the second module of
PvdD
SEQ ID NO: 166 is the translation of SEQ ID NO: 165 having the residues substituted into
PvdD underlined
SEQ ID NO: 167 is the nucleotide sequence used to the A-domain from an Ala specifying
module with the upstream recombination point B into the second module of PvdD
SEQ ID NO: 168 is the translation of SEQ ID NO: 167 having the residues substituted into
PvdD underlined
SEQ ID NO: 169 is the nucleotide sequence used to substitute the A-domain from an Ala
specifying module with the upstream recombination point D into the second module of
PvdD
SEQ ID NO: 170 is the translation of SEQ ID NO: 169 having the residues substituted into
PvdD underlined
SEQ ID NO: 171 is the nucleotide sequence used to substitute the A-domain from an Glu
specifying module with the upstream recombination point X into the second module of
PvdD
SEQ ID NO: 172 is the translation of SEQ ID NO: 171 having the residues substituted into
PvdD underlined
SEQ ID NO: 173 is the nucleotide sequence used to the A-domain from an Glu specifying
module with the upstream recombination point B into the second module of PvdD
SEQ ID NO: 174 is the translation of SEQ ID NO: 173 having the residues substituted into
PvdD underlined
SEQ ID NO: 175 is the nucleotide sequence used to substitute the A-domain from an Glu
specifying module with the upstream recombination point D into the second module of
PvdD
SEQ ID NO: 176 is the translation of SEQ ID NO: 175 having the residues substituted into
PvdD underlined
SEQ ID NO: 177 is the nucleotide sequence used to substitute the A-domain from an Arg
specifying module, named Arg1, with the upstream recombination point X into the second
module of PvdD
SEQ ID NO: 178 is the translation of SEQ ID NO: 177 having the residues substituted into
PvdD underlined
SEQ ID NO: 179 is the nucleotide sequence used to the A-domain from an Arg specifying
module, named Arg1, with the upstream recombination point B into the second module of
PvdD
SEQ ID NO: 180 is the translation of SEQ ID NO: 179 having the residues substituted into
PvdD underlined
SEQ ID NO: 181 is the nucleotide sequence used to substitute the A-domain from an Arg
specifying module, named Arg1, with the upstream recombination point D into the second
module of PvdD
SEQ ID NO: 182 is the translation of SEQ ID NO: 181 having the residues substituted into
PvdD underlined
SEQ ID NO: 183 is the nucleotide sequence used to substitute the A-domain from an Arg
specifying module, named Arg2, with the upstream recombination point X into the second
module of PvdD
SEQ ID NO: 184 is the translation of SEQ ID NO: 183 having the residues substituted into
PvdD underlined
SEQ ID NO: 185 is the nucleotide sequence used to the A-domain from an Arg specifying
module, named Arg2, with the upstream recombination point B into the second module of
PvdD
SEQ ID NO: 186 is the translation of SEQ ID NO: 185 having the residues substituted into
PvdD underlined
SEQ ID NO: 187 is the nucleotide sequence used to substitute the A-domain from an Arg
specifying module, named Arg2, with the upstream recombination point D into the second
module of PvdD
SEQ ID NO: 188 is the translation of SEQ ID NO: 187 having the residues substituted into
PvdD underlined
SEQ ID NO: 189 is the nucleotide sequence used to substitute the Ser specific A-domain
with the upstream recombination point B and downstream recombination point DI into the
second module of PvdD
SEQ ID NO: 190 is the translation of SEQ ID NO: 189 having the residues substituted into
PvdD underlined
SEQ ID NO: 191 is the nucleotide sequence used to substitute the Ser specific A-domain
with the upstream recombination point B and downstream recombination point D2 into the
second module of PvdD
SEQ ID NO: 192 is the translation of SEQ ID NO: 191 having the residues substituted into
PvdD underlined
SEQ ID NO: 193 is the nucleotide sequence used to substitute the Ser specific A-domain
with the upstream recombination point B and downstream recombination point D3 into the
second module of PvdD
SEQ ID NO: 194 is the translation of SEQ ID NO: 193 having the residues substituted into
PvdD underlined
SEQ ID NO: 195 is the nucleotide sequence used to substitute the Ser specific A-domain
with the upstream recombination point B and downstream recombination point D4 into the
second module of PvdD
SEQ ID NO: 196 is the translation of SEQ ID NO: 195 having the residues substituted into
PvdD underlined
SEQ ID NO: 197 is the nucleotide sequence used to substitute the Ser specific A-domain
with the upstream recombination point B and downstream recombination point D5 into the
second module of PvdD
SEQ ID NO: 198 is the translation of SEQ ID NO: 197 having the residues substituted into
PvdD underlined
SEQ ID NO: 199 is the nucleotide sequence used to substitute the Ser specific A-domain
with the upstream recombination point B and downstream recombination point D6 into the
second module of PvdD
SEQ ID NO: 200 is the translation of SEQ ID NO: 199 having the residues substituted into
PvdD underlined
SEQ ID NO: 201 is the nucleotide sequence used to substitute the Ser specific A-domain
with the upstream recombination point B and downstream recombination point D7 into the
second module of PvdD
SEQ ID NO: 202 is the translation of SEQ ID NO: 201 having the residues substituted into
PvdD underlined
SEQ ID NO: 203 is the nucleotide sequence used to substitute the fhorn specific A-domain
with the upstream recombination point B and downstream recombination point DI into the
second module of PvdD
SEQ ID NO: 204 is the translation of SEQ ID NO: 203 having the residues substituted into
PvdD underlined
SEQ ID NO: 205 is the nucleotide sequence used to substitute the fhorn specific A-domain
with the upstream recombination point B and downstream recombination point D2 into the
second module of PvdD
SEQ ID NO: 206 is the translation of SEQ ID NO: 205 having the residues substituted into
PvdD underlined
SEQ ID NO: 207 is the nucleotide sequence used to substitute the fhorn specific A-domain
with the upstream recombination point B and downstream recombination point D3 into the
second module of PvdD
SEQ ID NO: 208 is the translation of SEQ ID NO: 207 having the residues substituted into
PvdD underlined
SEQ ID NO: 209 is the nucleotide sequence used to substitute the fhorn specific A-domain
with the upstream recombination point B and downstream recombination point D4 into the
second module of PvdD
SEQ ID NO: 210 is the translation of SEQ ID NO: 209 having the residues substituted into
PvdD underlined
SEQ ID NO: 211 is the nucleotide sequence used to substitute the fhorn specific A-domain
with the upstream recombination point B and downstream recombination point D5 into the
second module of PvdD
SEQ ID NO: 212 is the translation of SEQ ID NO: 211 having the residues substituted into
PvdD underlined
SEQ ID NO: 213 is the nucleotide sequence used to substitute the fhorn specific A-domain
with the upstream recombination point B and downstream recombination point D6 into the
second module of PvdD
SEQ ID NO: 214 is the translation of SEQ ID NO: 213 having the residues substituted into
PvdD underlined
SEQ ID NO: 215 is the nucleotide sequence used to substitute the fhorn specific A-domain
with the upstream recombination point B and downstream recombination point D7 into the
second module of PvdD
SEQ ID NO: 216 is the translation of SEQ ID NO: 215 having the residues substituted into
PvdD underlined
SEQ ID NO: 217 is the nucleotide sequence used to substitute the Gly specific A-domain
with the upstream recombination point B and downstream recombination point DI into the
second module of PvdD
SEQ ID NO: 218 is the translation of SEQ ID NO: 217 having the residues substituted into
PvdD underlined
SEQ ID NO: 219 is the nucleotide sequence used to substitute the Gly specific A-domain
with the upstream recombination point B and downstream recombination point D2 into the
second module of PvdD
SEQ ID NO: 220 is the translation of SEQ ID NO: 219 having the residues substituted into
PvdD underlined
SEQ ID NO: 221 is the nucleotide sequence used to substitute the Gly specific A-domain
with the upstream recombination point B and downstream recombination point D3 into the
second module of PvdD
SEQ ID NO: 222 is the translation of SEQ ID NO: 221 having the residues substituted into
PvdD underlined
SEQ ID NO: 223 is the nucleotide sequence used to substitute the Gly specific A-domain
with the upstream recombination point B and downstream recombination point D4 into the
second module of PvdD
SEQ ID NO: 224 is the translation of SEQ ID NO: 223 having the residues substituted into
PvdD underlined
SEQ ID NO: 225 is the nucleotide sequence used to substitute the Gly specific A-domain
with the upstream recombination point B and downstream recombination point D5 into the
second module of PvdD
SEQ ID NO: 226 is the translation of SEQ ID NO: 225 having the residues substituted into
PvdD underlined
SEQ ID NO: 227 is the nucleotide sequence used to substitute the Gly specific A-domain
with the upstream recombination point B and downstream recombination point D6 into the
second module of PvdD
SEQ ID NO: 228 is the translation of SEQ ID NO: 227 having the residues substituted into
PvdD underlined
SEQ ID NO: 229 is the nucleotide sequence used to substitute the Gly specific A-domain
with the upstream recombination point B and downstream recombination point D7 into the
second module of PvdD
SEQ ID NO: 230 is the translation of SEQ ID NO: 229 having the residues substituted into
PvdD underlined
SEQ ID NO: 231 is the nucleotide sequence used to substitute the A-domain from
NZ_CP020028.1.cluster004_Leu with the upstream recombination point B into pET28:ProC-TTe
SEQ ID NO: 232 is the translation of SEQ ID NO: 231 having the residues substituted into
pET28:ProC-TTe underlined
SEQ ID NO: 233 is the nucleotide sequence used to substitute the A-domain from
NZ_CP020028.1.cluster004_Leu with the upstream recombination point D into pET28:ProC-TTe
SEQ ID NO: 234 is the translation of SEQ ID NO: 233 having the residues substituted into
pET28:ProC-TTe underlined
SEQ ID NO: 235 is the nucleotide sequence used to substitute the A-domain from
the sixth module of TycC with the upstream recombination point B into pET28:ProC-TTe
SEQ ID NO: 236 is the translation of SEQ ID NO: 235 having the residues substituted into
pET28:ProC-TTe underlined
SEQ ID NO: 237 is the nucleotide sequence used to substitute the A-domain from
the sixth module of TycC with the upstream recombination point D into pET28:ProC-TTe
SEQ ID NO: 238 is the translation of SEQ ID NO: 237 having the residues substituted into
pET28:ProC-TTe underlined
SEQ ID NO: 239 is the nucleotide sequence used to substitute the A-domain from
the SrfAC with the upstream recombination point B into pET28:ProC-TTe
SEQ ID NO: 240 is the translation of SEQ ID NO: 239 having the residues substituted into
pET28:ProC-TTe underlined
SEQ ID NO: 241 is the nucleotide sequence used to substitute the A-domain from
the SrfAC with the upstream recombination point D into pET28:ProC-TTe
SEQ ID NO: 242 is the translation of SEQ ID NO: 241 having the residues substituted into
pET28:ProC-TTe underlined
> SEQ ID NO: 9 JC
SRFARLPIPQTRQEMDNLPLSYAQERQWFLWQLEPESSAYHIPTALRLRGRLDIASLQRSFAALVERHESLRTRI
ARMGDEWVQVVSADVSLALEVEVQRGLDEQRLLERVEAEIARPFDLEQGPLLRVTLLEVDADEHVLVMVQHHIVS
DGWSMQLMVEELVQLYAAYSQGLDVVLPALPIQYADYALWQRSWMEAGEKERQLAYWTGLLGGEQPVIELPLDHP
RQPLRSYRGAQLDLELEPHLALALKQLVQRKGVTMFMLLLASFQALLHRYSGQADIRVGVPIANRNRVETERLIG
FFVNTQVLKADINGRMGFDELLAQARQRALEAQAHQDLPFEQLVEALQPERSLGHNPLFQVMFNHQADSRSANQG
VQLPGLSLERMEWRSSSVAFDLTLDVHEAEDGIWASFGYATDLFEASTVERLARHWQNLLRGIVAEPGRPVAELP
LLLDEERDCLSRAWAENADEGGLPPLVQLQIQEQARLRPQAQALALE
> SEQ ID NO: 11 DC
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNPAQRECAVQGTLQQRFEEQARQRPQAVALILE
> SEQ ID NO: 13 LTT
SRFARLPIPQTRQEMDNLPLSYAQERQWFLWQLEPESSAYHIPTALRLRGRLDIASLQRSFAALVERHESLRTRI
ARMGDEWVQVVSADVSLALEVEVQRGLDEQRLLERVEAEIARPFDLEQGPLLRVTLLEVDADEHVLVMVQHHIVS
DGWSMQLMVEELVQLYAAYSQGLDVVLPALPIQYADYALWQRSWMEAGEKERQLAYWTGLLGGEQPVLELPFDRP
RPARQSHRGAQLGFELSRELVEAVRALAQREGASSFMLLLASFQALLYRYSGQADIRVGVPIANRNRVETERLIG
FFVNTQVLKADLDGRMGFDELLAQARQRALEAQAHQDLPFEQLVEALQPERNASHNPLFQVLFNHQSEIRSVTPE
VQLEDLRLEGLAWDGQTAQFDLTLDIQEDENGIWASFDYATDLFDASTVERLAGHWRNLLRGIVANPRQRLGELP
LLDAPERRQTLSEWNPAQRECAVQGTLQQRFEEQARQRPQAVALILE
> SEQ ID NO: 15 TLT
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVIELPLDHPRQ
PLRSYRGAQLDLELEPHLALALKQLVQRKGVTMFMLLLASFQALLHRYSGQADIRVGVPIANRNRVETERLIGFF
VNTQVLKADINGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNPAQRECAVQGTLQQRFEEQARQRPQAVALILE
> SEQ ID NO: 17 TTL
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERSLGHNPLFQVMFNHQADSRSANQGVQ
LPGLSLERMEWRSSSVAFDLTLDVHEAEDGIWASFGYATDLFEASTVERLARHWQNLLRGIVAEPGRPVAELPLL
LDEERDCLSRAWAENADEGGLPPLVQLQIQEQARLRPQAQALALE
> SEQ ID NO: 19 TLL
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVIELPLDHPRQ
PLRSYRGAQLDLELEPHLALALKQLVQRKGVTMFMLLLASFQALLHRYSGQADIRVGVPIANRNRVETERLIGFF
VNTQVLKADINGRMGFDELLAQARQRALEAQAHQDLPFEQLVEALQPERSLGHNPLFQVMFNHQADSRSANQGVQ
LPGLSLERMEWRSSSVAFDLTLDVHEAEDGIWASFGYATDLFEASTVERLARHWQNLLRGIVAEPGRPVAELPLL
LDEERDCLSRAWAENADEGGLPPLVQLQIQEQARLRPQAQALALE
> SEQ ID NO: 21 LTL
SRFARLPIPQTRQEMDNLPLSYAQERQWFLWQLEPESSAYHIPTALRLRGRLDIASLQRSFAALVERHESLRTRI
ARMGDEWVQVVSADVSLALEVEVQRGLDEQRLLERVEAEIARPFDLEQGPLLRVTLLEVDADEHVLVMVQHHIVS
DGWSMQLMVEELVQLYAAYSQGLDVVLPALPIQYADYALWQRSWMEAGEKERQLAYWTGLLGGEQPVLELPFDRP
RPARQSHRGAQLGFELSRELVEAVRALAQREGASSFMLLLASFQALLYRYSGQADIRVGVPIANRNRVETERLIG
FFVNTQVLKADLDGRMGFDELLAQARQRALEAQAHQDLPFEQLVEALQPERSLGHNPLFQVMFNHQADSRSANQG
VQLPGLSLERMEWRSSSVAFDLTLDVHEAEDGIWASFGYATDLFEASTVERLARHWQNLLRGIVAEPGRPVAELP
LLLDEERDCLSRAWAENADEGGLPPLVQLQIQEQARLRPQAQALALE
> SEQ ID NO: 23 LLT
SRFARLPIPQTRQEMDNLPLSYAQERQWFLWQLEPESSAYHIPTALRLRGRLDIASLQRSFAALVERHESLRTRI
ARMGDEWVQVVSADVSLALEVEVQRGLDEQRLLERVEAEIARPFDLEQGPLLRVTLLEVDADEHVLVMVQHHIVS
DGWSMQLMVEELVQLYAAYSQGLDVVLPALPIQYADYALWQRSWMEAGEKERQLAYWTGLLGGEQPVIELPLDHP
RQPLRSYRGAQLDLELEPHLALALKQLVQRKGVTMFMLLLASFQALLHRYSGQADIRVGVPIANRNRVETERLIG
FFVNTQVLKADINGRMGFDELLAQARQRALEAQAHQDLPFEQLVEALQPERNASHNPLFQVLFNHQSEIRSVTPE
VQLEDLRLEGLAWDGQTAQFDLTLDIQEDENGIWASFDYATDLFDASTVERLAGHWRNLLRGIVANPRQRLGELP
LLDAPERRQTLSEWNPAQRECAVQGTLQQRFEEQARQRPQAVALILE
> SEQ ID NO: 25 DC region 3 with 6 mutations
LVEALQPERNASHNPLFQVMFNHQSEIRSVTPEVQLEDLRLEGLAWDGQTAQFDLTLDVHEAEDGIWASFGYATD
LFDASTVERLAGHWRNLLRGIVANPRQRLGELPLLDAPERRQTLSEWNPAQRECAVQGTLQQRFEEQARQRPQAV
ALILE
> SEQ ID NO: 27 DC region 3 with 12 mutations
LVEALQPERSLGHNPLFQVMFNHQSEIRSVTPEVQLEDLRLEGLAWDGQTAQFDLTLDVHEAEDGIWASFGYATD
LFEASTVERLARHWQNLLRGIVANPRQRLGELPLLDAPERRQTLSEWNPAQRECAVQGTLQQRFEEQARQRPQAV
ALILE
> SEQ ID NO: 29 DC region 3 with a loop mutation
LVEALQPERNASHNPLFQVLFNHQADSRSANQGVQLPGLSLERMEWRSSSVAFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILE
> SEQ ID NO: 31 DC region 3 with 6 mutations and a loop mutation
LVEALQPERNASHNPLFQVMFNHQADSRSANQGVQLPGLSLERMEWRSSSVAFDLTLDVHEAEDGIWASFGYATD
LFDASTVERLAGHWRNLLRGIVANPRQRLGELPLLDAPERRQTLSEWNPAQRECAVQGTLQQRFEEQARQRPQAV
ALILE
> SEQ ID NO: 33 DC region 3 with 6 mutations and mutations to
the edges of the loop
LVEALQPERNASHNPLFQVMFNHQADSRSVTPEVQLEDLRLEGLAWRSSSVAFDLTLDVHEAEDGIWASFGYATD
LFDASTVERLAGHWRNLLRGIVANPRQRLGELPLLDAPERRQTLSEWNPAQRECAVQGTLQQRFEEQARQRPQAV
ALILE
> SEQ ID NO: 35 DC region 3 with 12 mutations and a loop mutation
LVEALQPERSLGHNPLFQVMFNHQADSRSANQGVQLPGLSLERMEWRSSSVAFDLTLDVHEAEDGIWASFGYATD
LFEASTVERLARHWQNLLRGIVANPRQRLGELPLLDAPERRQTLSEWNPAQRECAVQGTLQQRFEEQARQRPQAV
ALILE
> SEQ ID NO: 37 DC region 3 with the C-A linker from a lysine
C-A domain
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAEPGRPVAELPLLLDEERDCLSRAWAENADEGGLPPLVQLQIQEQARLRPQAQ
ALALE
> SEQ ID NO: 39 C-A domains from a Lys specific module
FARLPIPQTRQEMDNLPLSYAQERQWFLWQLEPESSAYHIPTALRLRGRLDIASLQRSFAALVERHESLRTRIAR
MGDEWVQVVSADVSLALEVEVQRGLDEQRLLERVEAEIARPFDLEQGPLLRVTLLEVDADEHVLVMVQHHIVSDG
WSMQLMVEELVQLYAAYSQGLDVVLPALPIQYADYALWQRSWMEAGEKERQLAYWTGLLGGEQPVIELPLDHPRQ
PLRSYRGAQLDLELEPHLALALKQLVQRKGVTMFMLLLASFQALLHRYSGQADIRVGVPIANRNRVETERLIGFF
VNTQVLKADINGRMGFDELLAQARQRALEAQAHQDLPFEQLVEALQPERSLGHNPLFQVMFNHQADSRSANQGVQ
LPGLSLERMEWRSSSVAFDLTLDVHEAEDGIWASFGYATDLFEASTVERLARHWQNLLRGIVAEPGRPVAELPLL
LDEERDCLSRAWAENADEGGLPPLVQLQIQEQARLRPQAQALALEGQALSYAELNARANRLAHCLIARGVGPDVL
VGIAVERSLDMVVGLLAILKAGGAYVPLDPTYPQDRLRHMLEDSAVGLLLSQEHLLPGLPLHEGLEVLSIDRLER
DASVSTDDPVVNLRPENLAYVIYTSGSTGKPKGVAISHAALAQFSRIASGYSALTPEDRILQFATLSFDGFVEQL
YPALTRGACVVLRGGDLWDTGELYRQIVEQGVTLADLPTAYWNLFLLDALAEPRRSYGALRQIHIGGEAMPLEGP
KLWRQAGMGRVRLLNTYGPTEATVVSSVFDCSAENARVGNASPIGQALPGRTLLVLDEHLGLLPVGPVGELYIAS
RAGLARAYHDRPGLTAERFLPDPFGEPGSRLYRTGDLARRRGDGVIEYMGRADHQVKIRGFRIELGEVEARLLDL
EGIREAAALALDGQLVAYLVAEGGEDETRQPALRERIRTALRASLPDYMVPSHLLFLERMPLSPNGKLDRRALPK
PD
> SEQ ID NO: 41 C-A domains from a Ser specific module
GQGNAAPRFIKADRSQPLGLSYAQQRQWFLWQLDPESTAYTIPAALRLSGSLDIAALEHSFSALIARHETLRTTF
RQQGEQAVQIIHAPRALTLMVESVPAGQTLEACVQQEMQRPFDLEKGPLLRVRLLNLATDEHVLILIQHHIVSDG
WSMPIMVDELVRLYEGYSQGREVVLTALDMQYADYALWQRNWMDAGEQARQLDYWKQQLGEQQPILELPADHPRP
VVQSHAGARLAVELAPALIDDLKQVARQQGVTLFMLLLASFQTLLHRHSGQPDIRVGVPIANRTRAETEGLIGFF
VNTQVLRAEFDLHTTFSELLQQVKQAALQAQAHQELPFEQLVEALQPQRSLSHSPLFQVMFNHQSQASAEVRALP
GLQVEALTSESYPAQFDLTLNTAEHDGGLSAGLTYATALFERSTIERMAGHWLALLQAICANAGQRIAEVPMLDA
AEQQQIVRDWNATAADFPGEHCLHSLIEAQVQATPDAPALIFAAEQLSYAQLNARANQLAHRLREAGVGPDVLVG
ICVERSVDMVIGLLAIIKAGGAYVPLDPDYPEDRLAYMMQDSGIGLLLTQSALLQGLPVQVQSLCLDQEGDWLDG
YSTANPINLSHPQNLAYVIYTSGSTGKPKGAGNSHRALVNRLHWMQKAYALDGSDTVLQKTPFSFDVSVWEFFWP
LLTGARLAVALPGDHRDPERLVQTIREHQVTTLHFVPSMLQAFLTHPQVEGCNSLRRVVCSGEALPSELAGQVLK
RLPQTGLFNLYGPTEAAIDVTHWTCTKDDVLSVPIGRPIDNLKTHILDDGLLPAAQGVSAELYLGGIGLARGYHN
RAALTAERFVPDPFDEQGGRLYRTGDLARYRDEGVIEYAGRIDHQVKIRGLRIELGEIEASLLEHGSVQEAVVID
VDGPSGKQLAAYLVAEHSGDNLRDALKVYLKETLPDYMVPTHFVWLASMPLSANGKLDRKALPTPD
> SEQ ID NO: 43 C-A domains from a fhOrn specific module
QAPGAPTAPALLPVGRDQPLPLSYAQERQWFLWQLEPQSAAYHIPSALRLKGQLDLGALQRSFDTLLARHESLRT
HLRQERDRTVQIISPQLSLQIAHAEVQEAQLKARVEAEIAQPFNLEQGPLLRVSLLRIAADEHVLVLVQHHIVSD
GWSMQLMVEELVQLYAAYSQGQVLQWPALPIQYADYAVWQRNWMEAGEKARQLAYWRDMLGGEQSVLALPFDHPR
PAVQSHRGARLAFELPGALTQGLKALAKQQDVTLFMLLLASFQTLLHRYSGQEEIRVGVPIANRNRSETERLIGF
FVNTQVLKADLHGQMSVEQLLQQARQRALDAQAHQDLPFEQLVEALQPERSLSHNPLFQVMFNHQTDVGQAQVQQ
QLPNLSVEGLEWESKTAHFDLDLDIQESTEGIWATLGYAQDLFEASTVQRMARHWQNLLQGMVADPRQNLSQLNL
LDNAERQQILQLWNQSDAGFSAKRLVHELVADRAGETPEAVAVKFGEQQLTYGELDRQANRLAHALIARGVGPEV
RVAIAMPRSAEIMVAFLAVMKAGGVYVPLDIEYPRDRLLYMMQDSRARLLLTHSAVQQRLPIPDGLEALAVDQAQ
AWSADDDTAPEVALDGDNLAYVIYTSGSTGMPKGVAVSHGPLVAHIIATGERYETSPADCELHFMSFAFDGSHEG
WMHPLINGASVLIRDDSLWLPEYTYQQMHRHHVTMAVFPPVYLQQLAEHAERDGNPPKVRVYCFGGDAVAQASYD
LAWRALKPTYLFNGYGPTETVVTPLLWKARRGDPCGAVYAPIGTLLGNRSGYVLDAQLNLQPIGVAGELYLGGEG
VARGYLERPALTAERFVPDPFGKPGSRVYRSGDLTRGRPDGVVDYLGRVDHQVKIRGFRIELGEIEARLREQDSV
GETVVVAQEGPSGKQLVAYVVPLDPLLVDDAVAQSTCREALRRALKTRLPDYMVPTHLMFLERMPLTPNGKLDRK
GLPRPD
> SEQ ID NO: 45 Linker plus A-domain from a Lys specific module
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAEPGRPVAELPLLLDEERDCLSRAWAENADEGGLPPLVQLQIQEQARLRPQAQ
ALALEGQALSYAELNARANRLAHCLIARGVGPDVLVGIAVERSLDMVVGLLAILKAGGAYVPLDPTYPQDRLRHM
LEDSAVGLLLSQEHLLPGLPLHEGLEVLSIDRLERDASVSTDDPVVNLRPENLAYVIYTSGSTGKPKGVAISHAA
LAQFSRIASGYSALTPEDRILQFATLSFDGFVEQLYPALTRGACVVLRGGDLWDTGELYRQIVEQGVTLADLPTA
YWNLFLLDALAEPRRSYGALRQIHIGGEAMPLEGPKLWRQAGMGRVRLLNTYGPTEATVVSSVFDCSAENARVGN
ASPIGQALPGRTLLVLDEHLGLLPVGPVGELYIASRAGLARAYHDRPGLTAERFLPDPFGEPGSRLYRTGDLARR
RGDGVIEYMGRADHQVKIRGFRIELGEVEARLLDLEGIREAAALALDGQLVAYLVAEGGEDETRQPALRERIRTA
LRASLPDYMVPSHLLFLERMPLSPNGKLDRRALPKPD
> SEQ ID NO: 47 Ser_X
LVEALQPERNASHNPLFQVLENHQSEIRSVTPEVQLEDLRLEGLAWDGQTAQFDLTLDIQEDENGIWASFDYATD
LFDASTVERLAGHWRNLLRGIVANAGQRIAEVPMLDAAEQQQIVRDWNATAADFPGEHCLHSLIEAQVQATPDAP
ALIFAAEQLSYAQLNARANQLAHRLREAGVGPDVLVGICVERSVDMVIGLLAIIKAGGAYVPLDPDYPEDRLAYM
MQDSGIGLLLTQSALLQGLPVQVQSLCLDQEGDWLDGYSTANPINLSHPQNLAYVIYTSGSTGKPKGAGNSHRAL
VNRLHWMQKAYALDGSDTVLQKTPFSFDVSVWEFFWPLLTGARLAVALPGDHRDPERLVQTIREHQVTTLHFVPS
MLQAFLTHPQVEGCNSLRRVVCSGEALPSELAGQVLKRLPQTGLFNLYGPTEAAIDVTHWTCTKDDVLSVPIGRP
IDNLKTHILDDGLLPAAQGVSAELYLGGIGLARGYHNRAALTAERFVPDPFDEQGGRLYRTGDLARYRDEGVIEY
AGRIDHQVKIRGLRIELGEIEASLLEHGSVQEAVVIDVDGPSGKQLAAYLVAEHSGDNLRDALKVYLKETLPDYM
VPTHFVWLASMPLSANGKLDRKALPTPD
> SEQ ID NO: 49 fhOrn_X
LVEALQPERNASHNPLFQVLFNHQSEIRSVTPEVQLEDLRLEGLAWDGQTAQFDLTLDIQEDENGIWASFDYATD
LEDASTVERLAGHWRNLLRGIVADPRQNLSQLNLLDNAERQQILQLWNQSDAGFSAKRLVHELVADRAGETPEAV
AVKFGEQQLTYGELDRQANRLAHALIARGVGPEVRVAIAMPRSAEIMVAFLAVMKAGGVYVPLDIEYPRDRLLYM
MQDSRARLLLTHSAVQQRLPIPDGLEALAVDQAQAWSADDDTAPEVALDGDNLAYVIYTSGSTGMPKGVAVSHGP
LVAHIIATGERYETSPADCELHFMSFAFDGSHEGWMHPLINGASVLIRDDSLWLPEYTYQQMHRHHVTMAVFPPV
YLQQLAEHAERDGNPPKVRVYCFGGDAVAQASYDLAWRALKPTYLFNGYGPTETVVTPLLWKARRGDPCGAVYAP
IGTLLGNRSGYVLDAQLNLQPIGVAGELYLGGEGVARGYLERPALTAERFVPDPFGKPGSRVYRSGDLTRGRPDG
VVDYLGRVDHQVKIRGFRIELGEIEARLREQDSVGETVVVAQEGPSGKQLVAYVVPLDPLLVDDAVAQSTCREAL
RRALKTRLPDYMVPTHLMFLERMPLTPNGKLDRKGLPRPD
> SEQ ID NO: 51 l_CP008696.1.cluster009_A1
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAQPGQRLGDLPLLAASEQNKLLHEWAPASVEFPSEHGVHQRVEAQARKNPEAE
ALLFAGQSLNYQALNARANRLAHKLIELGVGPEVRVGVAMQRTPEMVVALLAVLKAGGAYVPLDPDYPQDRLAHM
LRDSQAQILLTESALLSLLPAVESLQTLQLDAQPGWLDGYSPDNPAPRATADNLAYVIYTSGSTGLPKGVAIAHR
NVLALIDWSSRVYSADDLQGVLASTSICFDLSVWELFVTLSSGGFIVLARNALELPELVDRDRVRLINTVPSAIA
ALQRSGQIPPGVRIINLAGEPLKQALVDSLYQQPGLQHVYDLYGPSEDTTYSTYTRREAGGQANIGRAISNTQSY
ILSPDLQPVPVGSAGELYLAGAGVTRGYLARPGLTAEKFVPNPFSSDGGRVYRTGDLTRYRADGVIEYIGRIDHQ
VKVRGFRIELGEIEARLVQQAAVREAFVLAQDGDNGQQLVAYIVPSETTEAIEAQAALRENIKAALKAHLPDYMV
PTYLLFLEALPLTPNGKLDRKALPKVD
> SEQ ID NO: 53 2_CP006852.1.cluster006_A4
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVADTQQRIGQLPLLDDQEQQAVIHDWNATARDYPSQRCVHQLIEAQVARTPDAP
ALVFGQQRLSYAQLNRRANRLAHRLIAAGVGPDVLVGLALERSIEMVVGLLAVLKAGGAYVPLDPEYPRERLAYM
LEDSGVKLLLTQAHLLQQLPIPQGLDHLVLGESWFEGYSDSNPGIVLDGENLAYVIYTSGSTGQPKGAGNRHSAL
TNRLQWMQEAYGLGASDTVLQKTPFSFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTHWTCVDEGHDTVPIGEP
IANLRTHVLDADLSPVPVGVAGELYLGGAGLARSYHRRPGLTAERFVPCPFHPGARLYRSGDRVRQRADGVIEYL
GRLDHQVKLRGLRIELGEIEARLLEHPAVREASVQVVDGKQLVAYVVLQPNGDDWRERLSTHLASHLPDYMVPAQ
WVVLEHMPLSPNGKLDRKALPKPE
> SEQ ID NO: 55 3_CP011507.1.cluster002_A1
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVADCDGRVGELPLLDDAQWQQRVHAWNNTHQAFLPDVGVHRLLEAQAQARPDAT
ALVONGQALSYAQLNRRANRLAHRLRAAGVGPDVLVAVALDRSVDMVLALLATLKAGGAYVPLDPQFPADRLAFM
LEDSRARVLLTAGDLHQRLPVAADQQVLFISEQEDARHSSDNPHVELSGEHLAYVIYTSGSTGKPKGVMVRHGAL
SSFTQGMADTLSIDADARLLSLTTFSFDIFALELYVPLSVGATVVLADKEVSLDPEAILSLLHDQAINVVQATPS
TWRMLLDSERRAVLHGVKCLCGGEALPADLAQRMLAQQGTVWNLYGPTETTIWSAAHPLVEPLPFVGRPIANTSL
FILNAELTLSPVGTSGELLIGGVGLARGYHGRAAMTAERFVPNPFARNGERLYRTGDLARYRVDGVVEYIGRVDH
QVKVRGFRIELGEIEACLREQSDVREAVVVAENDQLLAYLVTHTATSEADQGALREALKAALRDVLPDYMVPAHM
LFLARLPLTPNGKLDRKALPKPD
> SEQ ID NO: 57 4_CP003041.1.cluster006_A2
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQQLSQLSLLDATEQQQILQLWNRTESGFSAERLVHELVGDRARETPDAV
AVKFDAQTLTYGELDRQANRLAHALIARGVGPEVRVAIAMPRSAEIMVAFLAVMKAGGVYVPLDIEYPRDRLLYM
MQDSRSKMLLTHSAVQHRLPIPDGLDVLAVDQVQAWSDYSDTAPTVALDGDNLAYVIYTSGSTGLPKGVAVSHGP
LVAHIIATGERYETSPADCELHFMSFAFDGSHEGWMHPLINGASVLIRDDSLWLPEYTYEQMHRHNVTMAVFPPV
YLQQLAEHAERDGNPPAVRVYCFGGDAVAQASYDLAWRALKPKYLFNGYGPTETVVTPLLWKARKGDPCGAVYAP
IGTLLGNRSGYVLDAQLNLQPIGVAGELYLGGEGVARGYLERPALTAEREVPDPFGKPGSRVYRSGDLTRGRPDG
VVDYLGRVDHQVKIRGFRIELGEIEARLREQASVGETVVVAQEGPTGKQLVAYVVPLDRTLLDDAVAQSTGRETL
RRALKTRLPDYMVPTHLMFLERMPLTPNGKLDRKGLPLPD
> SEQ ID NO: 59 5_AP013068.1.cluster003_A1
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPELAVGELPLLDAEELRQQLQGWNATARDYGCETVHRLFEAQVASQPDAPA
LAFGTEHLSYAQLNARANRLAHKLRSLGVGPESLVGVACERSVELVVGLLAVLKAGGAYVPFDPEYPRDRLAYLE
DDSAIRLLLTQSHLLGELPLPEGVTSLCLDQDSDALEAFSGANPEVPLAPNNLAYVIYTSGSTGKPKGAGNTHGA
LHNRLAWMQEAYALDAGDSVLQKTPFSFDVSVWEFFWPLMVGARLAVAAPGDHRDPSRLLALIEAHRVTTLHFVP
SMLQAFVSQLALEEQGARQCASLKRIVCSGEALPAELQGQVFAELPGVGLFNLYGPTEAAIDVTHWTCREEGRDS
VPIGQPIANLATHILDARLNPVPVGVAGELYLAGAGLARGYHRRAGLSAERFVANPFAPGERMYRTGDLARYRTD
GVIEYLGRIDHQVKIRGFRIELGEIEARLQSHAGVREAVVVAVDGASGKQLVAYLVAAEAGAEEGALRESIKAHL
GATLPDYMVPAQFVLLTAMPLSPNGKLDRKALPKPD
> SEQ ID NO: 61 6_CP010945.1.cluster006_A1
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAQPKTAIGDLQLLAPSEHDQQENWSAAPCTPASQWLPELLGEQARLTPERTAL
MWDGGSLDFAELHAQANRLAHYLRDKGVGPDVCVAIAAERSPQLLIGLLAIIKAGGAYVPLDPDYPAERLAYMLR
DSGVELLLTQTQLLDRLPATDGVSVIAMDALHLENWPSQAPGLHLHGDNLAYVIYTSGSTGQPKGVGNTHTALAE
RLQWMQNTYRLNDTDVLMQKAPISFDVSVWECFWPLITGARLLIAGPGEHRDPHRIAQLVQQYGVTTLHFVPPLL
SLFIDEPLTAECTSLRRVFSGGEALPAELRNRVLEQLPAVQLHNRYGPTETAINVTHWQCSAADGERSPIGRPLG
NVICRVLDSDLNPVPAGVPGELCISGIGLARGYLGRPGLTAERFVVDPLSEQGARLYRTGDRARWTAEGVIEYLG
RLDQQVKVRGFRVEPEEIEARLLAQNGVAQAVVLVRETAAGAQLIGYYTATANSEAEDTQTARLKTALAVELPEY
MVPAQLMRLGEMPLSPSGKLDRRALPEPR
> SEQ ID NO: 63 7_AM181176.4.cluster005_A2
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAEPQRPVAALPLLMPAEWQRTVVEWNRTEMDVPQHLTFAQLFEQQAERTPQRD
ALCFEGQRLSYAELNRRANQVAHYLRAQGVVANCPVALCVERSLELLIGLLGILKAGGAYVPLDPGYPAERLAYM
LEDAQPALFLGQQGLLEQLGGDLPRLRLDADAALLAAQPESNPAALAGPDDLAYIMYTSGSTGKPKGTLVTHTSV
VNLAWARIHGLYRRYTDQPMRTSFNYSFAFDSSVAELILLLDGHSLYLTPEDVRYDPAALAQFFQETRLDAFECT
PAQLKSLLETDGVRRGETYLPRFVLFGGDAVDAQLWQRLPSISGSRFFNTYGPTECTVDATGCAVDDFPQRPIIG
RPIANVRTYVLDAFLNPMPVGVPGELHIGGAGVTLGYLNRAEQTAKVFINDPFSPLPQARMYKSGDLVRWLPDGQ
LEYLGRMDHQVKIRGFRVELGEIEALIGAQPGVRQAVVLAREDVQGDKRLVAYVTCDQPADMNAWRNRLGAALPD
YMVPSAFVVLDELPLTDNGKLNRKALPAPD
> SEQ ID NO: 65 8_CP000680.1.cluster003_A3
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAMPQASLADLPLLSESEVAEVEAWNAPIPVPNSIQLLPERIAEQARLRPTAIA
LVHGQQRLSFAELEARANCLAHQLIARGVGAEVRVGVALERGIELFVALLAVLKAGGAYVPLDPDYPGERLRYML
EDAGVKLLLSHQAALPRLPEVAGIEVLDLDHLPLNDQPEQAPEVNIHHEQLAYLIYTSGSTGKPKGVAVAHGAIA
MHCQAIGERYELTAEDRELHFLSVSFDGAHERWLTPLSHGARVVIRDQQLWSVQQTYDCLIEEGISVVALPPSYL
RQLAEWAEQCGKAPGVKTYCFAGEAFSRELLQQVIRSLQPQWIINGYGPTETVVTPTTWRVPAATADFDTAYAPI
GDRVGARQGYVLDADLNLLPVGVAGELYLGGLLARGYLDRPGATAERFVPNPYRPGERLYRTGDRVRLGADGQLE
YLGRLDQQIKLRGFRIEIGEVEAALKACAGVGESLVVVKDSAAGKRLVGYVSGQALSESELKAQLKQRLPSHMVP
SHILALERLPLLPNGKLDRQSLPEPQ
> SEQ ID NO: 67 9_CP011972.1.cluster002_A4
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVAAPDAPLFSLGALTVELPGEVREPAPQVLQLWDRQVESQPDALAARCLDRTLN
TRALDQAANQLAHHLIGRGVCESQPVAVLMERSLDWLTAVLAIFKAGGVYMPLDIKAPDARLQQMLSNAKAKVLL
CAEGDVRQTSLDVAGCEGLAWTPALWQDLPVSRPDITLSADSAAYVIHTSGSTGQPKGVVVSQGALASYVHGVLE
QLQLAPEASMALVSTIAADLGHTVLFGALCSGRTLHVLTESLGFDPDAFAAYMAEHQVGVLKIVPGHLAALLQAA
QPADVLPQHALIVGGEACSPALVEQVRQLKPGCRVINHYGPSETTVGVLTHEVPALSELNAIPCGSELVREEAGT
GLQKAEALLPPSRASSLPQEPAKVPVGKPLPGASAYVLDDVLNPVATQVAGELYIGGDSVARGYIGQPALTAERF
VPDPFAQDGSRVYRSGDRMRRNHQGLLEFIGRADDQVKVRGYRVEPAEVARVLLSLPSVAQVSVLALPVDEDESR
LQLVAYCVAATGASLTIDSLREQLTARLPDYMVPAQILLLDQLPLTANGKLDKRALPKPG
> SEQ ID NO: 69 Partial A-domain Ser 1
LVEALQPERNASHNPLFQVLENHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFGASDTVLQKTPFSFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTHWTCVDEGHDTVPIGEP
IPDLSWYILDRDLNPVPRGAVGELYIGRAGLARGYLRRPGLSATRFVPNPFPGGAGERLYRTGDLARFQADGNIE
YIGRIDHQVKVRGFRIELGEIEAALAGLAGVRDAVVLAHDGVGGTQLVGYVVADSAEDAERLRESLRESLKRHLP
DYMVPAHLMLLERMPLTVNGKLDRQALPQPD
> SEQ ID NO: 71 Partial A-domain Lys 1
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFTPEDRILQFATLSFDGFVEQLYPALTRGACVVLRGGDLWDTGELYRQIVEQGVTLADLPTAY
WNLFLLDALAEPRRSYGALRQIHIGGEAMPLEGPKLWRQAGMGRVRLLNTYGPTEATVVSSVFDCSAENARVGNA
SPIGQALPDLSWYILDRDLNPVPRGAVGELYIGRAGLARGYLRRPGLSATRFVPNPFPGGAGERLYRTGDLARFQ
ADGNIEYIGRIDHQVKVRGFRIELGEIEAALAGLAGVRDAVVLAHDGVGGTQLVGYVVADSAEDAERLRESLRES
LKRHLPDYMVPAHLMLLERMPLTVNGKLDRQALPQPD
> SEQ ID NO: 73 Partial A-domain Ser 2
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHSAL
TNRLQWMQEAYGLGASDTVLQKTPFSFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGITETTVHVTYRPVSEADLKGGLVSPI
GGTIPDLSWYILDRDLNPVPRGAVGELYIGRAGLARGYLRRPGLSATRFVPNPFPGGAGERLYRTGDLARFQADG
NIEYIGRIDHQVKVRGFRIELGEIEAALAGLAGVRDAVVLAHDGVGGTQLVGYVVADSAEDAERLRESLRESLKR
HLPDYMVPAHLMLLERMPLTVNGKLDRQALPQPD
> SEQ ID NO: 75 Partial A-domain Lys 2
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHAAL
AQFSRIASGYSALTPEDRILQFATLSFDGFVEQLYPALTRGACVVLRGGDLWDTGELYRQIVEQGVTLADLPTAY
WNLFLLDALAEPRRSYGALRQIHIGGEAMPLEGPKLWRQAGMGRVRLLNTYGITETTVHVTYRPVSEADLKGGLV
SPIGGTIPDLSWYILDRDLNPVPRGAVGELYIGRAGLARGYLRRPGLSATRFVPNPFPGGAGERLYRTGDLARFQ
ADGNIEYIGRIDHQVKVRGFRIELGEIEAALAGLAGVRDAVVLAHDGVGGTQLVGYVVADSAEDAERLRESLRES
LKRHLPDYMVPAHLMLLERMPLTVNGKLDRQALPQPD
> SEQ ID NO: 77 Partial A-domain Ser 3
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFDERDVWTLFHSYAFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTYRPVSEADLKGGLVSPI
GGTIPDLSWYILDRDLNPVPRGAVGELYIGRAGLARGYLRRPGLSATRFVPNPFPGGAGERLYRTGDLARFQADG
NIEYIGRIDHQVKVRGFRIELGEIEAALAGLAGVRDAVVLAHDGVGGTQLVGYVVADSAEDAERLRESLRESLKR
HLPDYMVPAHLMLLERMPLTVNGKLDRQALPQPD
> SEQ ID NO: 79 Partial A-domain Lys 3
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFDERDVWTLFHSYAFDGFVEQLYPALTRGACVVLRGGDLWDTGELYRQIVEQGVTLADLPTAY
WNLFLLDALAEPRRSYGALRQIHIGGEAMPLEGPKLWRQAGMGRVRLLNTYGPTEATVVSTYRPVSEADLKGGLV
SPIGGTIPDLSWYILDRDLNPVPRGAVGELYIGRAGLARGYLRRPGLSATRFVPNPFPGGAGERLYRTGDLARFQ
ADGNIEYIGRIDHQVKVRGFRIELGEIEAALAGLAGVRDAVVLAHDGVGGTQLVGYVVADSAEDAERLRESLRES
LKRHLPDYMVPAHLMLLERMPLTVNGKLDRQALPQPD
> SEQ ID NO: 81 Partial A-domain Ser 4
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYSDSNPGIVLDGENLAYVIYTSGSTGQPKGAGNRHSAL
TNRLQWMQEAYGLGASDTVLQKTPFSFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTHWTCVDEGHDTVPIGEP
IANLRTHVLDADLSPVPVGVAGELYLGGAGLARSYHRRPGLTAERFVPCPFHPGARLYRSGDRVRQRADGVIEYL
GRLDHQVKLRGLRIELGEIEARLLEHPAVREASVQVVDGKQLVAYVVLQPNGDDWRERLSTHLASHLPDYMVPAQ
WVVLEHMPLSPNGKLDRKALPKPE
> SEQ ID NO: 83 Partial A-domain Lys 4
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGSVSTDDPVVNLRPENLAYVIYTSGSTGKPKGVAISHAA
LAQFSRIASGYSALTPEDRILQFATLSFDGFVEQLYPALTRGACVVLRGGDLWDTGELYRQIVEQGVTLADLPTA
YWNLFLLDALAEPRRSYGALRQIHIGGEAMPLEGPKLWRQAGMGRVRLLNTYGPTEATVVSSVFDCSAENARVGN
ASPIGQALPGRTLLVLDEHLGLLPVGPVGELYIASRAGLARAYHDRPGLTAERFLPDPFGEPGSRLYRTGDLARR
RGDGVIEYMGRADHQVKIRGFRIELGEVEARLLDLEGIREAAALALDGQLVAYLVAEGGEDETRQPALRERIRTA
LRASLPDYMVPSHLLFLERMPLSPNGKLDRRALPKPD
> SEQ ID NO: 85 Partial A-domain Ser 5
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGQPKGAGNRHSAL
TNRLQWMQEAYGLGASDTVLQKTPFSFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTHWTCVDEGHDTVPIGEP
IANLRTHVLDADLSPVPVGVAGELYLGGAGLARSYHRRPGLTAERFVPCPFHPGARLYRSGDRVRQRADGVIEYL
GRLDHQVKLRGLRIELGEIEARLLEHPAVREASVQVVDGKQLVAYVVLQPNGDDWRERLSTHLASHLPDYMVPAQ
WVVLEHMPLSPNGKLDRKALPKPE
> SEQ ID NO: 87 Partial A-domain Lys 5
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGVAISHAAL
AQFSRIASGYSALTPEDRILQFATLSFDGFVEQLYPALTRGACVVLRGGDLWDTGELYRQIVEQGVTLADLPTAY
WNLFLLDALAEPRRSYGALRQIHIGGEAMPLEGPKLWRQAGMGRVRLLNTYGPTEATVVSSVFDCSAENARVGNA
SPIGQALPGRTLLVLDEHLGLLPVGPVGELYIASRAGLARAYHDRPGLTAERFLPDPFGEPGSRLYRTGDLARRR
GDGVIEYMGRADHQVKIRGFRIELGEVEARLLDLEGIREAAALALDGQLVAYLVAEGGEDETRQPALRERIRTAL
RASLPDYMVPSHLLFLERMPLSPNGKLDRRALPKPD
> SEQ ID NO: 89 Partial A-domain Ser 6
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFDERDVWTLFHSYAFDVSVWEFFWPLMSGARLVVAAPGDHRDPARLISVITAEQVTTVHFVPS
MLQAFLQDAAVTRCQSLQRIVCSGEALPVDAQQQVFAKLPQAGLYNLYGPTEAAIDVTHWTCVDEGHDTVPIGEP
IANLRTHVLDADLSPVPVGVAGELYLGGAGLARSYHRRPGLTAERFVPCPFHPGARLYRSGDRVRQRADGVIEYL
GRLDHQVKLRGLRIELGEIEARLLEHPAVREASVQVVDGKQLVAYVVLQPNGDDWRERLSTHLASHLPDYMVPAQ
WVVLEHMPLSPNGKLDRKALPKPE
> SEQ ID NO: 91 Partial A-domain Lys 6
LVEALQPERNASHNPLFQVLENHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNPAQRECAVQGTLQQRFEEQARQRPQAV
ALILDEQRLSYGELNARANRLAHCLIARGVGADVPVGLALERSLDMLVGLLAILKAGGAYLPLDPAAPEERLAHI
LDDSGVRLLLTQGHLLERLPRQAGVEVLAIDGLVLDGYAESDPLPTLSADNLAYVIYTSGSTGKPKGTLLTHRNA
LRLFSATEAWFGFDERDVWTLFHSYAFDGFVEQLYPALTRGACVVLRGGDLWDTGELYRQIVEQGVTLADLPTAY
WNLFLLDALAEPRRSYGALRQIHIGGEAMPLEGPKLWRQAGMGRVRLLNTYGPTEATVVSSVFDCSAENARVGNA
SPIGQALPGRTLLVLDEHLGLLPVGPVGELYIASRAGLARAYHDRPGLTAERFLPDPFGEPGSRLYRTGDLARRR
GDGVIEYMGRADHQVKIRGFRIELGEVEARLLDLEGIREAAALALDGQLVAYLVAEGGEDETRQPALRERIRTAL
RASLPDYMVPSHLLFLERMPLSPNGKLDRRALPKPD
> SEQ ID NO: 93 Ser_A
LVEALQPERNASHNPLFQVLFNHQSEIRSVTPEVQLEDLRLEGLAWDGQTAQFDLTLDIQEDENGIWASFDYATD
LFDASTVERLAGHWLALLQAICANAGQRIAEVPMLDAAEQQQIVRDWNATAADFPGEHCLHSLIEAQVQATPDAP
ALIFAAEQLSYAQLNARANQLAHRLREAGVGPDVLVGICVERSVDMVIGLLAIIKAGGAYVPLDPDYPEDRLAYM
MQDSGIGLLLTQSALLQGLPVQVQSLCLDQEGDWLDGYSTANPINLSHPQNLAYVIYTSGSTGKPKGAGNSHRAL
VNRLHWMQKAYALDGSDTVLQKTPFSFDVSVWEFFWPLLTGARLAVALPGDHRDPERLVQTIREHQVTTLHFVPS
MLQAFLTHPQVEGCNSLRRVVCSGEALPSELAGQVLKRLPQTGLFNLYGPTEAAIDVTHWTCTKDDVLSVPIGRP
IDNLKTHILDDGLLPAAQGVSAELYLGGIGLARGYHNRAALTAERFVPDPFDEQGGRLYRTGDLARYRDEGVIEY
AGRIDHQVKIRGLRIELGEIEASLLEHGSVQEAVVIDVDGPSGKQLAAYLVAEHSGDNLRDALKVYLKETLPDYM
VPTHFVWLASMPLSANGKLDRKALPTPD
> SEQ ID NO: 95 Ser_B
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAAEQQQIVRDWNATAADFPGEHCLHSLIEAQVQATPDAP
ALIFAAEQLSYAQLNARANQLAHRLREAGVGPDVLVGICVERSVDMVIGLLAIIKAGGAYVPLDPDYPEDRLAYM
MQDSGIGLLLTQSALLQGLPVQVQSLCLDQEGDWLDGYSTANPINLSHPQNLAYVIYTSGSTGKPKGAGNSHRAL
VNRLHWMQKAYALDGSDTVLQKTPFSFDVSVWEFFWPLLTGARLAVALPGDHRDPERLVQTIREHQVTTLHFVPS
MLQAFLTHPQVEGCNSLRRVVCSGEALPSELAGQVLKRLPQTGLFNLYGPTEAAIDVTHWTCTKDDVLSVPIGRP
IDNLKTHILDDGLLPAAQGVSAELYLGGIGLARGYHNRAALTAERFVPDPFDEQGGRLYRTGDLARYRDEGVIEY
AGRIDHQVKIRGLRIELGEIEASLLEHGSVQEAVVIDVDGPSGKQLAAYLVAEHSGDNLRDALKVYLKETLPDYM
VPTHFVWLASMPLSANGKLDRKALPTPD
> SEQ ID NO: 97 Ser C
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPEQQQIVRDWNATAADFPGEHCLHSLIEAQVQATPDAP
ALIFAAEQLSYAQLNARANQLAHRLREAGVGPDVLVGICVERSVDMVIGLLAIIKAGGAYVPLDPDYPEDRLAYM
MQDSGIGLLLTQSALLQGLPVQVQSLCLDQEGDWLDGYSTANPINLSHPQNLAYVIYTSGSTGKPKGAGNSHRAL
VNRLHWMQKAYALDGSDTVLQKTPFSFDVSVWEFFWPLLTGARLAVALPGDHRDPERLVQTIREHQVTTLHFVPS
MLQAFLTHPQVEGCNSLRRVVCSGEALPSELAGQVLKRLPQTGLFNLYGPTEAAIDVTHWTCTKDDVLSVPIGRP
IDNLKTHILDDGLLPAAQGVSAELYLGGIGLARGYHNRAALTAERFVPDPFDEQGGRLYRTGDLARYRDEGVIEY
AGRIDHQVKIRGLRIELGEIEASLLEHGSVQEAVVIDVDGPSGKQLAAYLVAEHSGDNLRDALKVYLKETLPDYM
VPTHFVWLASMPLSANGKLDRKALPTPD
> SEQ ID NO: 99 Ser_D
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNATAADFPGEHCLHSLIEAQVQATPDAP
ALIFAAEQLSYAQLNARANQLAHRLREAGVGPDVLVGICVERSVDMVIGLLAIIKAGGAYVPLDPDYPEDRLAYM
MQDSGIGLLLTQSALLQGLPVQVQSLCLDQEGDWLDGYSTANPINLSHPQNLAYVIYTSGSTGKPKGAGNSHRAL
VNRLHWMQKAYALDGSDTVLQKTPFSFDVSVWEFFWPLLTGARLAVALPGDHRDPERLVQTIREHQVTTLHFVPS
MLQAFLTHPQVEGCNSLRRVVCSGEALPSELAGQVLKRLPQTGLFNLYGPTEAAIDVTHWTCTKDDVLSVPIGRP
IDNLKTHILDDGLLPAAQGVSAELYLGGIGLARGYHNRAALTAERFVPDPFDEQGGRLYRTGDLARYRDEGVIEY
AGRIDHQVKIRGLRIELGEIEASLLEHGSVQEAVVIDVDGPSGKQLAAYLVAEHSGDNLRDALKVYLKETLPDYM
VPTHFVWLASMPLSANGKLDRKALPTPD
> SEQ ID NO: 101 fhOrn_A
LVEALQPERNASHNPLFQVLFNHQSEIRSVTPEVQLEDLRLEGLAWDGQTAQFDLTLDIQEDENGIWASFDYATD
LFDASTVERLAGHWQNLLQGMVADPRQNLSQLNLLDNAERQQILQLWNQSDAGFSAKRLVHELVADRAGETPEAV
AVKFGEQQLTYGELDRQANRLAHALIARGVGPEVRVAIAMPRSAEIMVAFLAVMKAGGVYVPLDIEYPRDRLLYM
MQDSRARLLLTHSAVQQRLPIPDGLEALAVDQAQAWSADDDTAPEVALDGDNLAYVIYTSGSTGMPKGVAVSHGP
LVAHIIATGERYETSPADCELHFMSFAFDGSHEGWMHPLINGASVLIRDDSLWLPEYTYQQMHRHHVTMAVFPPV
YLQQLAEHAERDGNPPKVRVYCFGGDAVAQASYDLAWRALKPTYLFNGYGPTETVVTPLLWKARRGDPCGAVYAP
IGTLLGNRSGYVLDAQLNLQPIGVAGELYLGGEGVARGYLERPALTAEREVPDPFGKPGSRVYRSGDLTRGRPDG
VVDYLGRVDHQVKIRGFRIELGEIEARLREQDSVGETVVVAQEGPSGKQLVAYVVPLDPLLVDDAVAQSTCREAL
RRALKTRLPDYMVPTHLMFLERMPLTPNGKLDRKGLPRPD
> SEQ ID NO: 103 fhOrn_B
LVEALQPERNASHNPLFQVLFNHQSEIRSVTPEVQLEDLRLEGLAWDGQTAQFDLTLDIQEDENGIWASFDYATD
LEDASTVERLAGHWRNLLRGIVANPRQRLGELPLLDNAERQQILQLWNQSDAGFSAKRLVHELVADRAGETPEAV
AVKFGEQQLTYGELDRQANRLAHALIARGVGPEVRVAIAMPRSAEIMVAFLAVMKAGGVYVPLDIEYPRDRLLYM
MQDSRARLLLTHSAVQQRLPIPDGLEALAVDQAQAWSADDDTAPEVALDGDNLAYVIYTSGSTGMPKGVAVSHGP
LVAHIIATGERYETSPADCELHFMSFAFDGSHEGWMHPLINGASVLIRDDSLWLPEYTYQQMHRHHVTMAVFPPV
YLQQLAEHAERDGNPPKVRVYCFGGDAVAQASYDLAWRALKPTYLFNGYGPTETVVTPLLWKARRGDPCGAVYAP
IGTLLGNRSGYVLDAQLNLQPIGVAGELYLGGEGVARGYLERPALTAERFVPDPFGKPGSRVYRSGDLTRGRPDG
VVDYLGRVDHQVKIRGFRIELGEIEARLREQDSVGETVVVAQEGPSGKQLVAYVVPLDPLLVDDAVAQSTCREAL
RRALKTRLPDYMVPTHLMFLERMPLTPNGKLDRKGLPRPD
> SEQ ID NO: 105 fhOrn_C
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERQQILQLWNQSDAGFSAKRLVHELVADRAGETPEAV
AVKFGEQQLTYGELDRQANRLAHALIARGVGPEVRVAIAMPRSAEIMVAFLAVMKAGGVYVPLDIEYPRDRLLYM
MQDSRARLLLTHSAVQQRLPIPDGLEALAVDQAQAWSADDDTAPEVALDGDNLAYVIYTSGSTGMPKGVAVSHGP
LVAHIIATGERYETSPADCELHFMSFAFDGSHEGWMHPLINGASVLIRDDSLWLPEYTYQQMHRHHVTMAVFPPV
YLQQLAEHAERDGNPPKVRVYCFGGDAVAQASYDLAWRALKPTYLFNGYGPTETVVTPLLWKARRGDPCGAVYAP
IGTLLGNRSGYVLDAQLNLQPIGVAGELYLGGEGVARGYLERPALTAERFVPDPFGKPGSRVYRSGDLTRGRPDG
VVDYLGRVDHQVKIRGFRIELGEIEARLREQDSVGETVVVAQEGPSGKQLVAYVVPLDPLLVDDAVAQSTCREAL
RRALKTRLPDYMVPTHLMFLERMPLTPNGKLDRKGLPRPD
> SEQ ID NO: 107 fhOrn_D
LVEALQPERNASHNPLFQVLFNHQSEIRSVTPEVQLEDLRLEGLAWDGQTAQFDLTLDIQEDENGIWASFDYATD
LFDASTVERLAGHWRNLLRGIVANPRQRLGELPLLDAPERRQTLSEWNQSDAGFSAKRLVHELVADRAGETPEAV
AVKFGEQQLTYGELDRQANRLAHALIARGVGPEVRVAIAMPRSAEIMVAFLAVMKAGGVYVPLDIEYPRDRLLYM
MQDSRARLLLTHSAVQQRLPIPDGLEALAVDQAQAWSADDDTAPEVALDGDNLAYVIYTSGSTGMPKGVAVSHGP
LVAHIIATGERYETSPADCELHFMSFAFDGSHEGWMHPLINGASVLIRDDSLWLPEYTYQQMHRHHVTMAVFPPV
YLQQLAEHAERDGNPPKVRVYCFGGDAVAQASYDLAWRALKPTYLFNGYGPTETVVTPLLWKARRGDPCGAVYAP
IGTLLGNRSGYVLDAQLNLQPIGVAGELYLGGEGVARGYLERPALTAERFVPDPFGKPGSRVYRSGDLTRGRPDG
VVDYLGRVDHQVKIRGFRIELGEIEARLREQDSVGETVVVAQEGPSGKQLVAYVVPLDPLLVDDAVAQSTCREAL
RRALKTRLPDYMVPTHLMFLERMPLTPNGKLDRKGLPRPD
> SEQ ID NO: 110 1_TycB1_Pro
LAGHLQQIADCVANNSGVELCQIPLLTEAETSQLLAKRTETAADYPAATMHELFSRQAEKTPEQVAVVFADQHLT
YRELDEKSNQLARFLRKKGIGTGSLVGTLLDRSLDMIVGILGVLKAGGAFVPIDPELPAERIAYMLTHSRVPLVV
TQNHLRAKVTTPTETIDINTAVIGEESRAPIESLNQPHDLFYIIYTSGTTGQPKGVMLEHRNMANLMHFTFDQTN
IAFHEKVLQYTTCSFDVCYQEIFSTLLSGGQLYLITNELRRHVEKLFAFIQEKQISILSLPVSFLKFIFNEQDYA
QSFPRCVKHIITAGEQLVVTHELQKYLRQHRVFLHNHYGPSETHVVTTCTMDPGQAIPELPPIGKPISNTGIYIL
DEGLQLKPEGIVGELYISGANVGRGYLHQPELTAEKFLDNPYQPGERMYRTGDLALWLPDGQLEFLGRIDHQVKI
RGHRIELGEIESRLLNHPAIKEAVVIDRADETGGKFLCAYVVLQKALSDEEMRAYLAQALPEYMIPSFFVTLERI
PVTPNGKTDRRALPKPE
> SEQ ID NO: 112 2_TycC6_Leu
LAGHLQQIADCVANNPHIRLGEIDMLLPEEKQQILAGFNDTAVSYALDKTLHQLFEEQVDKTPDQAALLFSEQSL
TYSELNERANRLARVLRAKGVGPDRLVAIMAERSPEMVIGILGILKAGGAYVPVDPGYPQERIQYLLEDSNAALL
LSQAHLLPLLAQVSSELPECLDLNAELDAGLSGSNLPAVNQPTDLAYVIYTSGTTGKPKGVMIPHQGIVNCLQWR
RDEYGFGPSDKALQVFSFAFDGFVASLFAPLLGGATCVLPQEAAAKDPVALKKLMAATEVTHYYGVPSLFQAILD
CSTTTDFNQLRCVTLGGEKLPVQLVQKTKEKHPAIEINNEYGPTENSVVTTISRSIEAGQAITIGRPLANVQVYI
VDEQHHLQPIGVVGELCIGGAGLARGYLNKPELTAEKFVANPFRPGERMYKTGDLVKWRTDGTIEYIGRADEQVK
VRGYRIEIGEIESAVLAYQGIDQAVVVARDDDATAGSYLCAYFVAATAVSVSGLRSHLAKELPAYMIPSYFVELD
QLPLSANGKVDRKALPKPQ
> SEQ ID NO: 114 3_SrfAC_Leu
LAGHLQQIADCVANNPDQPVSTINLVDDREREFLLTGLNPPAQAHETKPLTYWFKEAVNANPDAPALTYSGQTLS
YRELDEEANRIARRLQKHGAGKGSVVALYTKRSLELVIGILGVLKAGAAYLPVDPKLPEDRISYMLADSAAACLL
THQEMKEQAAELPYTGTTLFIDDQTRFEEQASDPATAIDPNDPAYIMYTSGTTGKPKGNITTHANIQGLVKHVDY
MAFSDQDTFLSVSNYAFDAFTFDFYASMLNAARLIIADEHTLLDTERLTDLILQENVNVMFATTALFNLLTDAGE
DWMKGLRCILFGGERASVPHVRKALRIMGPGKLINCYGPTEGTVFATAHVVHDLPDSISSLPIGKPISNASVYIL
NEQSQLQPFGAVGELCISGMGVSKGYVNRADLTKEKFIENPFKPGETLYRTGDLARWLPDGTIEYAGRIDDQVKI
RGHRIELEEIEKQLQEYPGVKDAVVVADRHESGDASINAYLVNRTQLSAEDVKAHLKKQLPAYMVPQTFTFLDEL
PLTTNGKVNKRLLPKPD
> SEQ ID NO: 116 4_NZ_CP021920.1.cluster002_Phe
LAGHLQQIADCVANHPDIQLSQIEIMSEDERNMLFYQFNDTKTDYPKDKTICQLFAERAARIPDHTALVFEDQKL
TYRELDERSNQLAGFLREKGVEPNTAVGIMVERSPEMIIGILGILKAGAAYLPLDPAYPEDRIKYILEDSQTKIL
LTQDALMKERTLIKDAAIMKIDIRDNQIVRRNADRLPHFPHAGDLAYIIYTSGSTGKPKGVLIEQKGLCNLVHAV
IDLMQLKTDSRVIQFASLSFDASAFEIFSALAAGAALVLGRQEDMMPGQALTSFLRHHEITHATLPPTVLNVLDE
SQLDHLKVIVSAGSACSEELATRWSGKRMFINAYGPTETTVCATAGVYRGTGRPHIGSPIANTNIYIMDQNVQPV
ATGIVGEVCVGGISLARGYLNKPELTAEKFIPHPFVPGERLYRTGDLARWLPDGNLEFLGRIDHQVKIRGYRIEL
GEIENQLLKHDNIEEAAVIARTGKDNNDYLCAYIVSQKQLTATEVSEWLEKELPHYMIPAYVVKLDKLPLTSNDK
VDRKALPEPD
> SEQ ID NO: 118 5_NZ_CP020028.1.cluster004_Leu
LAGHLQQIADCVANCPKMRIEDIEIVPEEEGSLLLHDFNRTEAEYPKDQTINALFEERAEQRADHPALVWGEQTL
TYRELNEQANRLAKVLRARGVKADDIVAIMTERSMEMVIGILGTLKAGGAYLPIDPNYPEERIHYMLEDSGASML
LTQKHLRDKLTYHGPIMDVDGEDLKHLELDGHANLQPANKPEDLAYIIYTSGSTGKPKGVMVEHRGIINLWHFFQ
EQWGVNGSDRMLQFASSSFDASVWEMFTILLGGGTLYLVSRDIINNLNEFARFVNENQITIALLPPTYLAGIEPE
KLPALKKLVTGGSAITKELVTRWKDSVEYMNAYGPSESSVIATAWTYREEDMGYSSVPIGKPIANTRIYIMDEHQ
KLLPLGAAGEMCVAGDGLARGYLHRPELTAEKFVVNPYEAGEKLYRTGDLVRWLPDGNIEFLGRIDDQVKIRGFR
IELGEIEAQLQKHPLVQEVAVIAREDKQKEKYLAAYITAEGEPEAEELREQLLQELPDYMVPSSFMQLEHMPMTP
SGKIDRKALPEPE
> SEQ ID NO: 120 6_NZ_CM000756.1.cluster012_Leu
LAGHLQQIADCVANNLSISVNKIDMIPQEEKRFLLYEHNDTNVDFSKNQLIHKLFEEQVERTPDSIAVVFEDKQL
TYRELNEKSNQLARALRENGVGSDKIVGILLERSVDVIVGIMGILKAGGAYLPIDPTYPIDRIKYILQDSQTEIL
LTQDKLINLVDCTEVDDISIINIHNEHLFKYGTENLRIDSSSKDLVYVIYTSGSTGKPKGVMVEHHSLVNLCNWH
NSFNKISETDKNASYASISFDAFAWEVFPYIIAGSEIHIINDNLKLDITKLNKYFIEKEISISFLPTQVCEQFLM
LENTSLRRLLTGGDKLNYFENKSYQIVNNYGPTENSVVTTSFIIENSYDNIPIGKPICNTKVFILNESNGLCPLG
VPGELCISGEGLARGYLNRPELTAEKFIPNPFIPGERMYRTGDLVRMLPDGNIEFLGRIDHQVKIRGFRIELGEI
ESQLLKHKEVKEAVVIAREDNNNHQYLCAYFTSETSKGETRVQEIRKFLTKELPEYMIPAFFVQLDKLPLTTNGK
VDRKALPSPD
> SEQ ID NO: 142 -Gly CA
SRTTDAVSTIPLADRQQPLALSFAQGRMWFLDQLEPLSSLYNIPQGIRLFGAVEVENLRLALEQIVARHESLRTT
FKILDGHSVQVVAPPAGFALPVIEVGGSDGSEREAEALRVVEEESQRPFDLSKGPLLRALLLRLDRDEHVLLLTL
HHIISDGWSLGVLFAELGALYEAFCKGEEAHLPELPIQYGDYAAWQREWLSGEVLERQTAYWREQLGGMAPSLNL
PTDRPRPAVQTSRGARQSFLVPPSLTRSLVELSRREGVTLYMTLLAAFQVLLQRYTGQDDISVGSPIAGRTTAET
EGLIGLFINTLVMRTDLSGDPTFRELLERVRQVALGAYAHQDVPFEKLVEQLQPERDMSRTPLFQVMFILQNTPG
LAPSLEGLTVEPLPIENETARFDLTLAMAESADGLPGEFEYNADLFDAETIARLLGHFTILLEGIAAGADVSISA
LPLLTGAESRRMLVEWNDTREAYAADRRVHELFEAQAARTPDASAVESQGRALSYAELNRRANQLARHLRRMGVG
PEVLVGICVEKSVEMLVGLLGILKAGGAYVPLDPAFPAERLAFMAEDAELPVLLTESRLTAVVPPNPTRRTVLLD
SDWELIAGESGDDLSDTGGGENTAYVIYTSGSTGRPKGVQVSHRALVNFLHSMRGRPGIEAGDTLLAVTTLSFDI
AGLELYLPLLTGARVVVAGREELSDGALLSERISRSGATVMQATPATWRLLLGAGWRGKSDLRIFCGGEALAREL
ANQLLEKCAELWNLYGPTETTIWSAIHRVETTEGAVSVGRPIANTRVYVLDKNLRPVPAGVPGELLIGGDGLARG
YLNRPDLTAEKFVPDPFGDAPGARLYRTGDLARYSPGGELEILNRVDTQVKIRGFRIEPGEIEGVLEQHPAIRQA
VVVARDDGEGEKRLVGYVVARPESALNVNELRRYLLGTLPEYMVPSAFVMLDELPLTPNGKIDREALPQPDAA
> SEQ ID NO: 144 -GlyA
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHFTILLEGIAAGADVSISALPLL
TGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEGVLEQHPAIRQAVVVA
RDDGEGEKRLVGYVVARPESALNVNELRRYLLGTLPEYMVPSAFVMLDELPLTPNGKIDREALPQPDAA
> SEQ ID NO: 146 -GlyX
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVAGADVSISALPLL
TGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEGVLEQHPAIRQAVVVA
RDDGEGEKRLVGYVVARPESALNVNELRRYLLGTLPEYMVPSAFVMLDELPLTPNGKIDREALPQPDAA
> SEQ ID NO: 148 -GlyB
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEGVLEQHPAIRQAVVVA
RDDGEGEKRLVGYVVARPESALNVNELRRYLLGTLPEYMVPSAFVMLDELPLTPNGKIDREALPQPDAA
> SEQ ID NO: 150 -GlyC
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEGVLEQHPAIRQAVVVA
RDDGEGEKRLVGYVVARPESALNVNELRRYLLGTLPEYMVPSAFVMLDELPLTPNGKIDREALPQPDAA
> SEQ ID NO: 152 -GlyD
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEGVLEQHPAIRQAVVVA
RDDGEGEKRLVGYVVARPESALNVNELRRYLLGTLPEYMVPSAFVMLDELPLTPNGKIDREALPQPDAA
> SEQ ID NO: 154 -PheCA
SRTTDAVSTIPLADRQQPLALSFAQGRLWFLDQLEPNSPFYNNPVAVRLKGQLDIARLEEALNALIQRHEALRTR
FVAVDGKPVAVVDAELRLKLSVEPAVDVEQQARAEALEPFDLATGPLIRARLLQVNAAEHIALVTLHHIISDGWS
TGVLVRELSALYAGRELAPLAIQYGDFAAWQREWLSGEVLEAQLNYWREQLQDAPPVLELATDRARPAVQSFRGS
HYRFQVPSEVAGELAELSRREGVTLFMVLLAAYQVLLSRYAGGQEDVVVGTPIANRQRTEVEGLIGFFVNTLVLR
TKLAGEPSVRELLGRVRETCLGAYAHQDLPFETLVETLQPERKLSHAPLFQTMLVWANAPAERLELGGLEVEAVE
AESGTARFDLTLEMGEGAGGELWGSLEYASDLWDESTVARMAGHFCELLGQMAGKVERPVTELELAVPEQAQWNA
TEAEYPRGVTIHALVEEQAARDPERAALRFEGQALSYGELVARVKETSREMGPRQLVAISLERGFAQVVAQLAAL
EAGGAYVPVDPSYPEERREYMLADSGAGVVVNGEGIVKREAGREVDGLAYMIYTSGSTGRPKGTMLRHEGLCNLA
RWQQRAFGITRESRVLQFAPSSFDASVWETFMALANGATLVLGRQEVLRQMEALHKLLVEEKITHVTLPPTVLEA
LEAEGLPDLQVVIAAGEACGRELVEKWGRGRRFFNAYGPTETTVCASAYECKVGERVAPAIGRPIANMQMWVLDE
WGRPAPVGVSGELHIGGVGLAAGYWERPELTAEKFVETAYGRVYKSGDVGRWRGDGVVEYVGRRDTQVKLRGYRI
ELGEVEEALRSCAGVRAAGVGVDGDRLVGYVVGGDIAEVRRELRGRLPEYMVPGVVMALEEMPLLPNGKIDRQAL
PQPDAA
> SEQ ID NO: 156 -PheA
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHFCELLGQMAGKVERPVTELELA
VPEQAQWNATEAEYPRGVTIHALVEEQAARDPERAALRFEGQALSYGELVARVKETSREMGPRQLVAISLERGFA
QVVAQLAALEAGGAYVPVDPSYPEERREYMLADSGAGVVVNGEGIVKREAGREVDGLAYMIYTSGSTGRPKGTML
RHEGLCNLARWQQRAFGITRESRVLQFAPSSFDASVWETFMALANGATLVLGRQEVLRQMEALHKLLVEEKITHV
TLPPTVLEALEAEGLPDLQVVIAAGEACGRELVEKWGRGRRFFNAYGPTETTVCASAYECKVGERVAPAIGRPIA
NMQMWVLDEWGRPAPVGVSGELHIGGVGLAAGYWERPELTAEKFVETAYGRVYKSGDVGRWRGDGVVEYVGRRDT
QVKLRGYRIELGEVEEALRSCAGVRAAGVGVDGDRLVGYVVGGDIAEVRRELRGRLPEYMVPGVVMALEEMPLLP
NGKIDRQALPQPDAA
> SEQ ID NO: 158 -PheX
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVAKVERPVTELELA
VPEQAQWNATEAEYPRGVTIHALVEEQAARDPERAALRFEGQALSYGELVARVKETSREMGPRQLVAISLERGFA
QVVAQLAALEAGGAYVPVDPSYPEERREYMLADSGAGVVVNGEGIVKREAGREVDGLAYMIYTSGSTGRPKGTML
RHEGLCNLARWQQRAFGITRESRVLQFAPSSFDASVWETFMALANGATLVLGRQEVLRQMEALHKLLVEEKITHV
TLPPTVLEALEAEGLPDLQVVIAAGEACGRELVEKWGRGRRFFNAYGPTETTVCASAYECKVGERVAPAIGRPIA
NMQMWVLDEWGRPAPVGVSGELHIGGVGLAAGYWERPELTAEKFVETAYGRVYKSGDVGRWRGDGVVEYVGRRDT
QVKLRGYRIELGEVEEALRSCAGVRAAGVGVDGDRLVGYVVGGDIAEVRRELRGRLPEYMVPGVVMALEEMPLLP
NGKIDRQALPQPDAA
> SEQ ID NO: 160 -PheB
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DVPEQAQWNATEAEYPRGVTIHALVEEQAARDPERAALRFEGQALSYGELVARVKETSREMGPRQLVAISLERGF
AQVVAQLAALEAGGAYVPVDPSYPEERREYMLADSGAGVVVNGEGIVKREAGREVDGLAYMIYTSGSTGRPKGTM
LRHEGLCNLARWQQRAFGITRESRVLQFAPSSFDASVWETFMALANGATLVLGRQEVLRQMEALHKLLVEEKITH
VTLPPTVLEALEAEGLPDLQVVIAAGEACGRELVEKWGRGRRFFNAYGPTETTVCASAYECKVGERVAPAIGRPI
ANMOMWVLDEWGRPAPVGVSGELHIGGVGLAAGYWERPELTAEKFVETAYGRVYKSGDVGRWRGDGVVEYVGRRD
TQVKLRGYRIELGEVEEALRSCAGVRAAGVGVDGDRLVGYVVGGDIAEVRRELRGRLPEYMVPGVVMALEEMPLL
PNGKIDRQALPQPDAA
> SEQ ID NO: 162 -PheC
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPEQAQWNATEAEYPRGVTIHALVEEQAARDPERAALRFEGQALSYGELVARVKETSREMGPRQLVAISLERGF
AQVVAQLAALEAGGAYVPVDPSYPEERREYMLADSGAGVVVNGEGIVKREAGREVDGLAYMIYTSGSTGRPKGTM
LRHEGLCNLARWQQRAFGITRESRVLQFAPSSFDASVWETFMALANGATLVLGRQEVLRQMEALHKLLVEEKITH
VTLPPTVLEALEAEGLPDLQVVIAAGEACGRELVEKWGRGRRFFNAYGPTETTVCASAYECKVGERVAPAIGRPI
ANMQMWVLDEWGRPAPVGVSGELHIGGVGLAAGYWERPELTAEKFVETAYGRVYKSGDVGRWRGDGVVEYVGRRD
TQVKLRGYRIELGEVEEALRSCAGVRAAGVGVDGDRLVGYVVGGDIAEVRRELRGRLPEYMVPGVVMALEEMPLL
PNGKIDRQALPQPDAA
> SEQ ID NO: 164 -PheD
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNATEAEYPRGVTIHALVEEQAARDPERAALRFEGQALSYGELVARVKETSREMGPRQLVAISL
ERGFAQVVAQLAALEAGGAYVPVDPSYPEERREYMLADSGAGVVVNGEGIVKREAGREVDGLAYMIYTSGSTGRP
KGTMLRHEGLCNLARWQQRAFGITRESRVLQFAPSSFDASVWETFMALANGATLVLGRQEVLRQMEALHKLLVEE
KITHVTLPPTVLEALEAEGLPDLQVVIAAGEACGRELVEKWGRGRRFFNAYGPTETTVCASAYECKVGERVAPAI
GRPIANMQMWVLDEWGRPAPVGVSGELHIGGVGLAAGYWERPELTAEKFVETAYGRVYKSGDVGRWRGDGVVEYV
GRRDTQVKLRGYRIELGEVEEALRSCAGVRAAGVGVDGDRLVGYVVGGDIAEVRRELRGRLPEYMVPGVVMALEE
MPLLPNGKIDRQALPQPDAA
> SEQ ID NO: 166 -AlaX
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVAEPETAVSRLRLL
TLSEEHQVLNEWNNTAREFDITGGIHRLFEAQVERTPTATALVVGHEWISYHELNGRANSLAHHLVQSGVKAEDR
VGILMERSPAMVVSLLAVLKAGGCYVPLDPQYPRERLEFMQADAAVSALLTTRAVATTCGLQTDHVIYVDEVEQT
ATENLNVEISSQQLAYLIYTSGSTGVPKGVAITHGNATTFIHWASEIFDEKALNGVLFSTSICFDLSIFELFVTL
SNGGKVILADNALQLPTLPAANEVTLINTVPSAMTELIRSGAVPKSVRMVNLAGEALSKDLVTEIYTTTNVETVY
NLYGPSEDTTYSTFTATSPGEPVTIGKPIANTRAYVLDEQFQIAPVGVVGELYLGGAGLARGYWQRSDLTATKFI
PDNFSPMPGGRLYRTGDLARYLDNGELEFLGRADHQVKVRGYRIELGEIESELRQHAQVREAVVVARAERLVAYT
VSTSTVNSVELREHLRQRLPEYMVPSALVQLPSMPLTPNGKLDRKALPQPDAA
> SEQ ID NO: 168 -AlaB
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDEYGIWASFDYATDLFDASTVERLAGHWRNLLRGIVANPRQRLGELPLL
DLSEEHQVLNEWNNTAREFDITGGIHRLFEAQVERTPTATALVVGHEWISYHELNGRANSLAHHLVQSGVKAEDR
VGILMERSPAMVVSLLAVLKAGGCYVPLDPQYPRERLEFMQADAAVSALLTTRAVATTCGLQTDHVIYVDEVEQT
ATENLNVEISSQQLAYLIYTSGSTGVPKGVAITHGNATTFIHWASEIFDEKALNGVLFSTSICFDLSIFELFVTL
SNGGKVILADNALQLPTLPAANEVTLINTVPSAMTELIRSGAVPKSVRMVNLAGEALSKDLVTEIYTTTNVETVY
NLYGPSEDTTYSTFTATSPGEPVTIGKPIANTRAYVLDEQFQIAPVGVVGELYLGGAGLARGYWQRSDLTATKFI
PDNFSPMPGGRLYRTGDLARYLDNGELEFLGRADHQVKVRGYRIELGEIESELRQHAQVREAVVVARAERLVAYT
VSTSTVNSVELREHLRQRLPEYMVPSALVQLPSMPLTPNGKLDRKALPQPDAA
> SEQ ID NO: 170 -AlaD
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNNTAREFDITGGIHRLFEAQVERTPTATALVVGHEWISYHELNGRANSLAHHLVQSGVKAEDR
VGILMERSPAMVVSLLAVLKAGGCYVPLDPQYPRERLEFMQADAAVSALLTTRAVATTCGLQTDHVIYVDEVEQT
ATENLNVEISSQQLAYLIYTSGSTGVPKGVAITHGNATTFIHWASEIFDEKALNGVLFSTSICFDLSIFELFVTL
SNGGKVILADNALQLPTLPAANEVTLINTVPSAMTELIRSGAVPKSVRMVNLAGEALSKDLVTEIYTTTNVETVY
NLYGPSEDTTYSTFTATSPGEPVTIGKPIANTRAYVLDEQFQIAPVGVVGELYLGGAGLARGYWQRSDLTATKFI
PDNFSPMPGGRLYRTGDLARYLDNGELEFLGRADHQVKVRGYRIELGEIESELRQHAQVREAVVVARAERLVAYT
VSTSTVNSVELREHLRQRLPEYMVPSALVQLPSMPLTPNGKLDRKALPQPDAA
> SEQ ID NO: 172 -GluX
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVADAGRKLSEVAVM
SAEEQQQLVEGLNQTSAEYPHHSCIHELFERQAAETPEAVAVVFGEEQVSYGELNERANRLAHYLREKGVKPESR
VGLLLERSVETIVGVLGILKAGGAYVPLDPEYPQERLAFMLTDSGIEVVITQAALGERLREQPQLRLLCLDTEGA
SISAYADTVLPSDATPDNLAYVIYTSGSTGNPKGVMIRHASALNLLTALRQSIYSQLTAPLRVSVNAPLSFDASV
KQLVQLLDGHTLVMVPEEARRDGAALVQYLARQRVEVLDCTPSQLRLMLGADVSTGPLGGLRAALVGGEELDERL
WRQLSEITDITGTAFFNVYGPTECTVDATVCRVSGQARRPSIGRPLANVSVYVLDRNLLPVPVGVAGQLHIGGEG
VARCYLNRPELTAEKFIPDGLGKVPGARLYRTGDLVRYLPDGQLEYLGRSDHQVKVRGYRIELGEIESALSLHPA
VREAAATVRADEAGDKRLVAYVVFDDGQTPSTGELRAYLQAHLPDYMIPHLFVTLEALPLTVNGKIDREALPQPD
AA
> SEQ ID NO: 174 -GluB
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAEEQQQLVEGLNQTSAEYPHHSCIHELFERQAAETPEAVAVVFGEEQVSYGELNERANRLAHYLREKGVKPESR
VGLLLERSVETIVGVLGILKAGGAYVPLDPEYPQERLAFMLTDSGIEVVITQAALGERLREQPQLRLLCLDTEGA
SISAYADTVLPSDATPDNLAYVIYTSGSTGNPKGVMIRHASALNLLTALRQSIYSQLTAPLRVSVNAPLSFDASV
KQLVQLLDGHTLVMVPEEARRDGAALVQYLARQRVEVLDCTPSQLRLMLGADVSTGPLGGLRAALVGGEELDERL
WRQLSEITDITGTAFFNVYGPTECTVDATVCRVSGQARRPSIGRPLANVSVYVLDRNLLPVPVGVAGQLHIGGEG
VARCYLNRPELTAEKFIPDGLGKVPGARLYRTGDLVRYLPDGQLEYLGRSDHQVKVRGYRIELGEIESALSLHPA
VREAAATVRADEAGDKRLVAYVVFDDGQTPSTGELRAYLQAHLPDYMIPHLFVTLEALPLTVNGKIDREALPQPD
AA
> SEQ ID NO: 176 -GluD
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSELNQTSAEYPHHSCIHELFERQAAETPEAVAVVFGEEQVSYGELNERANRLAHYLREKGVKPESR
VGLLLERSVETIVGVLGILKAGGAYVPLDPEYPQERLAFMLTDSGIEVVITQAALGERLREQPQLRLLCLDTEGA
SISAYADTVLPSDATPDNLAYVIYTSGSTGNPKGVMIRHASALNLLTALRQSIYSQLTAPLRVSVNAPLSFDASV
KQLVQLLDGHTLVMVPEEARRDGAALVQYLARQRVEVLDCTPSQLRLMLGADVSTGPLGGLRAALVGGEELDERL
WRQLSEITDITGTAFFNVYGPTECTVDATVCRVSGQARRPSIGRPLANVSVYVLDRNLLPVPVGVAGQLHIGGEG
VARCYLNRPELTAEKFIPDGLGKVPGARLYRTGDLVRYLPDGQLEYLGRSDHQVKVRGYRIELGEIESALSLHPA
VREAAATVRADEAGDKRLVAYVVFDDGQTPSTGELRAYLQAHLPDYMIPHLFVTLEALPLTVNGKIDREALPQPD
AA
> SEQ ID NO: 178 -Arg1X
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVADVHQPVARIDLL
ALPERNLLLQTWNDTRADYPYEQCVHQLFEEQVRKTPEAIAVVQDDIELSYAQLNVRANRLAHYLIKQGVRPDTR
VAICVERSFAMVIGLLAILKAGGTYLPLDPTHPSERLVELLDDAQPVVLLADATGRRALGTHVPNTTTTLSLDEP
LPADAQSEATTSANPVPQQLGLTSSHLAYVIYTSGSTGKPKGVMVEHRQLACQITSLRRQWQLTNADRVLQFNNI
AFDVATSEIFGALISGARLVLRTAEWLSSTTKFWALCESFGITYIDVPTQFWSRLDDDATQHLPPRLKVICIGGE
AAPSQTVRRWLERHPGRPVLANCYGPTETTVTATVGCPDRNDSHHVSIGRPIANTRIYLLDGHGQPVPLGAVGEI
YIGGAGVARGYLNRPQLTAERFLQDPFCNEPGARMYRTGDLARYRANGNIEYLGRADQQVKIRGFRIELGEIEAR
LAAHDSVREAVVIAREDGGNKRLVAYVTPRSDAAIEVSALRAHLARQLPEYMVPAAFVQIDALPLTPNGKVDRQA
LPQPDAA
> SEQ ID NO: 180 -Arg1B
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DLPERNLLLQTWNDTRADYPYEQCVHQLFEEQVRKTPEAIAVVQDDIELSYAQLNVRANRLAHYLIKQGVRPDTR
VAICVERSFAMVIGLLAILKAGGTYLPLDPTHPSERLVELLDDAQPVVLLADATGRRALGTHVPNTTTTLSLDEP
LPADAQSEATTSANPVPQQLGLTSSHLAYVIYTSGSTGKPKGVMVEHRQLACQITSLRRQWQLTNADRVLQFNNI
AFDVATSEIFGALISGARLVLRTAEWLSSTTKFWALCESFGITYIDVPTQFWSRLDDDATQHLPPRLKVICIGGE
AAPSQTVRRWLERHPGRPVLANCYGPTETTVTATVGCPDRNDSHHVSIGRPIANTRIYLLDGHGQPVPLGAVGEI
YIGGAGVARGYLNRPQLTAERFLQDPFCNEPGARMYRTGDLARYRANGNIEYLGRADQQVKIRGFRIELGEIEAR
LAAHDSVREAVVIAREDGGNKRLVAYVTPRSDAAIEVSALRAHLARQLPEYMVPAAFVQIDALPLTPNGKVDRQA
LPQPDAA
> SEQ ID NO: 182 -Arg1D
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNDTRADYPYEQCVHQLFEEQVRKTPEAIAVVQDDIELSYAQLNVRANRLAHYLIKQGVRPDTR
VAICVERSFAMVIGLLAILKAGGTYLPLDPTHPSERLVELLDDAQPVVLLADATGRRALGTHVPNTTTTLSLDEP
LPADAQSEATTSANPVPQQLGLTSSHLAYVIYTSGSTGKPKGVMVEHRQLACQITSLRRQWQLTNADRVLQFNNI
AFDVATSEIFGALISGARLVLRTAEWLSSTTKFWALCESFGITYIDVPTQFWSRLDDDATQHLPPRLKVICIGGE
AAPSQTVRRWLERHPGRPVLANCYGPTETTVTATVGCPDRNDSHHVSIGRPIANTRIYLLDGHGQPVPLGAVGEI
YIGGAGVARGYLNRPQLTAERFLQDPFCNEPGARMYRTGDLARYRANGNIEYLGRADQQVKIRGFRIELGEIEAR
LAAHDSVREAVVIAREDGGNKRLVAYVTPRSDAAIEVSALRAHLARQLPEYMVPAAFVQIDALPLTPNGKVDRQA
LPQPDAA
> SEQ ID NO: 184 -Arg2X
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPSSPIRDLVLL
DDSEHAQLVLGWNATAAPLPECTLHQLFEQQARRAPDRIAVVDGHTELTYAELNAKANRLAHHLRSLGVGPDVLV
ALCMERSADLLVGLLAVLKAGGAYLPLDPAYPAARLAYMLDDAMPAVLLTESRWLTHLSSHQVPVCCLDREWPSL
ARFPDSDPPAAAMPPNVAYVIYTSGSTGNPKGVLTTHRNVVNQLLGHARLCELSDSDRVLQFASIGFDVSVEEIE
ATLLAGATLVLRSEELLEGGAVFSEWVSRHALTVLDLPTAFWHEWVRCLDEGEAFLPPMLRLVVIGGEKARADAA
HAWLRLTQARPIRLINAYGPTETTVGVTAYELPPDFTGLDIPIGRPCPNTQLYILDTEQQPVPIGACGELYIAGA
GVARGYLRRPGLTGEKFVANPFDAGTRMYRSGDLVRYLPDGNIVYLGRIDEQVKIRGFRIEPGEIEAGLMALEGV
RQAVVVTREDSPGNRRLAAYVVAQDGAVVQAAKLRAGLQARLPEYMVPTHILLLGQLPLTPNGKMDRKALPQPDA
A
> SEQ ID NO: 186 -Arg2B
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DDSEHAQLVLGWNATAAPLPECTLHQLFEQQARRAPDRIAVVDGHTELTYAELNAKANRLAHHLRSLGVGPDVLV
ALCMERSADLLVGLLAVLKAGGAYLPLDPAYPAARLAYMLDDAMPAVLLTESRWLTHLSSHQVPVCCLDREWPSL
ARFPDSDPPAAAMPPNVAYVIYTSGSTGNPKGVLTTHRNVVNQLLGHARLCELSDSDRVLQFASIGFDVSVEEIF
ATLLAGATLVLRSEELLEGGAVFSEWVSRHALTVLDLPTAFWHEWVRCLDEGEAFLPPMLRLVVIGGEKARADAA
HAWLRLTQARPIRLINAYGPTETTVGVTAYELPPDFTGLDIPIGRPCPNTQLYILDTEQQPVPIGACGELYIAGA
GVARGYLRRPGLTGEKFVANPFDAGTRMYRSGDLVRYLPDGNIVYLGRIDEQVKIRGFRIEPGEIEAGLMALEGV
RQAVVVTREDSPGNRRLAAYVVAQDGAVVQAAKLRAGLQARLPEYMVPTHILLLGQLPLTPNGKMDRKALPQPDA
A
> SEQ ID NO: 188 -Arg2D
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAPERRQTLSEWNATAAPLPECTLHQLFEQQARRAPDRIAVVDGHTELTYAELNAKANRLAHHLRSLGVGPDVLV
ALCMERSADLLVGLLAVLKAGGAYLPLDPAYPAARLAYMLDDAMPAVLLTESRWLTHLSSHQVPVCCLDREWPSL
ARFPDSDPPAAAMPPNVAYVIYTSGSTGNPKGVLTTHRNVVNQLLGHARLCELSDSDRVLQFASIGFDVSVEEIF
ATLLAGATLVLRSEELLEGGAVFSEWVSRHALTVLDLPTAFWHEWVRCLDEGEAFLPPMLRLVVIGGEKARADAA
HAWLRLTQARPIRLINAYGPTETTVGVTAYELPPDFTGLDIPIGRPCPNTQLYILDTEQQPVPIGACGELYIAGA
GVARGYLRRPGLTGEKFVANPFDAGTRMYRSGDLVRYLPDGNIVYLGRIDEQVKIRGFRIEPGEIEAGLMALEGV
RQAVVVTREDSPGNRRLAAYVVAQDGAVVQAAKLRAGLQARLPEYMVPTHILLLGQLPLTPNGKMDRKALPQPDA
A
> SEQ ID NO: 190 -SerD1
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGGTIPDLSWYILDRDLNPVPRGAVGELYIGRAGLARGY
LRRPGLSATRFVPNPFPGGAGERLYRTGDLARFQADGNIEYIGRIDHQVKVRGFRIELGEIEAALAGLAGVRDAV
VLAHDGVGGTQLVGYVVADSAEDAERLRESLRESLKRHLPDYMVPAHLMLLERMPLTVNGKLDRQALPQPDAA
> SEQ ID NO: 192 -SerD2
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDRDLNPVPRGAVGELYIGRAGLARGY
LRRPGLSATRFVPNPFPGGAGERLYRTGDLARFQADGNIEYIGRIDHQVKVRGFRIELGEIEAALAGLAGVRDAV
VLAHDGVGGTQLVGYVVADSAEDAERLRESLRESLKRHLPDYMVPAHLMLLERMPLTVNGKLDRQALPQPDAA
> SEQ ID NO: 194 -SerD3
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDDGLLPAAQGVSAELYLGGIGLARGY
HRRPGLSATRFVPNPFPGGAGERLYRTGDLARFQADGNIEYIGRIDHQVKVRGFRIELGEIEAALAGLAGVRDAV
VLAHDGVGGTQLVGYVVADSAEDAERLRESLRESLKRHLPDYMVPAHLMLLERMPLTVNGKLDRQALPQPDAA
> SEQ ID NO: 196 -SerD4
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDDGLLPAAQGVSAELYLGGIGLARGY
HNRAALTAERFVPDPFDEQGGRLYRTGDLARYQADGNIEYIGRIDHQVKVRGFRIELGEIEAALAGLAGVRDAVV
LAHDGVGGTQLVGYVVADSAEDAERLRESLRESLKRHLPDYMVPAHLMLLERMPLTVNGKLDRQALPQPDAA
> SEQ ID NO: 198 -SerD5
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDDGLLPAAQGVSAELYLGGIGLARGY
HNRAALTAERFVPDPFDEQGGRLYRTGDLARYRDEGVIEYAGRIDHQVKVRGFRIELGEIEAALAGLAGVRDAVV
LAHDGVGGTQLVGYVVADSAEDAERLRESLRESLKRHLPDYMVPAHLMLLERMPLTVNGKLDRQALPQPDAA
> SEQ ID NO: 200 -SerD6
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDDGLLPAAQGVSAELYLGGIGLARGY
HNRAALTAERFVPDPFDEQGGRLYRTGDLARYRDEGVIEYAGRIDHQVKVRGFRIELGEIEAALAGLAGVRDAVV
LAHDGVGGTQLVGYVVADSAEDAERLRESLRESLKRHLPDYMVPAHLMLLERMPLTVNGKLDRQALPQPDAA
> SEQ ID NO: 202 -SerD7
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DAAEQQQIVRDWNATAADFPGEHCLHSLIEAQVQATPDAPALIFAAEQLSYAQLNARANQLAHRLREAGVGPDVL
VGICVERSVDMVIGLLAIIKAGGAYVPLDPDYPEDRLAYMMQDSGIGLLLTQSALLQGLPVQVQSLCLDQEGDWL
DGYSTANPINLSHPQNLAYVIYTSGSTGKPKGAGNSHRALVNRLHWMQKAYALDGSDTVLQKTPFSFDVSVWEFF
WPLLTGARLAVALPGDHRDPERLVQTIREHQVTTLHFVPSMLQAFLTHPQVEGCNSLRRVVCSGEALPSELAGQV
LKRLPQTGLFNLYGPTEAAIDVTHWTCTKDDVLSVPIGRPIDNLKTHILDDGLLPAAQGVSAELYLGGIGLARGY
HNRAALTAERFVPDPFDEQGGRLYRTGDLARYRDEGVIEYAGRIDHQVKIRGLRIELGEIEAALAGLAGVRDAVV
LAHDGVGGTQLVGYVVADSAEDAERLRESLRESLKRHLPDYMVPAHLMLLERMPLTVNGKLDRQALPQPDAA
> SEQ ID NO: 204 -fhornDl
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGGTIPDLSWYILDRDLNPVPRGAVGELYIGRAGL
ARGYLRRPGLSATRFVPNPFPGGAGERLYRTGDLARFQADGNIEYIGRIDHQVKVRGFRIELGEIEAALAGLAGV
RDAVVLAHDGVGGTQLVGYVVADSAEDAERLRESLRESLKRHLPDYMVPAHLMLLERMPLTVNGKLDRQALPQPD
AA
> SEQ ID NO: 206 -fhornD2
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDRDLNPVPRGAVGELYIGRAGL
ARGYLRRPGLSATRFVPNPFPGGAGERLYRTGDLARFQADGNIEYIGRIDHQVKVRGFRIELGEIEAALAGLAGV
RDAVVLAHDGVGGTQLVGYVVADSAEDAERLRESLRESLKRHLPDYMVPAHLMLLERMPLTVNGKLDRQALPQPD
AA
> SEQ ID NO: 208 -fhornD3
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDAQLNLQPIGVAGELYLGGEGV
ARGYLRRPGLSATRFVPNPFPGGAGERLYRTGDLARFQADGNIEYIGRIDHQVKVRGFRIELGEIEAALAGLAGV
RDAVVLAHDGVGGTQLVGYVVADSAEDAERLRESLRESLKRHLPDYMVPAHLMLLERMPLTVNGKLDRQALPQPD
AA
> SEQ ID NO: 210 - fhornD4
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDAQLNLQPIGVAGELYLGGEGV
ARGYLERPALTAERFVPDPFGKPGSRVYRSGDLTRGQADGNIEYIGRIDHQVKVRGFRIELGEIEAALAGLAGVR
DAVVLAHDGVGGTQLVGYVVADSAEDAERLRESLRESLKRHLPDYMVPAHLMLLERMPLTVNGKLDRQALPQPDA
A
> SEQ ID NO: 212 -fhornD5
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDAQLNLQPIGVAGELYLGGEGV
ARGYLERPALTAERFVPDPFGKPGSRVYRSGDLTRGRPDGVVDYLGRIDHQVKVRGFRIELGEIEAALAGLAGVR
DAVVLAHDGVGGTQLVGYVVADSAEDAERLRESLRESLKRHLPDYMVPAHLMLLERMPLTVNGKLDRQALPQPDA
A
> SEQ ID NO: 214 -fhornD6
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDAQLNLQPIGVAGELYLGGEGV
ARGYLERPALTAERFVPDPFGKPGSRVYRSGDLTRGRPDGVVDYLGRVDHQVKVRGFRIELGEIEAALAGLAGVR
DAVVLAHDGVGGTQLVGYVVADSAEDAERLRESLRESLKRHLPDYMVPAHLMLLERMPLTVNGKLDRQALPQPDA
A
> SEQ ID NO: 216 -fhornD7
SRTTDAVSTIPLADRQQPLALSFAQERQWFLWQLEPESAAYHIPSALRLRGRLDVDALQRSFDSLVARHETLRTR
FRLEGGRSYOOVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DNAERQQILQLWNQSDAGFSAKRLVHELVADRAGETPEAVAVKFGEQQLTYGELDRQANRLAHALIARGVGPEVR
VAIAMPRSAEIMVAFLAVMKAGGVYVPLDIEYPRDRLLYMMQDSRARLLLTHSAVQQRLPIPDGLEALAVDQAQA
WSADDDTAPEVALDGDNLAYVIYTSGSTGMPKGVAVSHGPLVAHIIATGERYETSPADCELHFMSFAFDGSHEGW
MHPLINGASVLIRDDSLWLPEYTYQQMHRHHVTMAVFPPVYLQQLAEHAERDGNPPKVRVYCFGGDAVAQASYDL
AWRALKPTYLFNGYGPTETVVTPLLWKARRGDPCGAVYAPIGTLLGNRSGYVLDAQLNLQPIGVAGELYLGGEGV
ARGYLERPALTAERFVPDPFGKPGSRVYRSGDLTRGRPDGVVDYLGRVDHQVKIRGFRIELGEIEAALAGLAGVR
DAVVLAHDGVGGTQLVGYVVADSAEDAERLRESLRESLKRHLPDYMVPAHLMLLERMPLTVNGKLDRQALPQPDA
A
> SEQ ID NO: 218 -GlyD1
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGGTIPDLSWYILDRDLNPVPRGAVGELYIGRAGLARGYLRR
PGLSATRFVPNPFPGGAGERLYRTGDLARFQADGNIEYIGRIDHQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 220 -GlyD2
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDRDLNPVPRGAVGELYIGRAGLARGYLRR
PGLSATRFVPNPFPGGAGERLYRTGDLARFQADGNIEYIGRIDHQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 222 -GlyD3
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLRR
PGLSATRFVPNPFPGGAGERLYRTGDLARFQADGNIEYIGRIDHQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 224 - GlyD4
SRTTDAVSTIPLADRQQPLALSFAQERQWFLWQLEPESAAYHIPSALRLRGRLDVDALQRSFDSLVARHETLRTR
FRLEGGRSYOOVQPAVSVSIEREQFGEEGLIERIOAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYQADGNIEYIGRIDHQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 226 -GlyD5
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRIDHQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 228 -GlyD6
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKVRGFRIELGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 230 -GlyD7
SRTTDAVSTIPLADRQQPLALSFAQERQWFLWQLEPESAAYHIPSALRLRGRLDVDALQRSFDSLVARHETLRTR
FRLEGGRSYQQVQPAVSVSIEREQFGEEGLIERIQAIVVQPFDLERGPLLRVNLLQLAEDDHVLVLVQHHIVSDG
WSMQVMVEELVQLYAAYSQGLDVVLPALPIQYADYALWQRSWMEAGEKERQLAYWTGLLGGEQPVLELPFDRPRP
ARQSHRGAQLGFELSRELVEAVRALAQREGASSFMLLLASFQALLYRYSGQADIRVGVPIANRNRVETERLIGFF
VNTQVLKADLDGRMGFDELLAQARQRALEAQAHQDLPFEQLVEALQPERNASHNPLFQVLFNHQSEIRSVTPEVQ
LEDLRLEGLAWDGQTAQFDLTLDIQEDENGIWASFDYATDLFDASTVERLAGHWRNLLRGIVANPRQRLGELPLL
DGAESRRMLVEWNDTREAYAADRRVHELFEAQAARTPDASAVESQGRALSYAELNRRANQLARHLRRMGVGPEVL
VGICVEKSVEMLVGLLGILKAGGAYVPLDPAFPAERLAFMAEDAELPVLLTESRLTAVVPPNPTRRTVLLDSDWE
LIAGESGDDLSDTGGGENTAYVIYTSGSTGRPKGVQVSHRALVNFLHSMRGRPGIEAGDTLLAVTTLSFDIAGLE
LYLPLLTGARVVVAGREELSDGALLSERISRSGATVMQATPATWRLLLGAGWRGKSDLRIFCGGEALARELANQL
LEKCAELWNLYGPTETTIWSAIHRVETTEGAVSVGRPIANTRVYVLDKNLRPVPAGVPGELLIGGDGLARGYLNR
PDLTAEKFVPDPFGDAPGARLYRTGDLARYSPGGELEILNRVDTQVKIRGFRIEPGEIEAALAGLAGVRDAVVLA
HDGVGGTQLVGYVVADSAEDAERLRESLRESLKRHLPDYMVPAHLMLLERMPLTVNGKLDRQALPQPDAA
> SEQ ID NO: 232 -NZ_CP020028.1.cluster004_Leu_B
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTEEGSLLLHDFNRTEAEYPKDQTINALFEERAEQRADHPALVWGEQTLTYRELNEQANRLAK
VLRARGVKADDIVAIMTERSMEMVIGILGTLKAGGAYLPIDPNYPEERIHYMLEDSGASMLLTQKHLRDKLTYHG
PIMDVDGEDLKHLELDGHANLQPANKPEDLAYIIYTSGSTGKPKGVMVEHRGIINLWHFFQEQWGVNGSDRMLQF
ASSSFDASVWEMFTILLGGGTLYLVSRDIINNLNEFARFVNENQITIALLPPTYLAGIEPEKLPALKKLVTGGSA
ITKELVTRWKDSVEYMNAYGPSESSVIATAWTYREEDMGYSSVPIGKPIANTRIYIMDEHQKLLPLGAAGEMCVA
GDGLARGYLHRPELTAEKFVVNPYEAGEKLYRTGDLVRWLPDGNIEFLGRIDDQVKIRGFRIELGEIEAQLQKHP
LVQEVAVIAREDKQKEKYLAAYITAEGEPEAEELREQLLQELPDYMVPSSFMQLEHMPMTPSGKIDRKALPEPEA
A
> SEQ ID NO: 234 -NZ_CP020028.1.cluster004_Leu_D
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTEAETSQLLAKFNRTEAEYPKDQTINALFEERAEQRADHPALVWGEQTLTYRELNEQANRLA
KVLRARGVKADDIVAIMTERSMEMVIGILGTLKAGGAYLPIDPNYPEERIHYMLEDSGASMLLTQKHLRDKLTYH
GPIMDVDGEDLKHLELDGHANLQPANKPEDLAYIIYTSGSTGKPKGVMVEHRGIINLWHFFQEQWGVNGSDRMLQ
FASSSFDASVWEMFTILLGGGTLYLVSRDIINNLNEFARFVNENQITIALLPPTYLAGIEPEKLPALKKLVTGGS
AITKELVTRWKDSVEYMNAYGPSESSVIATAWTYREEDMGYSSVPIGKPIANTRIYIMDEHQKLLPLGAAGEMCV
AGDGLARGYLHRPELTAEKFVVNPYEAGEKLYRTGDLVRWLPDGNIEFLGRIDDQVKIRGFRIELGEIEAQLQKH
PLVQEVAVIAREDKQKEKYLAAYITAEGEPEAEELREQLLQELPDYMVPSSFMQLEHMPMTPSGKIDRKALPEPE
AA
> SEQ ID NO: 236 -TycC6 B
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTPEEKQQILAGFNDTAVSYALDKTLHQLFEEQVDKTPDQAALLFSEQSLTYSELNERANRLA
RVLRAKGVGPDRLVAIMAERSPEMVIGILGILKAGGAYVPVDPGYPQERIQYLLEDSNAALLLSQAHLLPLLAQV
SSELPECLDLNAELDAGLSGSNLPAVNQPTDLAYVIYTSGTTGKPKGVMIPHQGIVNCLQWRRDEYGFGPSDKAL
QVFSFAFDGFVASLFAPLLGGATCVLPQEAAAKDPVALKKLMAATEVTHYYGVPSLFQAILDCSTTTDFNQLRCV
TLGGEKLPVQLVQKTKEKHPAIEINNEYGPTENSVVTTISRSIEAGQAITIGRPLANVQVYIVDEQHHLQPIGVV
GELCIGGAGLARGYLNKPELTAEKFVANPFRPGERMYKTGDLVKWRTDGTIEYIGRADEQVKVRGYRIEIGEIES
AVLAYQGIDQAVVVARDDDATAGSYLCAYFVAATAVSVSGLRSHLAKELPAYMIPSYFVELDQLPLSANGKVDRK
ALPKPQAA
> SEQ ID NO: 238 -TycC6_D
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTEAETSQLLAKFNDTAVSYALDKTLHQLFEEQVDKTPDQAALLFSEQSLTYSELNERANRLA
RVLRAKGVGPDRLVAIMAERSPEMVIGILGILKAGGAYVPVDPGYPQERIQYLLEDSNAALLLSQAHLLPLLAQV
SSELPECLDLNAELDAGLSGSNLPAVNQPTDLAYVIYTSGTTGKPKGVMIPHQGIVNCLQWRRDEYGFGPSDKAL
QVFSFAFDGFVASLFAPLLGGATCVLPQEAAAKDPVALKKLMAATEVTHYYGVPSLFQAILDCSTTTDFNQLRCV
TLGGEKLPVQLVQKTKEKHPAIEINNEYGPTENSVVTTISRSIEAGQAITIGRPLANVQVYIVDEQHHLQPIGVV
GELCIGGAGLARGYLNKPELTAEKFVANPFRPGERMYKTGDLVKWRTDGTIEYIGRADEQVKVRGYRIEIGEIES
AVLAYQGIDQAVVVARDDDATAGSYLCAYFVAATAVSVSGLRSHLAKELPAYMIPSYFVELDQLPLSANGKVDRK
ALPKPQAA
> SEQ ID NO: 240 -SrfAC B
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTDREREFLLTGLNPPAQAHETKPLTYWFKEAVNANPDAPALTYSGQTLSYRELDEEANRIAR
RLQKHGAGKGSVVALYTKRSLELVIGILGVLKAGAAYLPVDPKLPEDRISYMLADSAAACLLTHQEMKEQAAELP
YTGTTLFIDDQTRFEEQASDPATAIDPNDPAYIMYTSGTTGKPKGNITTHANIQGLVKHVDYMAFSDQDTFLSVS
NYAFDAFTFDFYASMLNAARLIIADEHTLLDTERLTDLILQENVNVMFATTALFNLLTDAGEDWMKGLRCILFGG
ERASVPHVRKALRIMGPGKLINCYGPTEGTVFATAHVVHDLPDSISSLPIGKPISNASVYILNEQSQLQPFGAVG
ELCISGMGVSKGYVNRADLTKEKFIENPFKPGETLYRTGDLARWLPDGTIEYAGRIDDQVKIRGHRIELEEIEKQ
LQEYPGVKDAVVVADRHESGDASINAYLVNRTQLSAEDVKAHLKKQLPAYMVPQTFTFLDELPLTTNGKVNKRLL
PKPDAA
> SEQ ID NO: 242 -SrfAC_D
AYEQKTTLPKKEAAFAKAFQPTQYRFSLNRTLTKQLGTIASQNQVTLSTVIQTIWGVLLQKYNAAHDVLFGSVVS
GRPTDIVGIDKMVGLFINTIPFRVQAKAGQTFSELLQAVHKRTLQSQPYEHVPLYDIQTQSVLKQELIDHLLVIE
NYPLVEALQKKALNQQIGFTITAVEMFEPTNYDLTVMVMPKEELAFRFDYNAALFDEQVVQKLAGHLQQIADCVA
NNSGVELCQIPLLTEAETSQLLAKLNPPAQAHETKPLTYWFKEAVNANPDAPALTYSGQTLSYRELDEEANRIAR
RLQKHGAGKGSVVALYTKRSLELVIGILGVLKAGAAYLPVDPKLPEDRISYMLADSAAACLLTHQEMKEQAAELP
YTGTTLFIDDQTRFEEQASDPATAIDPNDPAYIMYTSGTTGKPKGNITTHANIQGLVKHVDYMAFSDQDTFLSVS
NYAFDAFTFDFYASMLNAARLIIADEHTLLDTERLTDLILQENVNVMFATTALFNLLTDAGEDWMKGLRCILFGG
ERASVPHVRKALRIMGPGKLINCYGPTEGTVFATAHVVHDLPDSISSLPIGKPISNASVYILNEQSQLQPFGAVG
ELCISGMGVSKGYVNRADLTKEKFIENPFKPGETLYRTGDLARWLPDGTIEYAGRIDDQVKIRGHRIELEEIEKQ
LQEYPGVKDAVVVADRHESGDASINAYLVNRTQLSAEDVKAHLKKQLPAYMVPQTFTFLDELPLTTNGKVNKRLL
PKPDAA

The examples provided herein are provided for the purpose of illustrating specific embodiments and aspects of the invention and are not intended to limit the invention in any way. Persons of ordinary skill can utilise the disclosures and teachings herein to produce other embodiments, aspects, and variations without undue experimentation. All such embodiments, aspects, and variations are considered to be part of this invention.

### EXAMPLES

### Overview and Summary of Experimental Results

Non-ribosomal peptide synthetases (NRPS) are large modular enzymes that govern the synthesis of numerous biotechnologically relevant products. Their mode of action is frequently compared to an assembly line, in which each module acts in a semi-autonomous but coordinated manner to add a specific monomer to a growing peptide chain, unfettered by ribosomal constraints. The modular nature of these systems offers tantalising prospects for synthetic biology, wherein the assembly line is re-engineered at a genetic level to generate a specific or combinatorial modified product. However, although this has clearly been a primary mechanism of natural product diversification throughout evolution, equivalent strategies have proven challenging to implement in the laboratory.

A primary constraint has been the dogmatic assumption that there are two levels of “proof-reading” that govern the selection of an individual amino acid for incorporation into a peptide product. That is proof-reading that not only occurs during the initial selection of that amino acid by the adenylation (A) domain, but also during peptide bond formation, which is catalysed by an adjacent condensation (C) domain. As such, it has been broadly accepted that new products cannot be efficiently generated by substitution of A domains alone, and that the C domain must also be replaced to enable a new amino acid to be incorporated. As shown herein, the inventors have obtained surprising evidence that this is not the case, and that new products can efficiently be generated via a strategy of substituting A domains alone. In accordance with these results, successful recombination strategies and constructs are provided.

### Example 1: Vectors for Substitution of Domains into the Enzyme PvdD

The inventors' previous experiments involved the production of variants of the pvdD gene. The variants were constructed in plasmids and subsequently transformed into a pvdD deletion strain, i.e., a P. aeruginosa strain in which the native pvdD gene had been knocked out. The introduced variants of the pvdD gene were then tested for pyoverdine production when the complemented P. aeruginosa strain was grown in liquid media (Calcott et al. 2014; Calcott et al. 2015). In the current experiments, a series of plasmids were created to perform domain substitutions into PvdD. The plasmids were based on the plasmid pUCP22 with an inserted pBAD promoter, namely pUCP22:pBAD (SEQ ID NO: 3).

The plasmid pUCBAD-SMC (SEQ ID NO: 4) was created to express a pvdD gene lacking the C-A domains in module 2. This plasmid contains SpeI and NotI restriction sites to enable DNA sequences, encoding C-A domains, to be inserted using compatible XbaI and NotI sites. The plasmid pDEC-Lys (SEQ ID NO: 5) contains a copy of the pvdD gene lacking a C domain in module 2, in which the second A domain has been replaced with the Lys A domain from pvdJ. This plasmid contains SpeI and SalI restriction sites to enable DNA sequences, encoding C domains to be inserted, using compatible XbaI and XhoI sites.

The plasmid pDEC-Thr (SEQ ID NO: 6) contains a copy of the pvdD gene lacking a C domain in module 2. This plasmid contains SpeI and SalI restriction sites to enable DNA sequences, encoding C domains, to be inserted using compatible XbaI and XhoI sites. The plasmid pTRN (SEQ ID NO: 7) contains a copy of the pvdD gene lacking the 3′ portion of the C domain in module 2. This plasmid contains SpeI and SalI restriction sites to enable DNA sequences, encoding C domains, to be inserted using compatible SpeI and XhoI sites.

### Example 2: Analysis of Pyoverdine Production

As described previously, domains were introduced into the above-noted plasmids and tested using established methods for analysing pyoverdine production (Calcott et al. 2014; Calcott and Ackerley 2015; Owen et al. 2016). In brief, strains were grown in 200 μL of low salt LB in a 96 well plate. After 24 hours growth at 37° C., 10 μL of this starter culture was used to inoculate 190 μL of M9 media containing 0.1% (w/v) L-arabinose and 4 g/l succinate (pH 7.0). Cultures were grown for 37° C. for 24 hours, centrifuged to pellet bacteria, and then 100 μL of supernatant transferred to a fresh 96 well plate and diluted 2× in fresh M9 media to give a total volume of 200 μL. Absorbance (400 nm) was measured using an EnSpire 2300 Multilabel Reader (Perkin Elmer, Waltham, Mass., USA).

For mass spectrometry analysis, 1 μL of supernatant was mixed with 20 μL of matrix (500 μL acetonitrile, 500 μL ultrapure water, 1 μL trifluoroacetic acid, 10 μg α-Cyano-4-hydroxycinnamic acid). Aliquots of 0.5 μl were spotted in triplicate onto an Opti-TOF® 384 well MALDI plate (Applied Biosystems, Foster City, Calif.) and allowed to dry at room temperature. Spots were analysed using a MALDI TOF/TOF 5800 mass spectrometer (Applied Biosystems) in positive ion mode. Peaks were externally calibrated using cal2 calibration mixture (Applied Biosystems).

### Example 3: DNA Shuffling to Isolate Regions Involved in Substrate Specificity

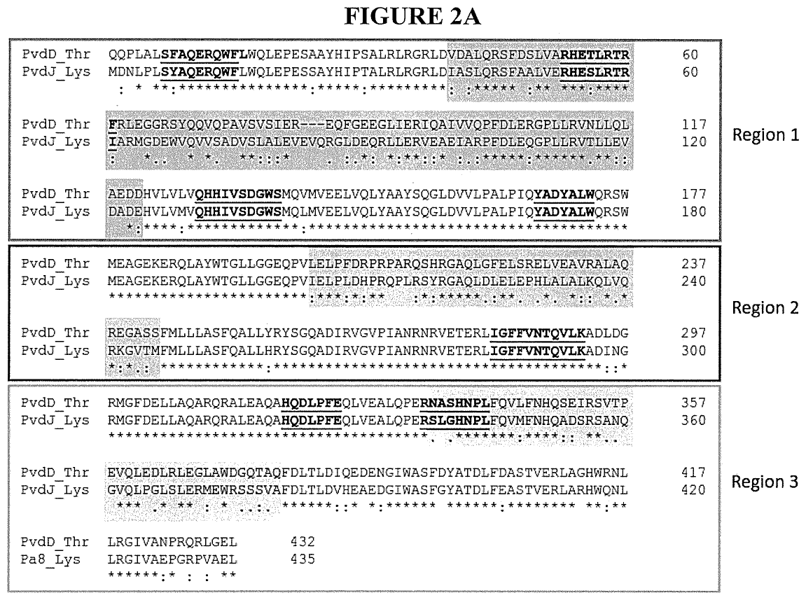

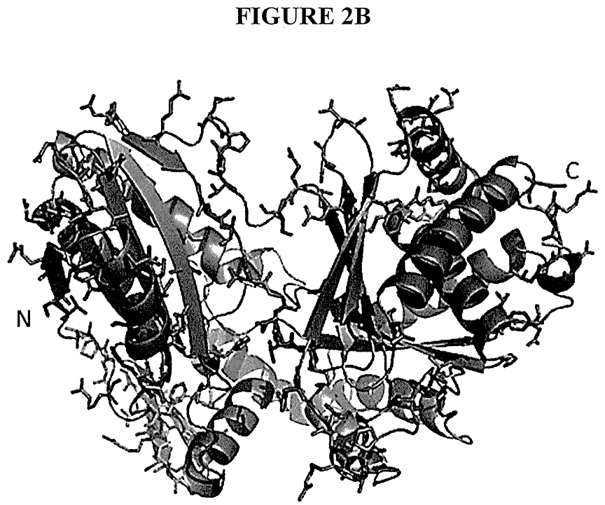

The recombination sites for successful A domain substitution were identified during experiments that attempted to locate the binding site responsible for C domain specificity towards the acceptor substrate. In accordance with the accepted understanding in the field, it had been assumed that C domain specificity at the acceptor site had previously inhibited the production of modified pyoverdines using A domain substitution. To identify residues of NRPS enzymes involved in acceptor site substrate specificity, this experiment shuffled DNA sequences encoding the C domain from the Lys-specific module of PvdJ with the sequence encoding the C domain from the second (Thr-specific) module of PvdD. Based on amino acid sequence identity of the C domains, sequences encoding the Lys specific C domain and the Thr specific C domain were split into three regions (FIG. 2A). A homology model of the C domain from PvdD was created using Raptor X (Kallberg et al. 2012) and showed the three regions could be shuffled effectively with only a minimal number of new amino acid interactions introduced (FIG. 2B). Based on this, DNA shuffling was used to identify which region(s) would allow incorporation of lysine versus threonine into the pyoverdine peptide.

DNA encoding the Lys C domain (SEQ ID NO: 8) and Thr C domain (SEQ ID NO: 10) was generated, as well as DNA encoding the three regions recombined in the six possible combinations (SEQ ID NOs: 12, 14, 16, 18 20, 22). These constructs were restriction digested using XbaI and XhoI and ligated into the SpeI and SalI restriction sites of plasmid pDEC-Lys. When selecting recombination points for this experiment, the downstream point of recombination was located in close proximity to the A1 motif. This meant region 3 of the C domain was always substituted in association with the corresponding linker region, i.e., region 3 from the Thr specific module was always substituted along with the linker region from the Thr specific module and vice versa. This was believed unlikely to be a significant factor because there was no previous evidence that the linker region could be involved in acceptor site specificity.

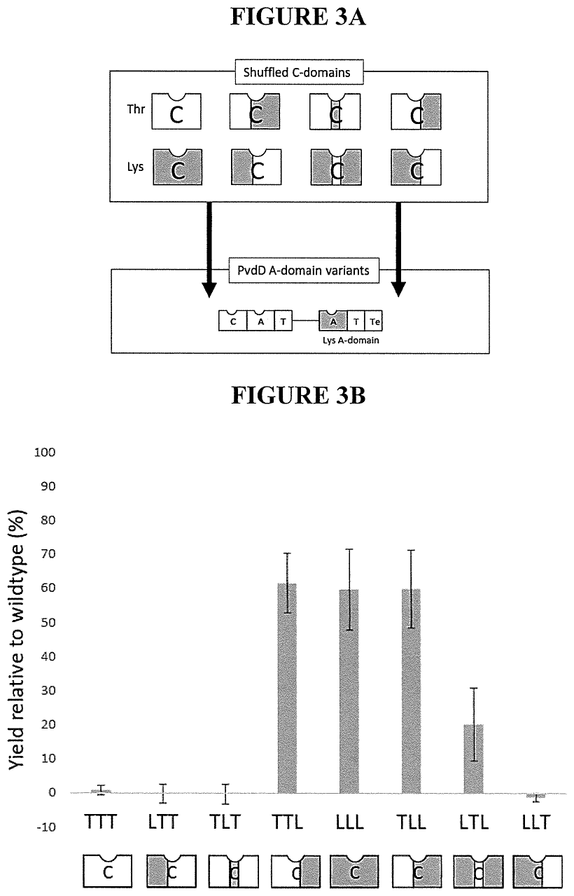

When ligated upstream to the Lys specific A domain in the vector pDEC-Lys (FIG. 3A), it was reasoned that it would be possible to identify regions of the C domain involved with Lys specificity at the acceptor site. The reasoning for this being that only shuffled C domains containing a functional Lys specific acceptor site would produce functional NRPS enzymes. When transformed into the pvdD deletion strain and tested for pyoverdine production, it was found that all shuffled C domain sequences that contained region 3 from a Lys C domain, i.e., the 3′ end of the C domain, were functional with the Lys A domain, and these constructs produced high yields of pyoverdine (FIG. 3B). The pyoverdine was confirmed to contain a terminal Lys residue using MALDI-MS.

The next aim was to identify the precise residues within region 3 of the Lys module that allowed the recombinant C domains to receive Lys as an acceptor substrate. Inspection of the homology model of the C domain from PvdD identified no clear binding pocket formed by the residues differing between the Lys-specific C domain and Thr-specific C domain. To further narrow down the substrate specificity determining residues, it was decided to mutate the residues in region 3 that differed between the C domains and were closest to the catalytic histidine residue.

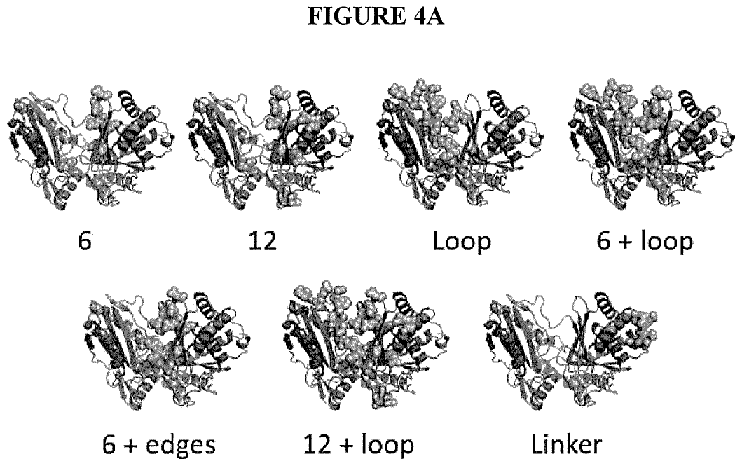

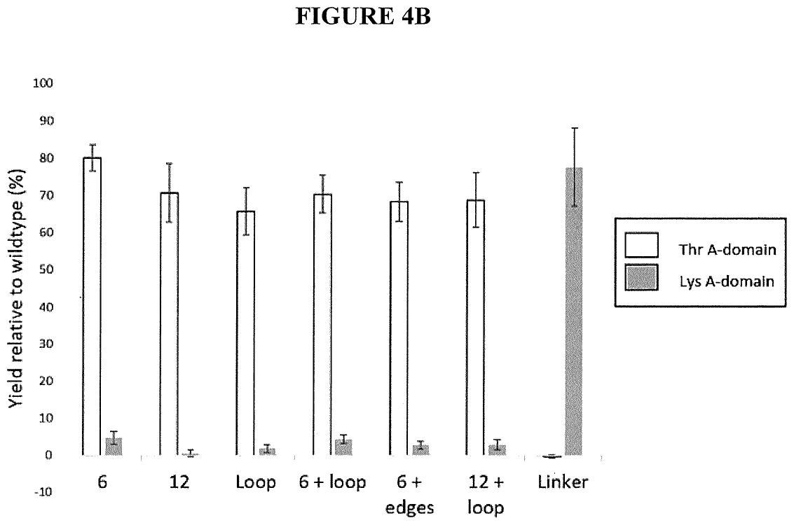

For this, the six or twelve residues were selected that were closest to the catalytic histidine residue as well as a large loop which flexes across the solvent channel of the C domain. These are shown in FIG. 4 as 6, 12, and loop, and the DNA encoding these sequences are SEQ ID NOs. 24, 26 and 28, respectively. These residues were targeted because it was reasoned their close proximity to the catalytic histidine residue meant they were the most likely residues to be involved in determining substrate specificity. However, all three altered C domains resulted in high yield production of pyoverdine when ligated upstream to a Thr C domain in the vector pTRN and transformed into a pvdD deletion strain. In contrast, when the C domains were subsequently ligated upstream to a Lys A domain in the vector pDEC-Lys and transformed into the pvdD deletion strain, there was no pyoverdine produced or very low levels when tested in liquid media.

As mutating the residues closest to the catalytic histidine or loop failed to switch acceptor site specificity, the mutagenesis of the third variable region was expanded. The mutations were introduced to generate substitutions of the six residues closest to the catalytic histidine in combination with mutating the loop (SEQ ID NO: 30), six residues closest to the catalytic histidine in combination with mutating the edges of the loop (SEQ ID NO: 32) and twelve residues closest to the catalytic histidine in combination with mutating the loop (SEQ ID NO: 34). These substitutions collectively targeted all the residues of region 3 that differed between the Thr and Lys C domains but left the linker region as being from the Thr C domain.

As discussed above, the substitutions were made under the assumption that the linker region was unimportant. To test this assumption, a negative control (SEQ ID NO: 36) was designated in which the region 3 of the C domain was unchanged from the Thr C domain but the linker region between the C-A domains was modified to be identical to the one from the Lys C-A domains. When the region 3 alterations were introduced into the vector pTRN and tested in the pvdD deletion strain, they all resulted in high yields of pyoverdine with the exception of the negative control (FIG. 4B). The high yield of pyoverdine when mutating the C domain and loss of function by merely changing the linker region was inconsistent with our assumption that the linker was unlikely to be a significant factor. Instead, this suggested the linker region was a key region in functionality. When the C domains were transferred from the vector pTRN to be upstream to a Lys specific A domain in the vector pDEC-Lys, the results were reversed (FIG. 4B), i.e., the entire Thr C domain combined with only the Lys linker region resulted in high yield production of pyoverdine. The incorporation of Lys into the terminal position of pyoverdine was confirmed by MALDI-MS.

These results were surprising as the Thr C domain being functional with the linker and A domain from a Lys specific module was contrary to the prevailing expectation in the field that C domains play critical roles in acceptor substrate selectivity, and to the expectation that C domain acceptor site specificity had previously caused A domain substitution to be unsuccessful.

### Example 4: Comparison of a Domain Substitutions to C-A Domain Substitutions

The inventors' previous attempts at creating modified pyoverdines were successful when C-A domain substitutions were performed. For this, it was possible to achieve a 3/10 success rate for C-A domain substitutions; however, two of these had relatively low yields of modified pyoverdine (Ackerley and Lamont, 2004; Calcott et al. 2014, Calcott et al. 2015). In light of the current experimental evidence showing that the Lys A domain could be functionally substituted along with the corresponding linker region, it was reasoned the C domain within module 2 of PvdD may exhibit relaxed specificity and therefore other substitutions could be successful. This experiment compared the yield of pyoverdine for our three previously successful C-A domain substitutions to substituting the linker and A domain together.

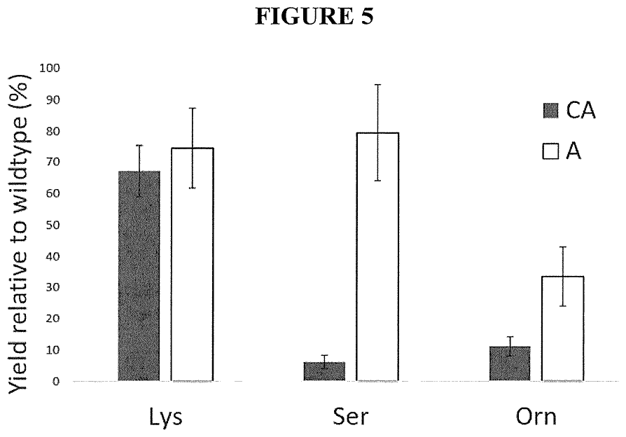

C-A domain substitutions from Lys, Ser and fhOrn specifying modules were introduced into PvdD by restriction digest of DNA encoded by the nucleotide sequences of SEQ ID NOs: 36, 40, 42 using the enzymes XbaI and NotI and ligation into the plasmid pUCBAD-SMC. The genetic regions encoding the linker plus A domain substitutions from Lys, Ser and fhOrn specifying modules were introduced into pvdD by restriction digest of the SEQ ID NOs: 44, 46, 48 using SpeI and XhoI and ligation into the plasmid pTRN. The resulting plasmids were introduced into the pvdD deletion strain and tested for pyoverdine production in liquid media (FIG. 5). In each case the yield of modified pyoverdine produced by the new substitution containing the linker plus A domain was either equal to the C-A domain substitution or greatly increased. This confirmed that not only can substitution of an A domain without a C domain be functional, but that it can result in improved activity compared to C-A domain substitutions.

### Example 5: Performing Additional a Domain Substitutions to Create Modified Pyoverdines

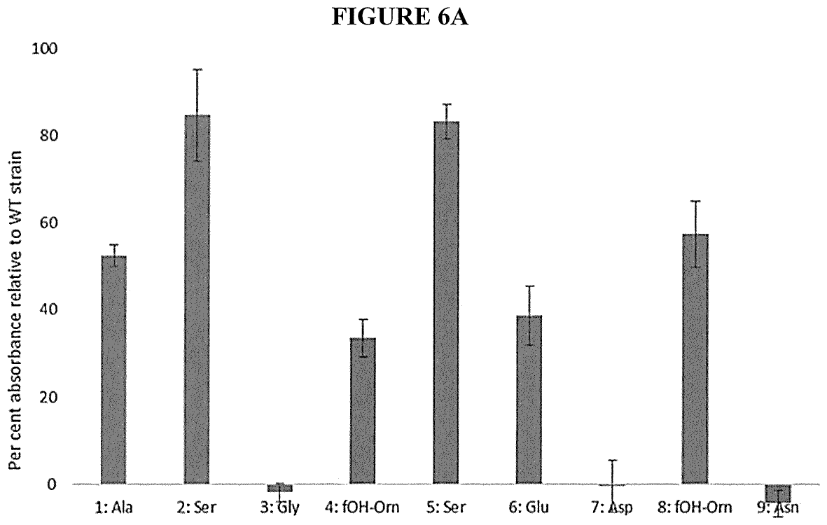

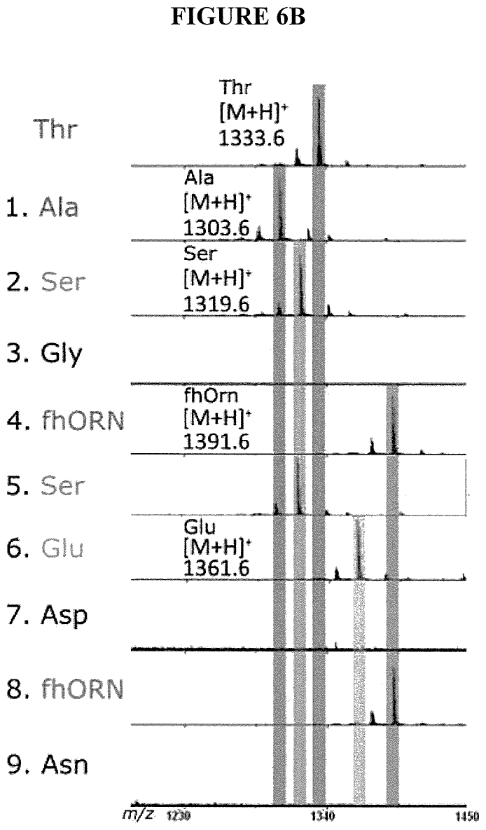

The above-noted experiments demonstrated that substitution of the linker plus A domain increased yield relative to previously successful C-A domain substitutions. To test whether other substitutions could be successful, A domains were selected from 9 modules predicted to activate substrates other than Thr (Table 1). To substitute the linker plus A domain from each of these modules into PvdD, the DNA sequences according to SEQ ID NOs: 52, 54, 56, 58, 60, 62, 64, 66 were synthesised, digested with SpeI and XhoI and ligated into the vector pTRN. When transformed into the pvdD deletion strain, 6/9 of the A domain substitutions yielded modified pyoverdines produced at high yield (FIG. 6). The success rate and yield were both much higher than observed previously using C-A domain substitutions.

TABLE 1
Substrate specificity predictions of A domains substituted into PvdD

| | Specificity predictions from the AntiSMASH | | | | | | |
|---|---|---|---|---|---|---|---|
| | database | | | | | Amino acid identity | |
| | NRPS Predictor2 | Stachelhaus | | | | to Pa11-Thr (%)** | |
| | SVM | code | Minowa | Consensus | Cluster name* | C domain | A domain |
| 1 | ala | ala | ala | ala | CP008696.1.cluster009_CA1 | 66.57 | 51.32 |
| 2 | ser | ser | ser | ser | CP006852.1.cluster006_CA4 | 72.24 | 50.54 |
| 3 | gly | gly | gly | gly | CP011507.1.cluster002_CA1 | 71.94 | 47.58 |
| 4 | hydrophilic | orn | orn | orn | CP003041.1.cluster006_CA2 | 73.43 | 47.01 |
| 5 | ser | ser | ser | ser | AP013068.1.cluster003_CA1 | 58.51 | 50.64 |
| 6 | glu | glu | ser | glu | CP010945.1.cluster006_CA1 | 53.61 | 48.39 |
| 7 | asp, asn, glu, gln, aad | asp | orn | nrp | AM181176.4.cluster005_CA2 | 42.99 | 43.98 |
| 8 | hydrophilic | trp | orn | nrp | CP000680.1.cluster003_CA3 | 53.13 | 47.39 |
| 9 | asp | asn | asp | asp | CP011972.1.cluster002_CA4 | 53.73 | 39.48 |

Shown are the name of each cluster and amino acid identity of C- and A domains to the corresponding domains from module Pa11-Thr.
*Domains were named according to the cluster name from the AntiSMASH database (Blin et al. 2017), and a number based on the order the CA domains appeared in the GBK file.
**C domains were trimmed to the C1 and C7 motifs, and A domains were trimmed to the A1 and A10 motifs inclusive. Domains were aligned using MUSCLE, and the resulting alignment used to calculate percent identity to the corresponding sequence from module 2 of PvdD.

### Example 6: Analysis of Natural Domain Substitution in Pyoverdine Biosynthesis

That the A domain substitutions were greatly improved over the corresponding C-A domain substitutions was surprising and suggested that A domain substitution may be applicable to engineering other NRPS pathways. However, the tight acceptor site specificity originally suggested in the work of Belshaw et al (1999) indicated it may not be feasible for A domain substitution to be performed downstream to C domains with tight specificity.

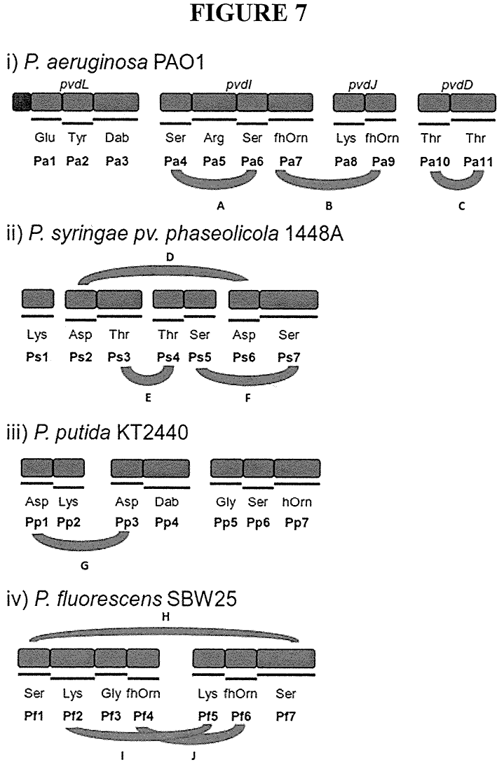

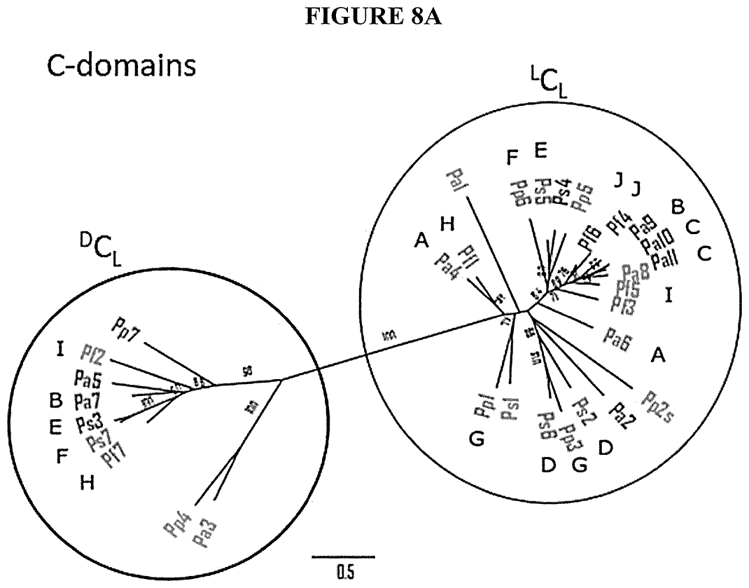

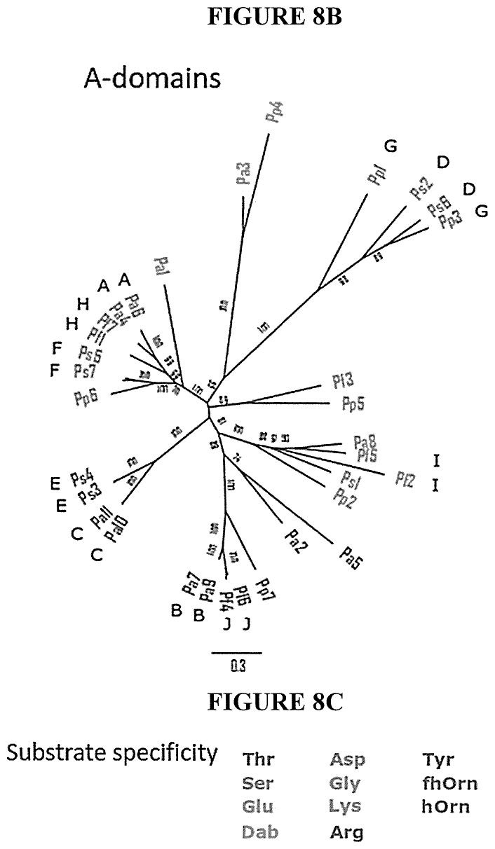

To identify how transferrable the results may be to other NRPS pathways, evolutionary evidence was assessed. Tight acceptor site specificity suggests C- and A domains co-evolve, and it was reasoned that if C domain acceptor site specificity is frequently relaxed then there should be evidence of A domain substitution occurring in nature. To begin with, identification was sought for putative A domain substitution events in the evolution of four pyoverdines (FIG. 7). Distinct maximum likelihood phylogenetic trees were constructed based on the C- and A domains from NRPS enzymes involved in their biosynthesis (FIG. 8). It was reasoned that modules within a single pathway that specify the same substrate, labelled A to J in FIG. 7, may be the result of domain substitution. Besides the instances labelled C, D and I, the C domains from potential domain substitution events were not closely related (FIG. 8A).

The most pronounced cases of this were instances B, E, F and H, in which one module contained a D-amino acid specific C domain and the second contained an L-amino acid specific C domain. In contrast to the phylogenetic tree of C domains, it was found that A domains cluster strongly by substrate specificity (FIG. 8B). Moreover, the A domains from putative domain substitution events were generally closely related to each other. The close relationship between A domains encoding the same substrates and the phylogenetic inconsistencies between C- and A domains provides evidence that A domain substitution has been a driver in the diversification of these pyoverdine biosynthetic pathways.

### Example 7: Evolution of NRPS Diversity in Pseudomonas, Streptomyces and Bacillus Genera

To provide a more global analysis of whether C- and A domains evolve independently, sequences of NRPS domains were analysed from three genera. The sequences for all NRPS gene clusters from the antiSMASH database (Blin et al. 2017) for the genera Pseudomonas, Streptomyces and Bacillus were downloaded. DNA sequences encoding ^{L}C_{L}-A-PCP tridomains were extracted and clustered at 95% identity. A codon-alignment of the centroid nucleotide sequence from each cluster was generated using MUSCLE (Edgar, 2004). Regions of ambiguous alignment were removed using GBLOCK version 0.91b (Castresana, 2000). The default parameters were used for GBLOCK except the minimum number of sequences for a flank position was set equal to 50% of the total sequences, the minimum length of a block was 5, and gap positions were allowed in half of the sequences. This resulted in three alignments, having a total of 528, 465 and 331 sequences for Pseudomonas, Bacillus and Streptomyces species, respectively.

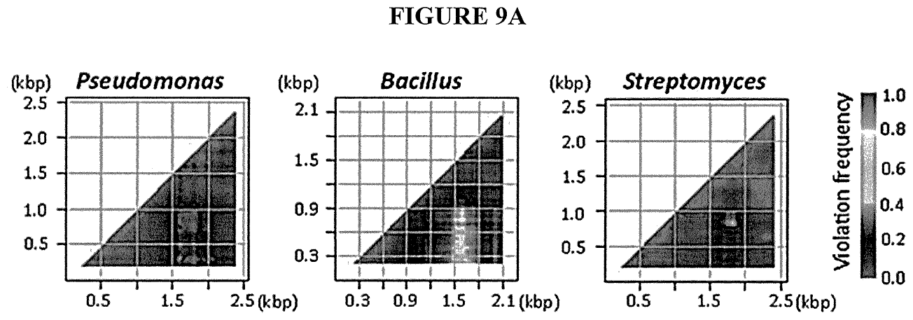

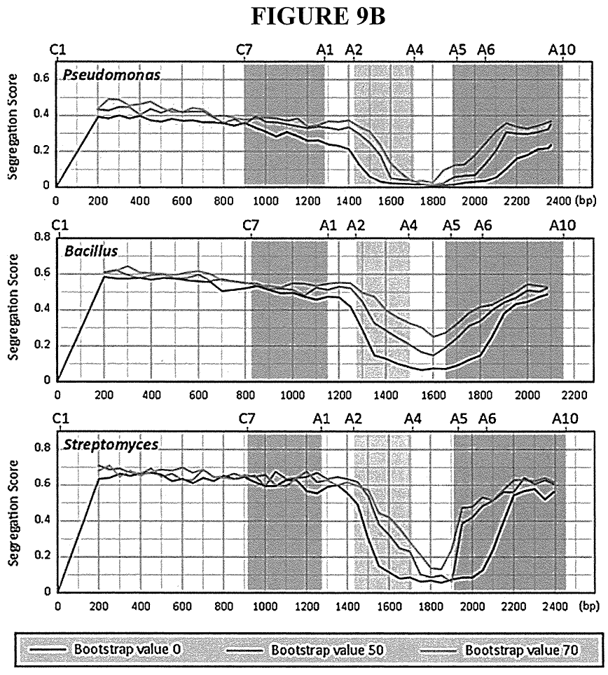

To identify regions where recombination events had likely frequently occurred, the alignments were first analysed using TreeOrderScan (Simmonds, 2006; Simmonds, 2012). Sequences were grouped by the antiSMASH consensus prediction of A domain substrate specificity, and any groups of only a single sequence were removed. Next the nucleotide alignments were split into subalignments of 400 bp at 50 bp intervals and phylogenetic incompatibility matrices created. This found regions of increased phylogenetic incompatibility between A domains and the surrounding domains (FIG. 9A). Segregation analysis of the 400 bp subalignments found this region to be associated with increased clustering according to substrate specificity (FIG. 9B).

Following analysis with TreeOrderScan, recombination analysis of the sequences was performed using RDP4 (Martin et al. 2015), which uses multiple tools to identify putative regions at which DNA sequences have recombined. Default settings were used except sequences were specified as linear, only recombination events detected by at least three methods were considered and alignment consistency was unchecked. A breakpoint distribution plot was created using a 200 bp window and 1,000 permutations. The breakpoint distribution plot identified recombination hotspots located between the C- and A domain, upstream to the A domain binding pocket between the A2 and A4 motifs, and downstream to the binding pocket starting from close to the A5 motif (FIG. 9C).

The recombination hotspot analysis found the largest hotspots to be immediately on each side of the binding pocket, and the region between these hotspots corresponded to the region found to be most strongly segregate by substrate specificity when analysed with TreeOrderScan (FIG. 9A; FIG. 9B). In previous work, our A domain substitutions used recombination points located close to the A1 and A10 motifs. The recombination analysis found the A10 motif to be located within a local recombination hotspot, however the A1 motif was located within a trough between two hotspots. In contrast, the upstream location for recombination used for our successful Lys A domain substitution (labelled X) was centred on a local recombination hotspot; indicating increase levels of recombination in nature at this point. The similar results for Pseudomonas, Bacillus and Streptomyces species are inconsistent with the hypothesis that barriers to A domain substitution cause C- and A domains to co-evolve, and demonstrates that A domain and subdomain substitution have played a role in diversifying NRPS pathways in nature.

### Example 8: Testing A Domain Substitution Versus Partial A Domain Substitution

The above experiment shows A domain substitution occurs in nature, and one of the most striking results was that partial A domain substitution, i.e. substitution of only a region between the A2 and A6 motifs, is particularly favoured in natural evolution. This suggested that partial A domain substitution may be more favourable than substituting the linker and A domain together. To test this, DNA encoding the Ser specific A domain was used (labelled 2 in Table 1) and DNA encoding the Lys specific A domain from PvdJ was used. These two A domains had been shown to function in PvdD in the above experiments, and this experiment aimed to determine whether partial A domain substitution could produce similar yields of modified pyoverdine.

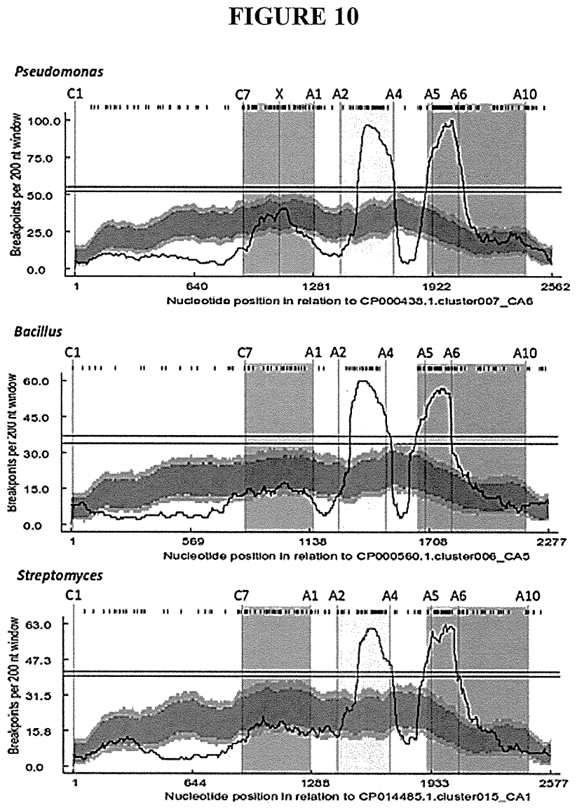

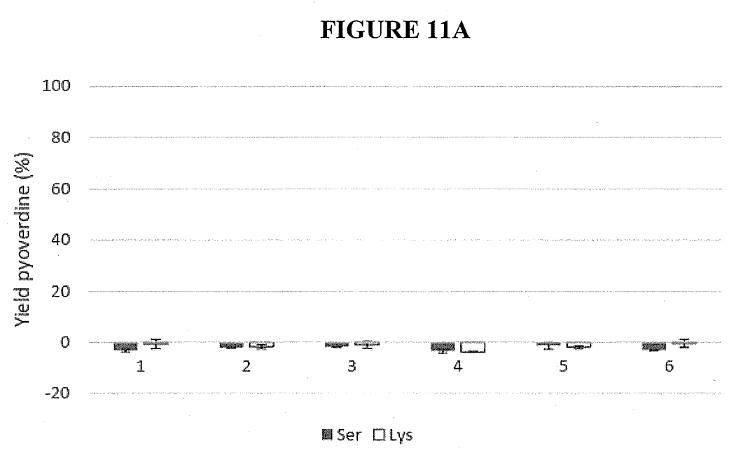

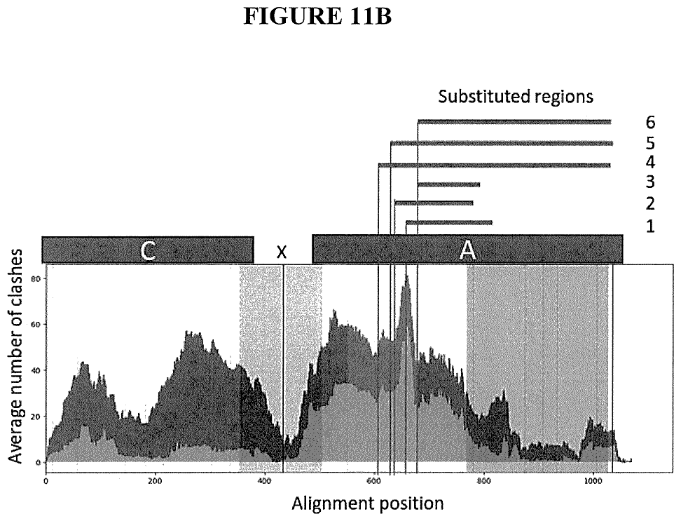

In initial testing, recombination points were selected for partial A domain substitution that were close to the peak of the recombination hotspots identified in FIG. 10. The sequences of SEQ ID NOs: 68 and 70 were for the partial A domain substitution from the Ser and Lys specific A domain. The DNA encoding these sequences was digested with SpeI and XhoI and ligated into pTRN. When transformed into the pvdD deletion strain and tested for pyoverdine production in liquid media, these substitutions resulted in no or very low levels of pyoverdine (FIG. 11A, sample 1). Based on these A domains having been shown to function in pvdD and the evidence of natural recombination occurring most frequently at these points, it was unexpected for pyoverdine production to be so strongly reduced relative to the linker plus A domain substitutions.

To understand why the initial attempt at partial A domain substitution was unsuccessful, the tool SCHEMA (Voigt et al. 2002) was used to analyse the number of perturbations introduced by recombination at each point along C-A domain pairs. Analysis was based on the structure 2VSQ as it was identified as the top template for modelling the C-A domains from the second module of PvdD using the Swiss-Model server (Waterhouse et al. 2018). Sequences were aligned with MUSCLE and then a contact map created with SCHEMA. A python script was then used to calculate the number of clashes using SCHEMA for each potential recombination point between the C-A domains of PvdD and the modules used as a source of A domains in Table 1. A bar graph for single recombination points was generated showing the average number of perturbations per recombination point (FIG. 11B). This showed the upstream recombination point used for our initial partial A domain substitutions (labelled substitution 1 in FIG. 11B) was within a region that introduces a large number of perturbations into the enzyme. In contrast, the site used for successful linker plus A domain substitution was found to be in a structurally favoured position.

Given the discrepancy between recombination hotspots in nature and lack of success for partial A domain substitution, five additional partial A domain substitutions were assessed for both the Ser and Lys specific A domains. The nucleotide sequences of SEQ ID NOs: 72, 74, 76, 78, 80, 82, 84, 86, 88, 90 were generated, digested with SpeI and XhoI and ligated into pTRN. The approximate regions that were substituted are labelled 2 to 6 in FIG. 11B, and were designed to test the optimal upstream recombination points based on our SCHEMA analysis. When the constructs were transformed into the pvdD deletion strain and tested for function, none produced high yields of modified pyoverdines (FIG. 11B). This result showed that substituting the linker plus A domain can result in higher yields of pyoverdine than partial A domain substitution, and that the upstream recombination point for partial A domain substitution may be structurally unfavourable. It may be that the partial A domain recombination that appears to occur with a high degree of frequency in nature reflects a high likelihood of crossover occurring at these locations, however a low proportion of successful outcomes, and hence this strategy may be unsuitable for laboratory-based recombination studies, where a relatively high success rate is required given the impracticality of generating large numbers of artificial constructs with existing methods.

### Example 9: Further Defining the Optimal Regions for Recombination Between C- and a Domains

As the current experiments identified an upstream recombination point that allows successful A domain substitution, the next aim was to test the flexibility in location for placing this recombination point. Whilst keeping the downstream recombination point constant, domain substitutions were performed in which the upstream recombination point was located at four additional locations (FIG. 12A). To create the substitutions, the DNA sequences of SEQ ID NOs: 92, 94, 96, 98, 100, 102, 104, 106, and 141, 143, 145, 147, 149, 151, 153, 155, 157, 159, 161, 163 were digested with XbaI and NotI then ligated into the plasmid pUCBAD-SMC. The plasmids were transformed into the pvdD deletion strain and tested for pyoverdine production in liquid media (FIG. 12B).

Compared with the corresponding Ser and fhOrn C-A domain substitutions created in experiment 3 and Gly and Phe C-A domain substitutions in this experiment, all the A domain recombination points resulted in significantly increased yield of pyoverdine for the substitutions from the Ser-specific module. In addition, the sites ‘X’ and ‘B’ resulted in increased pyoverdine yield for substitutions from the fhOrn-specific module and sites ‘B’ and ‘C’ resulted in an increase for substitutions from the Gly-specifying module. These results show that the upstream recombination site can be varied and still result in high yields of modified pyoverdines.

### Example 10: Demonstration of the Recombination Principles in a Second System: Creation of Dipeptides by a Domain Substitution

The experimental data provided herein demonstrates that A domain substitution can be utilised for modifying pyoverdine, and further, that this can be used to produce greatly increased yield and success rates relative to C-A domain substitution or partial A domain substitution.

The phylogenetic and recombination analysis suggested A domain substitution frequently occurs in nature and therefore the results should be transferable to other NRPS pathways. However, the tight acceptor site substrate specificity originally shown using in vitro assays by Belshaw et al (1999) was contrary to this and suggested some C domains may not allow A domain substitutions to be performed. Two assumptions were made in the study by Belshaw et al. namely: (i) that tethering the substrate to the T domain of Pro-CAT bypasses the A domain; an assumption that could be incorrect if, for instance, the binding of a substrate in the A domain binding pocket is a key step in controlling the direction of biosynthesis and required prior to condensation, and (ii) that the reduced catalytic rates observed in vitro were relevant in the in vivo context.

Taking into account these assumptions as well as the inventor's experiments indicating C domain specificity may not be as stringent as previously believed, experiments were carried out to test the transferability of the current methods using the same NRPS system as Belshaw et al. If either of the Belshaw et al assumptions were incorrect, then it was to be taken that A domain substitution would be broadly applicable to other NRPS pathways including the NRPS system of Belshaw et al that was originally used to infer stringent C domain specificity.

The Phe-ATE/Pro-CAT NRPS system originally used by Belshaw et al comprises the first two modules from the NRPS pathway involved in the biosynthesis of tyrocidine in B. brevis (Belshaw et al. 1999). This two-enzyme system was used to infer stringent acceptor site specificity of the C domain within Pro-CAT that precluded incorporation of L-Leu and L-Phe substrates (Belshaw et al. 1999). An A domain substitution plasmid was made based on pET28a containing the C domain from Pro-CAT, restriction sites to insert alternative A domains, and the T-Te domains from SrfAC (SEQ ID NO: 108). The Te-domain from SrfAC was used as it has previous been shown to enable release of linear peptides from Pro-CAT (Belshaw et al. 1999). The Pro specific A domain from Pro-CAT and five additional A domains were selected to insert into the A domain substitution construct. The five additional domains are represented by SEQ ID NOs: 109, 111, 113, 115, 117, 119, and included the Leu specific A domain from SrfAC, three additional Leu specific A domains and a Phe specific A domain. The A domain from SrfAC was of particular interest because the crystal structure of this module has been used to suggest that C-A domains form a tight interface which may prohibit A domain substitution (Tanovic et al. 2008).

The A domains exhibited low sequence identity to the A domain from Pro-CAT, ranging from 40.4% to 47.6% amino acid identity (Table 2). As such this experiment tests the main features that have been understood as prohibiting A domain substitution. In particular, it was assessed whether it was possible to use the C domain that was originally concluded to show tight acceptor site specificity and the A domain showing C- and A domains form a tight interface, and to substitute A domains that exhibited low sequence identity. These experiments were considered to be a good test of the limits of A domain substitution according to the disclosed methods, as according to the conventional understanding in the field, it should not have been possible to use the described approach to generate novel products.

TABLE 2
Substrate specificity predictions of A domains substituted into ProCAT

| | | | Amino acid identity to | |
|---|---|---|---|---|
| | Specificity predictions | | Pa11-Thr (%)** | |
| | using Stachelhaus code | Cluster name* | C domain | A domain |
| 2 | Leu | TycC6_Leu | 22.98 | 43.11 |
| 3 | Leu | SrfAC_Leu | 44.08 | 40.35 |
| 4 | Phe | NZ_CP021920.1.cluster002_Phe_CA3 | 22.98 | 46.33 |
| 5 | Leu | NZ_CP020028.1.cluster004_Leu_CA1 | 43.07 | 47.02 |
| 6 | Leu | NZ_CM000756.1.cluster012_Leu_CA1 | 21.82 | 47.64 |

Shown are the name of each cluster, and amino acid identity of C and A domains to the corresponding domains from ProCAT.
*Domains were named according to the cluster name from the AntiSMASH database (Blin et al. 2017).
**C domains were trimmed to the C1 and C7 motifs, and A domains were trimmed to the A1 and A10 motifs inclusive. Domains were aligned using muscle, and the resulting alignment used to calculate percent identity to the corresponding sequence from ProCAT.

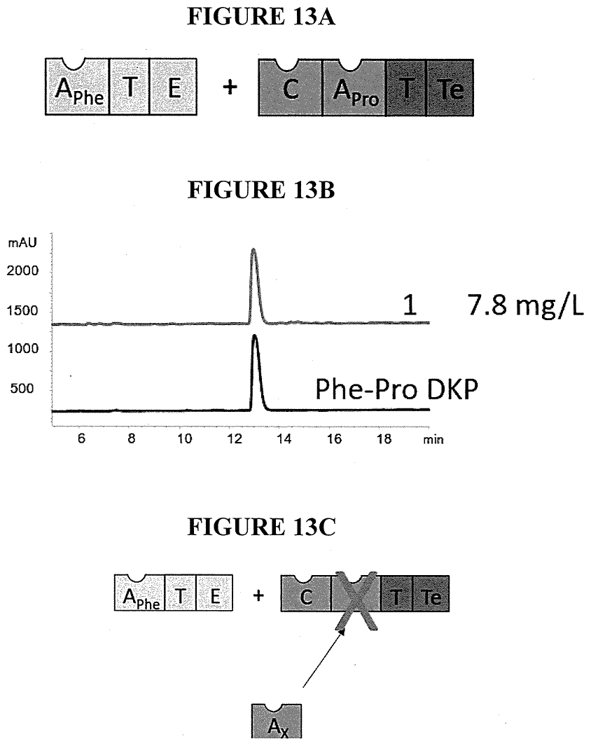

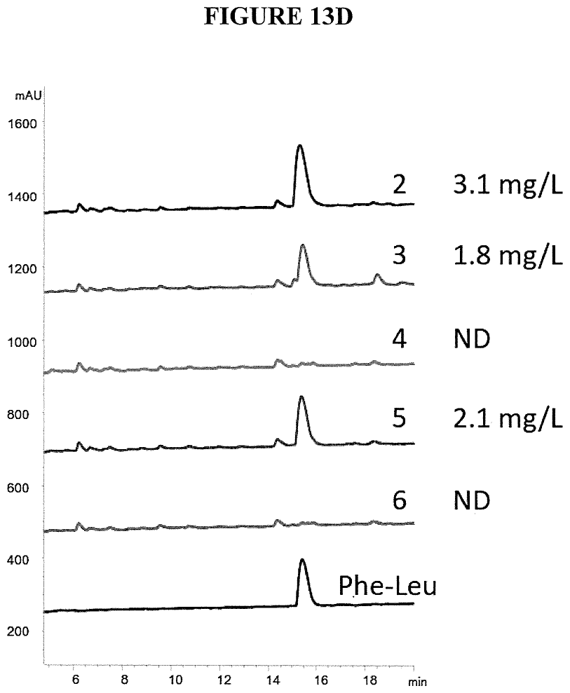

The DNA sequences of SEQ ID NOs: 109, 111, 113, 115, 117, 119, were digested using NheI and NotI, then ligated into the compatible SpeI and NotI restriction sites of the vector pET28:ProC-TTe (SEQ ID NO: 108). The resulting Pro-CATTe constructs were transformed into a BAP1 strain of E. coli (Pfeifer et al. 2001) along with a second plasmid containing Phe-ATE (SEQ ID NO: 121). The strains were grown for 24 hours in 10 mL of M9, extracted and analysed using established protocols (Gruenewald et al. 2004). The production of dipeptides was quantified using HPLC and absorbance at 214 nm (FIG. 13), and confirmed using mass spectrometry. This found that the control Pro A domain substitution strain synthesised D-Phe-L-Pro DKP at 7.8 mg/L. This compares favourably to previous reports (Gruenewald et al. 2004).

Dipeptide production was able to be quantified for three of the Leu A domain substitutions and these ranged from 1.81 mg/L to 3.06 mg/L, i.e. only a slight reduction in product yield relative to the control, indicating relatively effective substitutions. The strain containing the A domain from SrfAC was the A domain having the lowest sequence identity to the A domain from Pro-CAT, and was found to produce D-Phe-L-Leu at 1.81 mg/L. The most functional A domain substitution contained the A domain from TycC6 and produced 3.06 mg/L of D-Phe-L-Leu. This A domain had the second lowest sequence identity to the A domain from ProCAT, showing being closely related to the original A domain is not essential for activity. Overall, the 3/5 success rate and high yield of dipeptides shows that, surprisingly, acceptor site specificity within the Phe-ATE/Pro-CAT NRPS system is not a barrier to using A domain substitution for the in vivo production of alternative peptides.

In summary, the current experiments provide compelling evidence that the C domain proofreading hypothesis stated by Belshaw et al 1999 and Ehmann et al 2000 is not a barrier to successful domain substitution. As shown herein, it has been demonstrated that novel non-ribosomal peptides can be generated with high success rates and fermentation yields that are frequently close to those of the native products, via A domain substitution. In relation to this is the identification of novel recombination boundaries that minimise the number of steric clashes and other incompatibilities with nearby residues. Prior to the experimental evidence provided herein, it had only been possible to generate functional recombinant pyoverdine NRPS enzymes when using A domains that activate the same amino acid, i.e., “synonymous” A domains that do not make any change to the final non-ribosomal peptide. Attempts by other researchers at producing altered peptides by A domain substitution have typically included the complete T domain or part of the T domain, and either only worked in vitro or else produced compounds at low yields (i.e., at only a few percent relative to the unmodified NRPS) in vivo that any modified compounds could only be detected by extremely sensitive techniques such as mass spectrometry. The disclosed enzymes and methods provide a substantial improvement on previous efforts.

### Example 11: Additional Testing of the X, B and D Recombination Sites

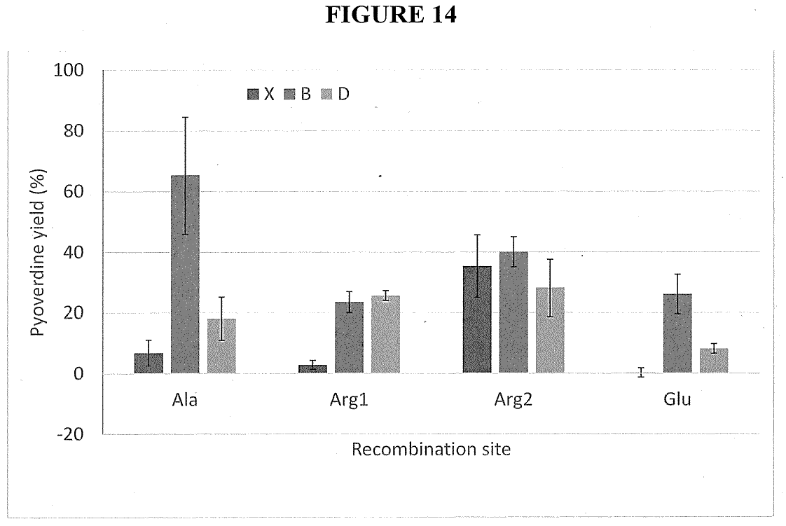

Assessment of the recombination sites tested in Example 9 identified the region stretching from recombination sites ‘X’ to ‘D’ as being the most amenable sites for substitution, with ‘X’, ‘B’ and ‘D’ each generating modified pyoverdine in three out of four cases, and ‘C’ in two out of four cases. To further assess the suitability of these sites for domain substitution, four additional A domains were selected and substituted into PvdD using the recombination sites ‘X’, ‘B’ and ‘D’. The four additional domains are represented by SEQ IDs Nos: 165, 167, 169, 171, 173, 175, 177, 179, 181, 183, 185, 187 and include an Ala, a Glu and two Arg specifying domains, referred to here as Arg1 and Arg2. To create these substitutions, the DNA sequences of SEQ ID NOs: 165, 167, 169, 171, 173, 175, 177, 179, 181, 183, 185, 187 were digested with XbaI and NotI and ligated into the plasmid pUCBAD-SMC. The plasmids were transformed into the pvdD deletion strain and tested for pyoverdine production in liquid media (FIG. 14). Of the three recombination sites tested, the ‘B’ site consistently resulted in the highest yields of modified pyoverdine for each A-domain substitution.

That substitutions using the ‘B’ site resulted in improved yield in the pvdD system suggested production of dipeptides in the PheATE-ProCATTe system could also be improved using the ‘B’ recombination site. To confirm the utility of the ‘X’, ‘B’ and ‘D’ recombination sites, and compare the relevant dipeptide yields generated by using each site, substitutions were made using the PheATE/ProCATTe system from Example 10. The three Leu-specifying A domains that previously generated a D-Phe-L-Leu dipeptide product when substituted at the ‘X’ site (FIG. 13D) were selected for additional testing at the ‘B’ and ‘D’ sites. The three Leu specifying A-domains included the A-domain from SrfAC, the A-domain from TycC module 6 and the A-domain from NZ_CP020028.1.cluster004 and are represented by SEQ ID NOs:231, 233, 235, 237, 239, 241.

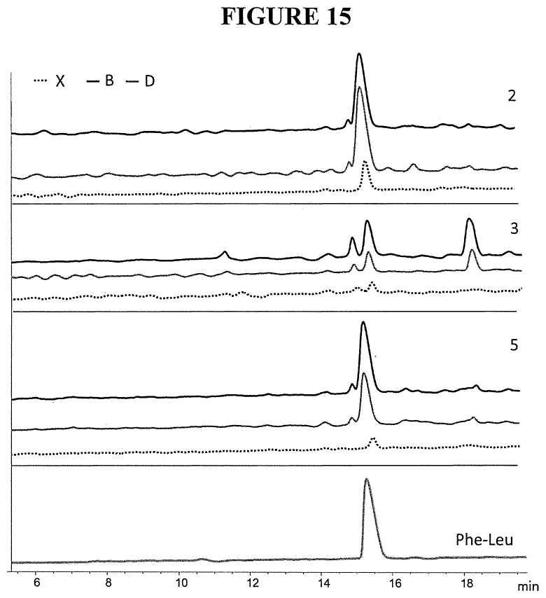

The DNA sequences of SEQ ID NOs: 231, 233, 235, 237, 239, 241 were digested with the restriction enzymes HindIII and NotI, then ligated into the compatible HindIII and NotI restriction sites of the vector pET28:ProC-TTe (SEQ ID NO: 108). The resulting Pro-CATTe constructs were transformed into a BAP1 strain of E. coli (Pfeifer et al. 2001) along with a second plasmid containing Phe-ATE (SEQ ID NO: 121). The strains were grown for 24 hours at 30° C. in 5 mL of M9 medium, extracted, and the relative levels of dipeptide production analysed using established protocols (Gruenewald et al. 2004), i.e. production of dipeptides was quantified using HPLC and absorbance at 214 nm (FIG. 15). All variants produced the D-Phe-L-Leu dipeptide product, and the ‘B’ site was identified as the preferred site based on improved yield relative to the ‘X’ site and having the highest yield in 2/3 of the cases tested.

### Example 12: Testing the Effects of Modifying Downstream Recombination Sites

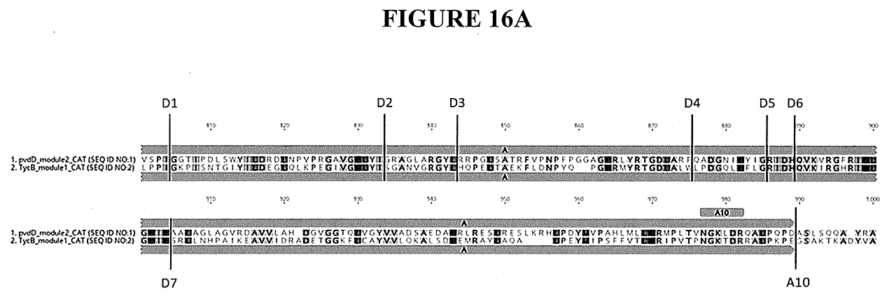

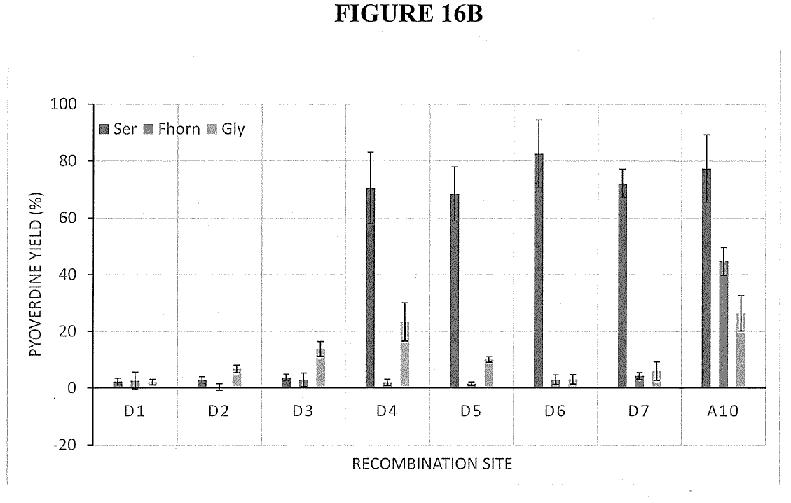

The above experiments used a downstream recombination site close to the A10 motif of the A domain. To test the tolerance of the downstream recombination sites, the downstream recombination site used for A domain substitution was modified while keeping the upstream ‘B’ site constant. The modules specifying Ser, fhOrn and Gly from Example 9 were chosen and a total of seven additional locations were selected to test the downstream recombination sites (FIG. 16A). To create substitutions, the DNA sequences of SEQ ID NOs: 189, 191, 193, 195, 197, 199, 200, 201, 203, 205, 207, 209, 211, 213, 215, 217, 219, 221, 223, 225, 227, 229 were digested with XbaI and NotI and ligated into the plasmid pUCBAD-SMC. The plasmids were transformed into the pvdD deletion strain and tested for pyoverdine production in liquid media (FIG. 16B). It was observed that altering the downstream recombination site resulted in successful production of modified pyoverdines for 13/24 substitutions, and that the ‘D7’ and ‘A10’ sites were the most optimal downstream recombination sites, each enabling production of modified pyoverdines in 3/3 cases tested.

### REFERENCES

Ackerley D F, Lamont I L (2004) Characterization and genetic manipulation of peptide synthetases in Pseudomonas aeruginosa PAO1 in order to generate novel pyoverdines. Chem Biol 11:971-980.
Baltz R H (2011) Function of MbtH homologs in nonribosomal peptide biosynthesis and applications in secondary metabolite discovery. J Ind Microbiol Biotechnol 38:1747-1760.
Baltz R H (2014) Combinatorial biosynthesis of cyclic lipopeptide antibiotics: a model for synthetic biology to accelerate the evolution of secondary metabolite biosynthetic pathways. ACS Synth Biol 3:748-758.
Baltz R H (2018) Synthetic biology, genome mining, and combinatorial biosynthesis of NRPS-derived antibiotics: a perspective. J Ind Microbiol Biotechnol 45:635-649.
Belshaw P J, Walsh C T, Stachelhaus T (1999) Aminoacyl-CoAs as probes of condensation domain selectivity in nonribosomal peptide synthesis. Science 284:486-489.
Bloudoff K, Alonzo D A, Schmeing T M (2016) Chemical probes allow structural insight into the condensation reaction of nonribosomal peptide synthetases. Cell Chem Biol 23:331-339.
Bloudoff K, Schmeing T M (2017) Structural and functional aspects of the nonribosomal peptide synthetase condensation domain superfamily: discovery, dissection and diversity. BBA—Proteins and Proteomics 1865 (2017) 1587-1604
Blin K, Medema M H, Kottmann R, Lee S Y, Weber T. (2017). The antiSMASH database, a comprehensive database of microbial secondary metabolite biosynthetic gene clusters. Nucleic Acids Res, 45, D555-D559.
Bozhüyük K A J, Fleischhacker F, Linck A, Wesche F, Tietze A, Niesert C P, Bode H B (2018) De novo design and engineering of non-ribosomal peptide synthetases. Nat Chem 10:275-281.
Bozhüyük K A J, Linck A, Tietze A, Kranz J, Wesche F, Nowak S, Fleischhacker F, Shi Y N, Grün P, Bode H B (2019) Modification and de novo design of non-ribosomal peptide synthetases using specific assembly points within condensation domains. Nat Chem 11:653-661.
Brown A S, Calcott M J, Owen J G, Ackerley D F (2018) Structural, functional and evolutionary perspectives on effective re-engineering of non-ribosomal peptide synthetase assembly lines. Nat Prod Rep 35:1210-1228.
Bush K (2012) Improving known classes of antibiotics: an optimistic approach for the future. Curr Opin Pharmacol 12:527-534.
Caboche S, Leclère V, Pupin M, et al. (2010) Diversity of monomers in nonribosomal peptides: towards the prediction of origin and biological activity. J Bacteriol 192:5143-5150.
Calcott M J, Owen J G, Lamont I L, Ackerley D F (2014) Biosynthesis of novel pyoverdines by domain substitution in a nonribosomal peptide synthetase of Pseudomonas aeruginosa. Appl Environ Microbiol 80:5723-5731.
Calcott M J, Ackerley D F (2015) Portability of the thiolation domain in recombinant pyoverdine non-ribosomal peptide synthetases. BMC Microbiol 15:162.
Caradec T, Pupin M, Vanvlassenbroeck A, et al. (2014) Prediction of monomer isomery in florine: a workflow dedicated to nonribosomal peptide discovery. PLoS ONE 9:e85667.
Castresana J. (2000) Selection of conserved blocks from multiple alignments for their use in phylogenetic analysis. Molecular Biology and Evolution. 17:540-552.
Chen C-Y, Georgiev I, Anderson A C, Donald B R (2009) Computational structure-based redesign of enzyme activity. Proc Natl Acad Sci USA 106:3764-3769.
Chiocchini C, Linne U, Stachelhaus T (2006) In vivo biocombinatorial synthesis of lipopeptides by com domain-mediated reprogramming of the surfactin biosynthetic complex. Chem Biol 13:899-908.
Crusemann M, Kohlhaas C, Piel J (2013) Evolution-guided engineering of nonribosomal peptide synthetase adenylation domains. Chem Sci 4:1041-1045.
Doekel S, Marahiel M A (2000) Dipeptide formation on engineered hybrid peptide synthetases. Chem Biol 7:373-384.
Doekel S, Coëffet-Le Gal M F, Gu J Q, Chu M, Baltz R H, Brian P. 2008. Non-ribosomal peptide synthetase module fusions to produce derivatives of daptomycin in Streptomyces roseosporus. Microbiology 154:2872-2880.
Duerfahrt T, Doekel S, Sonke T, et al. (2003) Construction of hybrid peptide synthetases for the production of α-l-aspartyl-l-phenylalanine, a precursor for the high-intensity sweetener aspartame. Eur J Biochem 270:4555-4563.
Edgar R C (2004) MUSCLE: a multiple sequence alignment method with reduced time and space complexity. BMC Bioinformatics 5:113.
Ehmann D E, Trauger J W, Stachelhaus T, Walsh C T (2000) Aminoacyl-SNACs as small-molecule substrates for the condensation domains of nonribosomal peptide synthetases. Chem Biol 7:765-772.
Eppelmann K, Stachelhaus T, Marahiel M A (2002) Exploitation of the Selectivity-Conferring Code of Nonribosomal Peptide Synthetases for the Rational Design of Novel Peptide Antibiotics. Biochemistry 41:9718-9726.
Felnagle E A, Jackson E E, Chan Y A, et al. (2008) Nonribosomal peptide synthetases involved in the production of medically relevant natural products. Mol Pharm 5:191-211.
Fischbach M A, Lai J R, Roche E D, et al. (2007) Directed evolution can rapidly improve the activity of chimeric assembly-line enzymes. Proc Natl Acad Sci USA 104:11951-11956.
Fischbach M A, Walsh C T, Clardy J (2008) The evolution of gene collectives: How natural selection drives chemical innovation. Proc Natl Acad Sci USA 105:4601-4608.
Gruenewald S, Mootz H D, Stehmeier P, Stachelhaus T (2004) In vivo production of artificial nonribosomal peptide products in the heterologous host Escherichia coli. Appl Environ Microbiol 70:3282-3291.
Hahn M, Stachelhaus T (2004) Selective interaction between nonribosomal peptide synthetases is facilitated by short communication-mediating domains. Proc Natl Acad Sci USA 101:15585-15590.
Hur G H, Vickery C R, Burkart M D (2012) Explorations of catalytic domains in non-ribosomal peptide synthetase enzymology. Nat Prod Rep 29:1074-1098.
Kallberg M, Wang H, Wang S, Peng J, Wang Z, Lu H, Xu J (2012) Template-based protein structure modeling using the RaptorX web server. Nat Protoc 7:1511-1522.
Kirschning A, Hahn F (2012) Merging chemical synthesis and biosynthesis: a new chapter in the total synthesis of natural products and natural product libraries. Angew Chem Int Ed Engl 51:4012-4022.
Kries H, Niquille D L, Hilvert D (2015) A subdomain swap strategy for reengineering nonribosomal peptides. Chem Biol 22:640-648.
Lin K, Simossis V A, Taylor W R, Heringa J (2005) A simple and fast secondary structure prediction method using hidden neural networks. Bioinformatics 21:152-159.
Linne U, Doekel S, Marahiel M A (2001) Portability of epimerization domain and role of peptidyl carrier protein on epimerization activity in nonribosomal peptide synthetases. Biochemistry 40:15824-15834.
Marahiel M A, Stachelhaus T, Mootz H D (1997) Modular peptide synthetases involved in nonribosomal peptide synthesis. Chem. Rev. 97: 2651-2673.
Martin D P, Murrell B, Golden M, Khoosal A and Muhire B. (2015). RDP4: Detection and analysis of recombination patterns in virus genomes. Virus Evol, 1.
Mootz H, Schwarzer, Marahiel M A (2000) Construction of hybrid peptide synthetases by module and domain fusions. Proc Natl Acad Sci USA 11:5848-5853.
Nguyen K T, Ritz D, Gu J-Q, et al. (2006) Combinatorial biosynthesis of novel antibiotics related to daptomycin. Proc Natl Acad Sci USA 103:17462-17467.
O'Connell K M G, Hodgkinson J T, Sore H F, et al. (2013) Combating multidrug-resistant bacteria: current strategies for the discovery of novel antibacterials. Angew Chem Int Ed Engl. doi: 10.1002/anie.201209979 [Epub ahead of print].
Owen J G, Calcott M J, Robins K J, D F Ackerley. (2016). Generating functional recombinant NRPS enzymes in the laboratory setting via peptidyl carrier protein engineering. Cell Chemical Biology. 23(11): 1395-1406.
Pfeifer B A, Admiraal S J, Gramajo H, Cane D E, Khosla C. (2001) Biosynthesis of complex polyketides in a metabolically engineered strain of E. coli. Science. 291(5509): 1790-2.
Rausch C, Weber T, Kohlbacher O, et al. (2005) Specificity prediction of adenylation domains in nonribosomal peptide synthetases (NRPS) using transductive support vector machines (TSVMs). Nucleic Acids Res 33:5799-5808.
Rausch C, Hoof I, Weber T, Wohlleben W, Huson D H (2007) Phylogenetic analysis of condensation domains in NRPS sheds light on their functional evolution. BMC Evol Biol 7:78.
Rottig M, Medema M H, Blin K, et al. (2011) NRPSpredictor2—a web server for predicting NRPS adenylation domain specificity. Nucleic Acids Res 39:W362-W367.
Schneider A, Stachelhaus T, Marahiel M A (1998) Targeted alteration of the substrate specificity of peptide synthetases by rational module swapping. Mol Gen Genet 257:308-318.
Sieber S A, Marahiel M A (2005) Molecular mechanisms underlying nonribosomal peptide synthesis: approaches to new antibiotics. Chem Rev 105:715-738.
Simmonds P. (2006). Recombination and selection in the evolution of picornaviruses and other mammalian positive-stranded RNA viruses. J Virol. 80:11124-11140.
Simmonds P. (2012) SSE: a nucleotide and amino acid sequence analysis platform. BMC Res Notes. 5:50.Stachelhaus T, Mootz H D, Marahiel M A (1999) The specificity-conferring code of adenylation domains in nonribosomal peptide synthetases. Chem Biol 6:493-505.
Stachelhaus T, Schneider A, Marahiel M A (1995) Rational design of peptide antibiotics by targeted replacement of bacterial and fungal domains. Science 269:69-72.
Stein T, Vater J, Kruft V, et al. (1994) Detection of 4′-phosphopantetheine at the thioester binding site for L-valine of gramicidinS synthetase 2. FEBS Lett 340:39-44.
Stevens B W, Lilien R H, Georgiev I, et al. (2006) Redesigning the PheA Domain of Gramicidin Synthetase Leads to a New Understanding of the Enzyme's Mechanism and Selectivity. Biochemistry 45:15495-15504.
Strieker M, Tanovic A, Marahiel M A. (2010). Nonribosomal peptide synthetases: structures and dynamics. Curr Opin Struct Biol. 20, 234-240.
Süssmuth R D, Mainz A. (2017) Nonribosomal peptide synthesis-principles and prospects. Angew. Chem. Int. Ed. 56:3770-3821
Tanovic A, Samel S A, Essen L-O, Marahiel M A (2008) Crystal structure of the termination module of a nonribosomal peptide synthetase. Science 321:659-663.
Villiers B, Hollfelder F (2011) Directed evolution of a gatekeeper domain in nonribosomal peptide synthesis. Chem Biol 18:1290-1299.
Waterhouse A, Bertoni M, Bienert S, Studer G, Tauriello G, Gumienny R, Heer F T, de Beer T A P, Rempfer C, Bordoli L, Lepore R, Schwede T (2018) SWISS-MODEL: homology modelling of protein structures and complexes. Nucleic Acids Res 46:W296-W303.
Winn M, Fyans J K, Zhuo Y, Micklefield J (2016) Recent advances in engineering nonribosomal peptide assembly lines. Nat Prod Rep 33:317-347.
Yakimov M M, Giuliano L, Timmis K N, Golyshin P N (2000) Recombinant acylheptapeptide lichenysin: high level of production by Bacillus subtilis cells. J Mol Microbiol Biotechnol 2:217-224.
Zhang K, Nelson K M, Bhuripanyo K, et al. (2013) Engineering the substrate specificity of the DhbE adenylation domain by yeast cell surface display. Chem Biol 20:92-101.
Zhou Z, Lai J R, Walsh C T (2007) Directed evolution of aryl carrier proteins in the enterobactin synthetase. Proc Natl Acad Sci USA 104:11621-11626.

See also:
Finking R and M A Marahiel (2004) Biosynthesis of nonribosomal peptides, Annu Rev Microbiol 58: 453-488.
Challis G L, and Naismith J H (2004) Structural aspects of non-ribosomal peptide biosynthesis. Curr Opin Struct Biol 14(6): 748-756.
Marahiel M A and L O Essen (2009) Nonribosomal peptide synthetases: Mechanistic and structural aspects of essential domains. Methods in Enzymology 458: 337-351.
Cox, C D, K L Rinehart, Jr, M L Moore, and J C Cook, Jr (1981) Pyochelin: novel structure of an iron-chelating growth promoter for Pseudomonas aeruginosa. Proc Natl Acad Sci USA 78:4256-4260.
Ankenbauer, R, S Sriyosachati, and C D Cox (1985) Effects of siderophores on the growth of Pseudomonas aeruginosa in human serum and transferrin. Infect Immun 49:132-140.
Meyer, J M, A Stintzi, D De Vos, P Cornelis, R Tappe, K Taraz, and H Budzikiewicz (1997) Use of siderophores to type pseudomonads: the three Pseudomonas aeruginosa pyoverdine systems. Microbiology 143:35-43.
Ackerley, D F, T T Caradoc-Davies, and I L Lamont (2003) Substrate specificity of the nonribosomal peptide synthetase PvdD from Pseudomonas aeruginosa. J Bacteriol 185:2848-2855.
Beare, P A, R J For, L W Martin, and I L Lamont (2003) Siderophore-mediated cell signalling in Pseudomonas aeruginosa: divergent pathways regulate virulence factor production and siderophore receptor synthesis. Mol Microbiol 47:195-207.
Cunliffe, H E, T R Merriman, and I L Lamont (1995) Cloning and
characterization of pvdS, a gene required for pyoverdine synthesis in Pseudomonas aeruginosa: PvdS is probably an alternative sigma factor. J Bacteriol 177:2744-2750.
Handfield, M, D E Lehoux, F Sanschagrin, M J Mahan, D E Woods, and R C Levesque (2000) In vivo-induced genes in Pseudomonas aeruginosa. Infect Immun 68:2359-2362.
McMorran, B J, H M Kumara, K Sullivan, and I L Lamont (2001) Involvement of a transformylase enzyme in siderophore synthesis in Pseudomonas aeruginosa. Microbiology 147:1517-1524.
McMorran, B J, M E Merriman, I T Rombel, and I L Lamont (1996) Characterisation of the pvdE gene which is required for pyoverdine synthesis in Pseudomonas aeruginosa. Gene 176:55-59.
Miyazaki, H, H Kato, T Nakazawa, and M Tsuda (1995) A positive regulatory gene, pvdS, for expression of pyoverdin biosynthetic genes in Pseudomonas aeruginosa PAO. Mol Gen Genet 248:17-24.
Visca, P, A Ciervo, and N Orsi (1994) Cloning and nucleotide sequence of the pvdA gene encoding the pyoverdin biosynthetic enzyme l-ornithine N5-oxygenase in Pseudomonas aeruginosa. J Bacteriol 176:1128-1140.
J-M M Meyer (2000) Pyoverdines: pigments, siderophores and potential taxonomic markers of fluorescent Pseudomonas species. Archives of Microbiology 174(3):135-42.
J Ravel and P Cornelis (2003) Genomics of pyoverdine-mediated iron uptake in pseu-domonads. Trends in Microbiology 11(5):195-200.
C Georges and J M Meyer (1995) High-molecular-mass, iron-repressed cytoplasmic proteins in fluorescent Pseudomonas: potential peptide-synthetases for pyoverdine biosynthesis. FEMS Microbiology Letters 132(1-2):9-15.
D E Lehoux, F Sanschagrin, and R C Levesque (2000) Genomics of the 35-kb pvd locus and analysis of novel pvdIJK genes implicated in pyoverdine biosynthesis in Pseudomonas aeruginosa. FEMS Microbiology Letters 190(1):141-6.
D Mossialos, U Ochsner, C Baysse, P Chablain, J-P Pirnay, N Koedam, H Budzikiewicz, D U Fernandez, M Schafer, J Ravel, and P Cornelis (2002) Identification of new, conserved, non-ribosomal peptide synthetases from fluorescent pseudomonads involved in the biosynthesis of the siderophore pyoverdine. Molecular Microbiology 45(6):1673-85.
I L Lamont and L W Martin (2003) Identification and characterization of novel pyoverdine synthesis genes in Pseudomonas aeruginosa. Microbiology (Reading, England) 149(Pt 4): 833-42.
Mootz H D, Marahiel M A (1997) The tyrocidine biosynthesis operon of Bacillus brevis: complete nucleotide sequence and biochemical characterization of functional internal adenylation domains. J Bacteriol 179 (21): 6843-50.
Trauger J W, Kohli R M, Mootz H D, Marahiel M A, Walsh C T (2000) Peptide cyclization catalysed by the thioesterase domain of tyrocidine synthetase. Nature 407 (6801): 215-8.
Pfeifer B A, Admiraal S J, Gramajo H, Cane D E, Khosla C (2001) Biosynthesis of complex polyketides in a metabolically engineered strain of E. coli. Science 291(5509): 1790-1792.
L Kaysser (2019) Built to bind: biosynthetic strategies for the formation of small-molecule protease inhibitors. Natural Product Reports DOI: 10.1039/c8np00095f
Y Ogasawara and T Dairi (2018) Peptide epimerization machineries found in microorganisms. Front Microbiol doi.org/10.3389/fmicb.2018.00156
Süssmuth R D, Mainz A (2017) Nonribosomal peptide synthesis-principles and prospects. Angew Chem Int Ed 56:3770-3821.
Challis G, Ravel J, Townsend C (2000) Predictive, structure-based model of amino acid recognition by nonribosomal peptide synthetase adenylation domains. Cell Chem Biol 7(3):211-224.
Stachelhaus T, Mootz H D, Marahiel M A (1999) The specificity-conferring code of adenylation domains in nonribosomal peptide synthetases. Chem Biol 6(8):493-505.
Bachmann B, Ravel J. (2009) In silico prediction of microbial secondary metabolic pathways from DNA sequence data. Methods in Enzymology. 458:181-217.
Khayatt B I, Overmars L, Siezen R J, Francke C (2013) Classification of the adenylation and acyl-transferase activity of NRPS and PKS systems using ensembles of substrate specific hidden Markov models. PLOS ONE 8(4) e62136 1-10.

This application contains a sequence listing which has been submitted electronically in ASCII format and is hereby incorporated by reference in its entirety. Said ASCII copy, created on 8 Sep. 2020, is named 1240_WO1_SL.txt and is 1,485,098 bytes in size.

SEQ ID NO: 1-242 are set out in the sequence listing. The database sequence information (including sequences and accession numbers) provided with this description corresponds to the information accessed online as of 11 Sep. 2020.

Any references cited in this specification are hereby incorporated by reference. All amino acid and nucleotide sequences in the references cited in this specification are hereby incorporated into this disclosure. No admission is made that any reference constitutes prior art. Nor does discussion of any reference constitute an admission that such reference forms part of the common general knowledge in the art, in Australia or in any other country.

Persons of ordinary skill can utilise the disclosures and teachings herein to produce other embodiments and variations without undue experimentation. All such embodiments and variations are considered to be part of this invention.

Accordingly, one of ordinary skill in the art will readily appreciate from the disclosure that later modifications, substitutions, and/or variations performing substantially the same function or achieving substantially the same result as embodiments described herein may be utilised according to such related embodiments of the present invention. Thus, the invention is intended to encompass, within its scope, the modifications, substitutions, and variations to processes, manufactures, compositions of matter, compounds, means, methods, and/or steps disclosed herein.

The description herein may contain subject matter that falls outside of the scope of the claimed invention. This subject matter is included to aid understanding of the invention.

1. A non-naturally occurring non-ribosomal peptide synthetase (NRPS) module, which comprises, in an N-terminal to C-terminal direction: (1) an amino acid sequence from a first NRPS module comprising a C domain from the C1 motif to the C7 motif, joined to (2) an amino acid sequence from a second NRPS module comprising an A domain or a fragment thereof.

2. The NRPS module of claim 1, wherein the amino acid sequence from the second NRPS module begins at a site 1 to 24 amino acids, or 1 to 14 amino acids, following the terminal helix of the C domain of the first NRPS module.

3. The NRPS module of claim 1 or claim 2, wherein the amino acid sequence from the second NRPS module comprises an A domain of the second NRPS module, the A domain encompassing the linker helix to the A10 motif.

4. The NRPS module of claim 1 or claim 2, wherein the amino acid sequence from the second NRPS module begins at a site within the terminal helix of the C domain of the first NRPS module.

5. The NRPS module of claim 1 or claim 2, wherein the amino acid sequence from the second NRPS module begins at a site within the linker helix of the A domain of the first NRPS module.

6. The NRPS module of claim 1 or claim 2, wherein the amino acid sequence from the second NRPS module begins at a site immediately following the C-terminus of the terminal helix of the C domain of the first NRPS module.

7. The NRPS module of claim 1 or claim 2, wherein the amino acid sequence from the second NRPS module begins at a site immediately preceding the N-terminus of the linker helix of the A domain of the first NRPS module.

8. The NRPS module of claim 1 or claim 2, wherein the amino acid sequence from the second NRPS module begins at a site immediately preceding the C-terminus of the linker helix of the A domain of the first NRPS module.

9. The NRPS module of any one of claims 1 to 8, wherein the amino acid sequence from the second NRPS module ends at a site preceding the first helix of the T domain of the first NRPS module.

10. The NRPS module of any one of claims 1 to 8, wherein the amino acid sequence from the second NRPS module ends at a site in the first NRPS module encompassing: the residue immediately following the A domain binding pocket to 20 residues following the A10 motif.

11. The NRPS module of any one of claims 1 to 8, wherein the amino acid sequence from the second NRPS module ends at a site in the first NRPS module encompassing: the residue immediately following the A domain binding pocket to 10 residues following the A10 motif.

12. The NRPS module of any one of claims 1 to 11, wherein the first NRPS module and the second NRPS module have different substrate specificity.

13. The NRPS module of any one of claims 1 to 12, wherein the A domain of the first NRPS module and the A domain of the second NRPS module share less than 40%, less than 50%, less than 60% or less than 70% amino acid sequence identity.

14. The NRPS module of any one of claims 1 to 13, wherein the C domain of the first NRPS module and the C domain of the second NRPS module share less than 40%, less than 50%, less than 60% or less than 70% amino acid sequence identity.

15. The NRPS module of any one of claims 1 to 14, wherein the region intervening the A domain and the C domain of the first NRPS module and the region intervening the A domain and the C domain of the second NRPS module share less than 40%, less than 50%, less than 60% or less than 70% amino acid sequence identity.

16. The NRPS module of any one of claims 1 to 15, wherein the A domain binding pocket of the second NRPS module differs from the A domain binding pocket of the first NRPS module by 1 or more amino acids.

17. The NRPS module of any one of claims 1 to 15, wherein the A domain binding pocket of the second NRPS module differs from the A domain binding pocket of the first module by 2 or more, 3 or more, 4 or more, 5 or more, 6 or more, 7 or more, or 8 amino acids.

18. The NRPS module of claim 16 or claim 17, wherein one or more different amino acids in the A domain of the second NRPS module are selected from one or more of eight amino acids that determine specificity of the A domain.

19. The NRPS module of any one of claims 1 to 18, wherein downstream to the amino acid sequence from the second NRPS module includes a C-terminal sequence from the first NRPS module.

20. The NRPS module of claim 19, wherein the C-terminal sequence comprises a domain from the first NRPS module.

21. The NRPS module of any one of claims 1 to 18, wherein downstream to the amino acid sequence from the second NRPS module includes a C-terminal sequence from a third NRPS module.

22. The NRPS module of claim 21, wherein the C-terminal sequence comprises a domain from the third NRPS module.

23. The NRPS module of any one of claims 1 to 22, wherein the non-naturally occurring non-ribosomal peptide synthetase module has enzymatic activity.

24. An NRPS enzyme comprising the NRPS module of any one of claims 1 to 23.

25. A polynucleotide encoding the NRPS module of any one of claims 1 to 23.

26. A nucleic acid construct encoding the NRPS module of any one of claims 1 to 23.

27. A nucleic acid library, wherein a nucleic acid in the library encodes an NRPS module of any one of claims 1 to 23.

28. A host cell comprising the polynucleotide of claim 25 or the nucleic acid construct of claim 26.

29. A host cell comprising the nucleic acid library of claim 27.

30. A method for generating the NRPS module of any one of claims 1 to 23.

31. A method for production of a non-ribosomal peptide, the method comprising culturing the host cell of claim 28 or claim 29 to produce the non-ribosomal peptide.

32. A method for the production of a non-ribosomal peptide, the method comprising use of the NRPS module of any one of claims 1 to 23, the nucleic acid construct of claim 26, the library of claim 27, or the host cell of claim 28 or claim 29.

33. A kit comprising the NRPS module of any one of claims 1 to 23, the nucleic acid construct of claim 26, the library of claim 27, or the host cell of claim 28 or claim 29.
